(19) United States
(12) Patent Application Publication
(10) Pub. No.: US 2024/0263164 A1
(43) Pub. Date: Aug. 8, 2024

(54) Nuclease-Based RNA Depletion

(71) Applicant: Illumina, Inc., San Diego, CA (US)

(72) Inventors: Scott Kuersten, Madison, WI (US); Frederick W. Hyde, Monona, WI (US); Asako Tetsubayashi, Madison, WI (US)

(73) Assignee: Illumina, Inc., San Diego, CA (US)

(21) Appl. No.: 18/610,023

(22) Filed: Mar. 19, 2024

Related U.S. Application Data
(63) Continuation of application No. 17/812,683, filed on Jul. 14, 2022, now Pat. No. 11,976,271.
Application No. 17/812,683 is a continuation of application No. 16/721,468, filed on Dec. 19, 2019, now Pat. No. 11,421,216.
(60) Provisional application No. 62/847,797, filed on May 14, 2019.
(60) Provisional application No. 62/783,869, filed on Dec. 21, 2018.

(51) Int. Cl. C12N 15/10; C12Q 1/6806; C12Q 1/6848; C12Q 1/6876

(52) U.S. Cl. CPC C12N 15/1003; C12N 15/1024; C12Q 1/6806; C12Q 1/6848; C12Q 1/6876

(57) ABSTRACT

The present disclosure is related to methods and materials for depleting unwanted RNA species from a nucleic acid sample. In particular, the present disclosure describes how to remove unwanted rRNA, tRNA, mRNA or other RNA species that could interfere with the analysis, manipulation and study of target RNA molecules in a sample.

### CROSS-REFERENCE TO RELATED APPLICATIONS

This application is a continuation of U.S. patent application Ser. No. 17/812,683, filed Jul. 14, 2022, which is a continuation of U.S. patent application Ser. No. 16/721,468, filed Dec. 19, 2019, which claims the benefit of priority of US Provisional Application Nos. 62/783,869, filed Dec. 21, 2018, and 62/847,797, filed May 14, 2019, each of which is incorporated by reference herein in its entirety for any purpose.

### SEQUENCE LISTING

The present application is filed with a Sequence Listing in electronic format. The Sequence Listing is provided as a file entitled “2024-03-19_01243-0012-02US_Seq_List_ST26” created on Mar. 19, 2024, which is 534,283 bytes in size. The information in the electronic format of the sequence listing is incorporated herein by reference in its entirety.

### BACKGROUND

Unwanted RNA in a nucleic acid sample, like a nucleic acid sample taken from human cells or tissues, can complicate the analysis of that sample, analysis such as gene expression analysis, microarray analysis and sequencing of a sample. As ribosomal RNA (rRNA) comprises roughly 95% of the RNA in a cell its presence is one example of an RNA species that can interfere and obfuscate results of a target nucleic acid in a sample, or those nucleic acids that a researcher or diagnostician might want to understand more about. For example, unwanted rRNA species can make it especially difficult to analyze RNA molecules of interest in a sample, such as tRNA or mRNA. This is an ever-present problem particularly for tissues that have been fixed, for example fixed by formalin and then embedded in wax such as formalin fixed paraffin embedded (FFPE) tissues from biopsies. Without removing the rRNA species from FFPE tissues they can interfere with the measurement and characterization of target RNA in the tissue thereby making it extremely difficult to derive medically actionable information from the target RNAs such as disease and cancer identification, potential treatment options and disease or cancer diagnosis and prognosis. While FFPE tissue is an example, the same issues with rRNA hold true for samples of all kinds such a blood, cells, and other types of nucleic acid containing samples.

Current commercially available methods for depleting undesired RNA from a nucleic sample include RiboZero® (Epicentre) and NEBNext® rRNA Depletion kits (NEB) and RNA depletion methods as described in U.S. Pat. Nos. 9,745,570 and 9,005,891. However, these methods, while being useful in depleting RNA, have their own disadvantages, including ease of use, high sample input requirements, technician hands on time, cost, and/or efficiency in depleting undesired RNA from a sample. What are needed are materials and methods that can more easily or cost effectively deplete unwanted RNA species from a sample thereby unlocking information in the target RNA which might have been hidden such as rare or difficult to identify sequence variants. Straightforward and reliable methods as described in this disclosure can greatly increase the availability of target RNA molecules for testing purposes, thereby discovering the information they hold about the sample and the organism from which it derives.

### SUMMARY OF THE INVENTION

Nucleic acid samples such as those from eukaryotes or prokaryotes contain multitude nucleic acids, many of which are not of interest to a researcher. Researchers oftentimes wish to study a specific type of a nucleic acid, such as either DNA or RNA. When studying RNA, the sample of interest can contain many different types of RNA species that can overwhelm and hide the target RNA that is the focus of study. As such, RNA depletion refers to removing unwanted RNA and/or DNA species from a nucleic acid sample thereby leaving a nucleic acid sample enriched with the desired RNA for study.

The present disclosure provides a solution for depleting a nucleic acid sample of an overabundance of unwanted RNA species prior to further study. For example, an RNA sample of interest not only includes the target RNA to be studied, but also includes abundant transcripts like rRNA, globin mRNA, viral contaminates, or any other unwanted nucleic acids that can dominate the sample and swamp out the target of interest, thereby greatly decreasing a researcher's ability to accurately analyze the desired portion of the transcriptome.

Therefore, depleting unwanted RNA from a nucleic acid sample prior to analysis, such as expression microarrays or sequencing, increases the specificity and accuracy of analysis for the desired RNA targets. In the present disclosure, depletion of off-target RNA through degradation of specific DNA:RNA hybrids allows for efficient removal of unwanted RNA species from a sample prior to library preparation and analysis. Once a sample is depleted of unwanted RNA species, the remaining target RNA can be converted to cDNA. Obtaining actionable data as a result of a robust sample can lead to a better understanding and potential treatment options for cancer prognostics and diagnostics, a better understanding of our microbiome and its importance in our and other eukaryotic systems, a more thorough understanding of expression analysis of genes of interest, and the like.

In one embodiment, the present disclosure describes a method for depleting off-target RNA molecules from a nucleic acid sample comprising: a) contacting a nucleic acid sample comprising at least one RNA or DNA target sequence and at least one off-target RNA molecule with a probe set comprising at least two DNA probes complementary to discontiguous sequences along the full length of the at least one off-target RNA molecule, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid; and b) contacting the DNA:RNA hybrids with a ribonuclease that degrades the RNA from the DNA:RNA hybrids, thereby degrading the off-target RNA molecules in the nucleic acid sample to form a degraded mixture.

In one embodiment, the present disclosure relates to a composition comprising a probe set comprising at least two DNA probes complementary to discontiguous sequences along the full length of at least one off-target RNA molecule (e.g., at least 5 or at least 10 bases apart along the full length) in a nucleic acid sample. In some embodiments, the composition also comprises a ribonuclease capable of degrading RNA in a DNA:RNA hybrid. In another embodiment, the present disclosure relates to a composition comprising a probe set comprising at least two DNA probes hybridized to at least one off-target RNA molecule, wherein each DNA probe is hybridized at least 5, or at least 10, bases apart along the length of the off-target RNA molecule from any other DNA probe in the probe set.

In one embodiment, the present disclosure describes a kit comprising a probe set comprising at least two DNA probes complementary to discontiguous sequences along the full length of at least one off-target rRNA molecule (e.g., at least 5 bases apart or at least 10 bases apart along the full length) in a nucleic acid sample and a ribonuclease capable of degrading RNA in a DNA:RNA hybrid.

In one embodiment, the present disclosure describes a method of supplementing a probe set for use in depleting off-target RNA nucleic acid molecules from a nucleic acid sample comprising: a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid; b) contacting the DNA:RNA hybrids with a ribonuclease that degrades the RNA from the DNA:RNA hybrids, thereby degrading the off-target RNA molecules in the nucleic acid sample to form a degraded mixture; c) separating the degraded rRNA from the degraded mixture; d) sequencing the remaining RNA from the sample; e) evaluating the remaining RNA sequences for the presence of off-target RNA molecules from the first species, thereby determining gap sequence regions; and f) supplementing the probe set with additional DNA probes complementary to discontiguous sequences in one or more of the gap sequence regions.

### BRIEF DESCRIPTION OF THE FIGURES

FIG. 1 shows an exemplary workflow for performing depletion of RNA species from a sample. Step 1 includes nucleic acid denaturation followed by addition of depletion DNA probes and hybridization of the probes with the unwanted RNA species, thereby creating DNA:RNA hybrids. Step 2 includes digestion of the RNA from the DNA:RNA hybrids using a ribonuclease such as RNase H. Step 3 includes digesting residual DNA probes from the degraded mixture by addition of DNase. Step 4 includes capturing the remaining target RNA in the sample, which is optionally followed by additional manipulations that will eventually result in a sample depleted of unwanted RNA species that can be sequenced, exposed to microarray expression analysis, qPCR, or other analysis techniques.

FIGS. 2A-2C show exemplary data for rRNA depletion from a sample of B. subtilis when formamide is added to the rRNA depletion workflow (2A) 0% formamide, (2B) 25% formamide, (2C) 45% formamide). In each panel, the X axis lists the detected rRNA species and the Y axis shows percent depletion through percent sequence reads.

FIG. 3 shows exemplary next-generation sequencing (NGS) sequence data for rRNA depleted samples of Human Brain RNA (HBR) and Universal Human RNA (UHR) comparing different amounts of sequenced sample (100 ng, 10 ng, or 1 ng).

FIG. 4 shows exemplary NGS sequence data for rRNA depleted samples from mouse RNA and rat RNA using different concentrations of formamide (0%, 25%, 45%) added to the rRNA depletion workflow.

FIG. 5 shows exemplary data from removal of rRNA from different microbial species using low sample inputs comparing RiboZero® and RNase H enzymatic removal rRNA depletion methodologies. All sample read-depths were normalized. The X axis shows the rRNA depletion method (RZ=RiboZero or ED=RNase H enzymatic depletion method) and the Y axis shows the % rRNA reads.

FIG. 6 shows exemplary transcript detection data at various read depths for B. subtilis and E. coli following RNase H rRNA depletion (ED) on the left side of the graph compared to no rRNA depletion (None) on the right side of the graph. The X axis shows the sequencing reads (M) and the Y axis shows the number of transcripts detected.

FIGS. 7A-7B show exemplary graphs for gene expression pairwise linear regression data demonstrating the reproducibility of the disclosed methods for rRNA depletion. Panel 7A exemplifies two E. coli replicate gene expression levels and Panel 7B exemplifies two B. subtilis replicate gene expression levels. Both bacterial types demonstrate high correlation between gene expression level replicates following RNase H rRNA depletion.

FIG. 8 shows exemplary triplicate rRNA read data for a 20 strain (MSA-2002, left side) and a 12 strain (MSA-2006, right side) mixed sample. The mixed sample triplicates were rRNA depleted by the RiboZero method (RZ) or the RNase H (ED) depletion method described herein. RNA input for the MSA2002 samples was 10 ng while that for the MSA2006 was 80 ng. The X axis shows the rRNA depletion method and the Y axis shows the % rRNA reads.

FIG. 9 shows sequencing read coverage of the mouse mitochondrial 12S (mt-Rnr1 and 16S (mt-Rnr2) rRNA loci (bottom of the figure) and the effect of the 333 DNA probe set (SEQ ID NOs: 1-333) on depleting mouse 16S rRNA from universal mouse reference RNA (UMR) samples. Squares indicate location of 90% match over 50 base length or 70% match over 30 base pair length with the 333 DNA probe set. In the absence of additional mouse and rat probes, gaps without probe coverage correspond to peaks in residual or undepleted rRNA for the two replicates (Rep 1 and Rep2) shown at the top of the figure.

### DETAILED DESCRIPTION

Creating nucleic acid libraries from RNA for sequencing is oftentimes difficult due to an abundance of unwanted transcripts such as ribosomal RNA, globin mRNA, viral contaminants, and the like that can dominate a sample and swamp out the RNA sequences of interest. If the unwanted transcripts are not removed, analysis of the transcriptome which would have prognostic, diagnostic or research benefit could be compromised. Therefore, depleting unwanted RNA from a nucleic acid sample prior to analysis such as sequencing or other downstream applications can increase the specificity and accuracy of the desired analysis.

The present disclosure describes methods and materials useful in depleting unwanted RNA species from a nucleic acid sample such that the RNA of importance can be studied and is not lost in the sea of undesired RNA transcripts.

Compared to existing methods for RNA depletion, the disclosed method can utilize smaller amounts of input total RNA while still maintaining comparable performance metrics. Therefore, the disclosed method can be used when a researcher has small amounts of starting material which other methods would not be able to accommodate. Further, the disclosed method can be performed with one pool of probes that target a variety of different organismal unwanted RNA species simultaneously without compromising depletion efficiency. For example, the present disclosure can simultaneously deplete unwanted eukaryotic and prokaryotic RNA species from an RNA sample, including but not limited to human, bacterial, viral and/or Archaea sources of unwanted RNA.

A nucleic acid sample or mixture refers to a sample that contains RNA or DNA or both, including both undesired (off-target or unwanted) and desired (target) nucleic acids. The DNA or RNA in the sample can be either unmodified or modified and includes, but is not limited to, single or double stranded DNA or RNA or derivatives thereof (e.g., some regions of the DNA or RNA are double stranded whereas concurrently other regions of the DNA or RNA are single stranded) and the like. In general, a nucleic acid sample includes all chemically, enzymatically, and/or metabolically modified forms of nucleic acids as well as all unmodified forms of nucleic acids, or combinations thereof. A nucleic acid sample can contain both wanted and unwanted nucleic acids such as genomic DNA or total cellular RNA or a combination of both. Unwanted nucleic acids include those nucleic acids from eukaryotes that are not targeted for study as well as contaminating nucleic acids from bacteria, viruses, Archaea species, and the like. Wanted or desired nucleic acids are those nucleic acids that are the basis or focus of study, the target nucleic acids. For example, a researcher may desire to study mRNA expression analysis, wherein rRNA, tRNA and DNA would be considered unwanted nucleic acids and mRNA is the target nucleic acid. As well, study of total RNA could be desired, whereas the rRNA, mRNA and DNA would be considered unwanted or undesired nucleic acids and the total RNA the target nucleic acid. Unwanted RNA includes, but is not limited to, ribosomal RNA (rRNA), mitochondrial rRNA, nuclear rRNA, mRNA such as globin RNAs, or transfer or tRNA, or a mixture thereof. In some embodiments, off-target RNA is rRNA. In some embodiments, off-target RNA is globin mRNA.

For example, a nucleic acid sample could contain the desired messenger RNA (mRNA) or total RNA while also including undesired ribosomal RNA (rRNA), transfer RNAs (tRNA) and perhaps undesired DNA. General methods for RNA extraction from a gross sample, like blood, tissue, cells, fixed tissues, etc., are well known in the art, as found in Current Protocols for Molecular Biology (John Wiley & Sons) and multitude molecular biology methods manuals. RNA isolation can be performed by commercially available purification kits, for example Qiagen RNeasy mini-columns, MasterPure Complete DNA and RNA Purification Kits (Epicentre), Parrafin Block RNA Isolation Kit (Ambion), RNA-Stat-60 (Tel-Test) or cesium chloride density gradient centrifugation. The current methods are not limited by how the RNA is isolated from a sample prior to RNA depletion.

There is an inherent skepticism that mixing probes targeting bacterial rRNA and human rRNA into the same pool would lead to extensive off-target depletion of desirable transcripts (Mauro et al., Proc. Natl. Acad. Sci. USA 1997, 94:422-427; Mignone and Pesole, Appl. Bioinformatics 2002, 1:145-54). Surprisingly, research performed while developing the disclosed methods demonstrates this isn't the case, as the specificity of the DNA probe hybridization with the unwanted RNA transcripts results in a sample efficiently depleted of unwanted RNA species. It was also discovered that the addition of a destabilizer such as formamide helps remove some unwanted RNA that was shown to be more problematic to deplete if formamide was not present. Although it is not necessary to understand the way in which formamide helps in removing those RNA, it is thought that the formamide may serve to relax structural barriers in the unwanted RNA so that the DNA probes can bind more efficiently. Further, the addition of formamide has demonstrated the added benefit of improving the detection of some non-targeted transcripts possibly by denaturing/relaxing regions of the mRNAs, for example, that have very stable secondary or tertiary structures and are not normally well represented well in other library preparation methods.

### Nucleic Acid Samples or Mixtures

The present disclosure is not limited to the source of a nucleic acid sample, for example, the source could be from eukaryotes or prokaryotes including but not limited to humans, non-human primates, mammals, birds, reptiles, plants, bacteria, viruses, nucleic acids found in soils, water or other liquids and other environmental samples. The sample could be obtained from cells, tissues, organs, the environment, lysates, etc. and could come from any state of a sample such as fresh, frozen, lyophilized and reconstituted, or a fixed sample such as from a tissue or biopsy specimen that has been formalin fixed paraffin embedded (FFPE) or other cytological or histological sample manipulation.

The nucleic acid sample that could benefit from the RNA depletion methods could be from any species, eukaryotic or prokaryotic, such as humans, non-humans, mice, rats, bacteria, etc. and could include single or multiple species in one sample. Additionally, the present depletion methods could be used on fresh or preserved samples such as biopsy or tissue samples, including samples that have been processed using formalin and embedded in paraffin (e.g., formalin fixed paraffin embedded, FFPE, samples). In some embodiments, a nucleic acid sample is from a human or non-human source such as non-human eukaryotes, bacteria, viruses, plants, soil or a mixture thereof. Once a sample is depleted of unwanted RNA species, the remaining desired targets can be converted to cDNA for further processing as known to those skilled in the art.

In some embodiments, a nucleic acid sample is from a human or a non-human primate. In some embodiments, a nucleic acid sample is from a rat or a mouse. In some embodiments, a nucleic acid sample comprises nucleic acids of non-human origin. In some embodiments, nucleic acids of non-human origin are from non-human eukaryotes, bacteria, viruses, plants, soil, or a mixture thereof.

### Depletion Methods

As such, unwanted or undesired RNA in a nucleic acid sample is depleted by the described methods. The unwanted RNA is converted to a DNA:RNA hybrid by hybridizing partially or completely complementary DNA probes to the unwanted RNA molecules. Methods for hybridizing nucleic acid probes to nucleic acids are well established in the sciences and whether a probe is partially or completely complementary with the partner sequence, the fact that a DNA probe hybridizes to the unwanted RNA species following washes and other manipulations of the sample demonstrates a DNA probe that can be used in methods of the present disclosure. The unwanted RNA set for depletion can be from any eukaryotic species, for example, human, mice, rats, etc., where depletion of RNA from a sample might result in more favorable downstream studies such as sequencing (e.g., fewer results from unwanted nucleic acid species). DNA can also be considered an unwanted nucleic acid if the target for study is an RNA, at which point DNA can also be removed by depletion.

In one embodiment, the present disclosure describes a method for depleting off-target RNA molecules from a nucleic acid sample comprising:
a) contacting a nucleic acid sample comprising at least one RNA or DNA target sequence and at least one off-target RNA molecule with a probe set comprising at least two DNA probes complementary to discontiguous sequences along the full length of the at least one off-target RNA molecule, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid; and
b) contacting the DNA:RNA hybrids with a ribonuclease that degrades the RNA from the DNA:RNA hybrids, thereby degrading the off-target RNA molecules in the nucleic acid sample to form a degraded mixture.

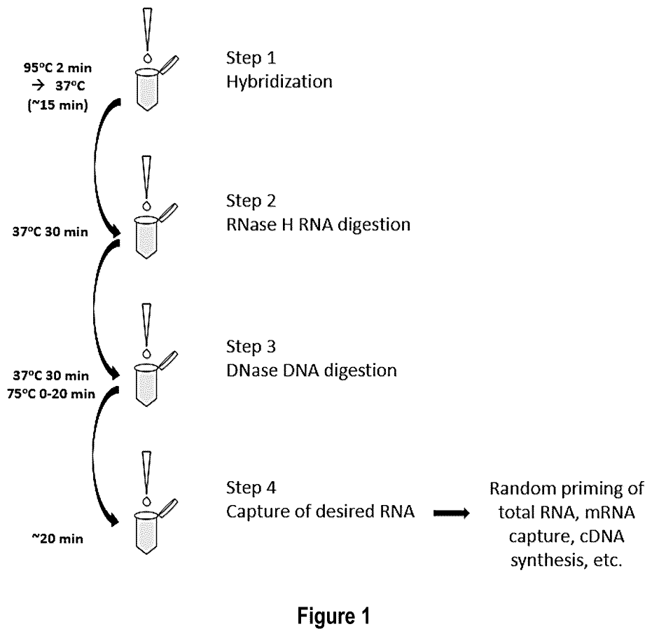

In one embodiment, an RNA sample is denatured in the presence of the DNA probes. An exemplary workflow is demonstrated in FIG. 1. In the example in FIG. 1, the DNA probes are added to the denatured RNA sample (denatured at 95° C. for 2 min.) whereupon cooling the reaction to 37° C. for 15-30 min results in hybridization of the DNA probes to their respective target RNA sequences thereby creating DNA:RNA hybrid molecules.

In some embodiments, contacting with the probe set comprises treating the nucleic acid sample with a destabilizer. In some embodiments, a destabilizer is heat or a nucleic acid destabilizing chemical. In some embodiments, a nucleic acid destabilizing chemical is betaine, DMSO, formamide, glycerol, or a derivative thereof, or a mixture thereof. In some embodiments, a nucleic acid destabilizing chemical is formamide or a derivative thereof, optionally wherein the formamide or derivative thereof is present at a concentration of from about 10 to 45% of the total hybridization reaction volume. In some embodiments, treating the sample with heat comprises applying heat above the melting temperature of the at least one DNA:RNA hybrid.

In some embodiments, formamide is added to the hybridization reaction regardless of RNA sample source (e.g., human, mouse, rat, etc.). For example, in some embodiments, hybridizing to the DNA probes is performed in the presence of at least 3%, 5%, 10%, 20%, 25%, 30%, 35%, 40%, or 45% by volume of formamide. In one embodiment, a hybridization reaction for RNA depletion includes approximately 25% to 45% by volume of formamide.

Following the hybridization reaction, a ribonuclease that degrades RNA from a DNA:RNA hybrid is added to the reaction. In some embodiments, a ribonuclease is RNase H or Hybridase. RNase H (NEB) or Hybridase (Lucigen) are examples of enzymes that will degrade RNA from a DNA:RNA hybrid. Degradation by a ribonuclease such as RNase H or Hybridase degrades the RNA into small molecules that can then be removed. For example, RNase H is reported to digest RNA from a DNA:RNA hybrid approximately every 7-21 bases (Schultz et al., J. Biol. Chem. 2006, 281:1943-1955; Champoux and Schultz, FEBS J. 2009, 276:1506-1516). In some embodiments, the digestion of the RNA of the DNA:RNA hybrid can occur at 37° C. for approximately 30 min as described in FIG. 1, Step 2, and Example 1.

In some embodiments, following DNA:RNA hybrid molecule digestion, the remaining DNA probes and any off-target DNA in the nucleic acid sample are degraded. Thus, in some embodiments, the methods comprise contacting the ribonuclease-degraded mixture with a DNA digesting enzyme, thereby degrading DNA in the mixture. In some embodiments, the digested sample is exposed to a DNA digesting enzyme such as DNase I, which degrades the DNA probes. The DNase DNA digestion reaction is incubated, for example, at 37° C. for 30 min, after which point the DNase enzyme can be denatured at 75° C. for a period of time as necessary to denature the DNase, for example for up to 20 min.

In some embodiments, the depletion method comprises separating the degraded RNA from the degraded mixture. In some embodiments, separating comprises purifying the target RNA from the degraded RNA (and degraded DNA if present), for example, using a nucleic acid purification medium, such as RNA capture beads, such as RNAClean XP beads (Beckman Coulter). Thus, in some embodiments, following the enzymatic digestion(s), the target RNA can be enriched by removing the degraded products while leaving the desired and longer RNA targets behind. Suitable enrichment methods include treating the degraded mixture with magnetic beads which bind to the desired fragment size of the enriched RNA targets, spin columns, and the like. In some embodiments, magnetic beads such as AMPure XP beads, SPRISelect beads, RNAClean XP beads (Beckman Coulter) can be used, as long as the beads are free of RNases (e.g., Quality Controlled to be RNase free). These beads provide different size selection options for nucleic acid binding, for example RNAClean XP beads target 100 nt or longer nucleic acid fragments and SPRISelect beads target 150 to 800 nt nucleic acid fragments and do not target shorter nucleic acid sequences such as the degraded RNA and DNA that results from the enzymatic digestions of RNase H and DNase. If mRNA is the target RNA to be studied, then the mRNA can be further enriched by capture using, for example, beads that comprise oligodT sequences for capturing the mRNA adenylated tails. Methods of mRNA capture are well known by skilled artisans.

Once the target RNA has been purified away from the reaction components including the undesired degraded nucleic acids, additional sample manipulation can occur. In the present disclosure, Examples 2 and 3 provide exemplary workflows for cDNA synthesis from the enriched target total RNA followed by an exemplary library preparation workflow that is typical for subsequent sequencing on, for example, an Illumina sequencer. However, it should be understood that these workflows are exemplary only and a skilled artisan will understand that the enriched RNA can be used in multitude additional applications such as PCR, qPCR, microarray analysis, and the like either directly or following additional manipulation such as converting the RNA to cDNA by using established and will understood protocols.

The methods described herein for RNA depletion will result in a sample enriched with the target RNA molecules. For example, the methods described herein result is a depleted RNA sample comprising less than 15%, 13%, 11%, 9%, 7%, 5%, 3%, 2% or 1% or any range in between of the unwanted RNA species. The enriched RNA sample then comprises at least 99%, 98%, 97%, 95%, 93%, 91%, 89% or 87% or any range in between of the target total RNA. Once the sample has been enriched it can be used for library preparation or other downstream manipulations.

### DNA Probe Sets DNA Probes

A DNA probe refers to a single stranded DNA oligonucleotide that has sequence complementarity to unwanted RNA species. The DNA probe sequence can be partly or completely complementary to the undesired RNA for depletion in the nucleic acid sample. The unwanted RNA for depletion includes, but is not limited to, rRNA, tRNA, and mRNA, and mixtures thereof. In some embodiments, each DNA probe is from about 10 and 100 nucleotides long, or from about 20 and 80 nucleotides long, or from about 40 to 60 nucleotides long, or about 50 nucleotides long. The DNA probes are capable of hybridizing to the unwanted RNA species, thereby creating DNA:RNA hybrid molecules. While in some embodiments, at least two DNA probes hybridize to a particular off-target RNA molecule, the DNA probes do not cover the entire length of an unwanted RNA molecule sequence. For example, in some embodiments, a probe set leaves gaps or regions of the unwanted RNA without a complementary DNA probe in the probe set. The DNA probes hybridize, completely or partly, to the unwanted RNA in a non-overlapping manner, leaving gaps of at least 5, 10, 15, 20, 30, 40, 50, 60, 70, 80 or more nucleotides between the resultant DNA:RNA hybrids. Thus, in some embodiments, each DNA probe is hybridized at least 5, or at least 10, bases apart along the full length of the at least one off-target RNA molecule from any other DNA probe in the probe set. As such, the unwanted RNA in its entirety is not completely hybridized to DNA probes. Further, the present disclosure provides for a plurality of DNA probes that hybridize to a single RNA for depletion as such there is not a “one DNA probe for one RNA”, but instead multiple discontinuous DNA probes in a probe set that target a given unwanted RNA. For example, in some embodiments, for a given RNA set for depletion, a DNA probe set is used where each probe is approximately 20-80 nucleotides long and each probe hybridizes to the unwanted RNA anywhere from 5-15 nucleotides away from another DNA probe in the set. A DNA probe can be completely or partially complementary to a particular location on the RNA to be depleted, for example the DNA probe sequence can be at least 80%, 85%, 90%, 95%, or 100% complementary, or any range in between, to the target location on an RNA transcript to be depleted. The only limitation to complementarity is that the DNA probe should hybridize to the target RNA to be depleted in such a manner that a DNA:RNA hybrid results that is enzymatically digestible as described herein. In some cases, mRNA is the target of interest and not targeted for depletion, in which case the DNA probes do not comprise a polyT sequence so that the probes will not hybridize to mRNA species. In some embodiments, the DNA probes do not comprise a tag with a capture moiety such as biotin, avidin, streptavidin, or a magnetic bead that would allow for depletion of the hybrid by physical means, whereas in other embodiments the DNA probes do comprise a tag with a capture moiety such as biotin, avidin, streptavidin, or a magnetic bead that would allow for depletion of the hybrid by physical means.

In some embodiments, a probe set comprises at least DNA probes that hybridize to off-target RNA molecules from humans and bacteria. In some embodiments, a probe set comprises at least DNA probes that hybridize to off-target RNA molecules from humans, bacteria, and Archaea. In some embodiments, a probe set comprises at least DNA probes that hybridize to off-target RNA molecules from humans, bacteria, mouse, and rat. In some embodiments, a probe set comprises at least DNA probes that hybridize to off-target RNA molecules from humans, bacteria, mouse, rat, and Archaea. In some embodiments, the off-target RNA molecules from bacteria are from Gram-positive bacteria or Gram-negative bacteria, or a mixture thereof. In some embodiments, a probe set comprises at least two DNA probes that hybridize to one or more off-target RNA molecules from an Archaea species. In some embodiments, a probe set comprises at least two DNA probes complementary to two or more rRNA sequences from an Archaea species.

In some embodiments, a probe set comprises at least two DNA probes that hybridize to at least one, or at least two, off-target RNA molecules selected from 28S, 23S, 18S, 5.8S, 5S, 16S, 12S, HBA-A1, HBA-A2, HBB, HBB-B1, HBB-B2, HBG1, and HBG2. In some embodiments, the probe set comprises at least two DNA probes complementary to two or more rRNA sequences selected from the group consisting of 28S, 23S, 18S, 5.8S, 5S, 16S, 12S, HBA-A1, HBA-A2, HBB, HBB-B1, HBB-B2, HBG1, and HBG2. In some embodiments, a probe set comprises at least two DNA probes that hybridize to one or more, or two or more, off-target RNA molecules selected from 28S, 18S, 5.8S, 5S, 16S, and 12S from humans. In some embodiments, a probe set comprises at least two DNA probes that hybridize to one or more, or two or more, off-target RNA molecules from rat and/or mouse, optionally selected from rat 16S, rat 28S, mouse 16S, and mouse 28S, and combinations thereof. In some embodiments, a probe set comprises at least two DNA probes that hybridize to one or more off-target RNA molecules selected from HBA-A1, HBA-A2, HBB, HBB-B1, HBB-B2, HBG1, and HBG2 from hemoglobin. In some embodiments, a probe set comprises at least two DNA probes that hybridize to one or more off-target RNA molecules selected from 23S, 16S, and 5S from Gram positive and/or Gram negative bacteria. Globin mRNAs for depletion can include, but are not limited to, those found in rodents such as mouse or rat including HBA-A1, HBA-A2, HBB, HBB-B1, HBB-B2, and those found in humans including HBA-A1, HBA-A2, HBB, HGB1 and HGB2. Mitochondrial rRNAs suitable for depletion include 18S and 12S (humans and rodents). Nuclear rRNAs suitable for depletion include 28S, 18S, 5.8S and 5S (humans and rodents) and prokaryotic rRNAs including 5S, 16S and 23S. In some samples, the depletion of rRNAs from Archaea species may also be desired, such as rRNAs 23S, 16S or 5S. In further embodiments, the probe set comprises at least two DNA probes complementary to two or more rRNA sequences selected from the group consisting of Gram positive or Gram negative bacterial rRNA 5S, 16S and 23S. In some embodiments, the probe set comprises at least two (or at least five, or at least 10, or at least 20) DNA probes complementary to each of human 28S, 18S, 5.8S, 5S, 16S, and 12S, globin mRNA HBA-A1, HBA-A2, HBB, HBG1, and HBG2, and Gram positive or Gram negative bacterial rRNA 5S, 16S and 23S. In some embodiments, the probes to a particular off-target RNA molecule are complementary to about 80 to 85% of the sequence of the off-target RNA molecule, with gaps of at least 5, or at least 10 bases between each probe hybridization site.

In some embodiments, a probe set comprises two or more, or five or more, or 10 or more, or 25 or more, or 50 or more, or 100 or more, or 150 or more, or 200 or more, or 250 or more, or 300 or more, or 333 sequences from SEQ ID NOs: 1-333 (human, Gram-positive bacteria, and Gram-negative bacteria). In some embodiments, a probe set comprises two or more, or five or more, or 10 or more, or 25 or more, or 50 or more, or 100 or more, or 150 or more, or 200 or more, or 250 or more, or 300 or more, or 350 or more, or 400 or more, or 428 sequences from SEQ ID NOs: 1-428 (human, Gram-positive bacteria, Gram-negative bacteria, Archaea, mouse, and rat). In some embodiments, a probe set comprises two or more, or five or more, or 10 or more, or 25 or more, or 50 or more, or 100 or more, or 150 or more, or 200 or more, or 250 or more, or 300 or more, or 350 or more, or 377 sequences from SEQ ID NOs: 1-377 (human, Gram-positive bacteria, Gram-negative bacteria, and Archaea). In some embodiments, a probe set comprises two or more, or five or more, or 10 or more, or 25 or more, or 50 or more, or 100 or more, or 150 or more, or 200 or more, or 250 or more, or 300 or more, or 350 or more, or 384 sequences from SEQ ID NOs: 1-333 (human, Gram-positive bacteria, and Gram-negative bacteria) and SEQ ID NOs: 378-428 (mouse and rat). In some embodiments, a probe set comprises two or more, or five or more, or 10 or more, or 25 or more, or 44 sequences from SEQ ID NOs: 334-377 (Archaea). In some embodiments, a probe set comprises two or more, or five or more, or 10 or more, or 25 or more, or 50 or more, or 51 sequences from SEQ ID NOs: 378-428 (mouse and rat).

In some embodiments, the DNA probes are partially or completely complementary and comprise sequences that hybridize to human 28S, 18S, 5.8S and/or 5S rRNA, for example DNA probe sequences as shown in Table 1, SEQ ID NO: 40 through SEQ ID NO: 150. In a second embodiment, the DNA probes include sequences that hybridize to mitochondrial rRNAs 16S and/or 12S, for example DNA probe sequences as shown in Table 1, SEQ ID NO: 1 through SEQ ID NO: 39. In other embodiments, the DNA probes include sequences that hybridize to hemoglobin mRNA including HBA-A1, HBA-A2, HBB, HBB-B1, HBB-B2, HBG1, and/or HBG2, for example DNA probe sequences as shown in Table 1, SEQ ID NO: 151 through SEQ ID NO: 194. In some embodiments, the DNA probes include sequences that hybridize to bacterial rRNAs such as Gram positive and/or Gram negative bacterial rRNAs 23S, 16S and/or 5S, for example DNA probe sequences as shown in Table 1, SEQ ID NO: 195 through SEQ ID NO: 262 (Gram negative bacterial representative E. coli) and SEQ ID NO: 263 through SEQ ID NO: 333 (Gram positive bacterial representative Bacillus subtilis). In other embodiments, the DNA probes include sequences that hybridize to Archaea rRNAs, such as rRNAs 23S, 16S and/or 5S, for example the DNA probe sequences shown in Table 1, SEQ ID NO: 334 through SEQ ID NO: 384, which hybridize to rRNAs from Archaea species Methanobrevibacter smithii. In some embodiments, the DNA probes include sequences that hybridize to mouse rRNAs, such as mouse 16S and/or 28S, for example the DNA probe sequences shown in Table 1, SEQ ID NO: 385 through SEQ ID NO: 393 and SEQ ID NO:400 through SEQ ID NO: 419. In some embodiments, the DNA probes include sequences that hybridize to rat rRNAs, such as rat 16S and/or 28S, for example the DNA probe sequences shown in Table 1, SEQ ID NO: 394 through SEQ ID NO: 399 and SEQ ID NO: 420 through SEQ ID NO: 428.

TABLE 1
DNA probe sequences for unwanted RNA depletion

| SEQ ID | Probe | |
|---|---|---|
| NO | name | Probe sequence 5′-3′ |
| 1 | 12S_P1 | GTTCGTCCAAGTGCACTTTCCAGTACACTTACCATGTTACGACTTGTCTC |
| 2 | 12S_P2 | TAGGGGTTTTAGTTAAATGTCCTTTGAAGTATACTTGAGGAGGGTGACGG |
| 3 | 12S_P3 | TTCAGGGCCCTGTTCAACTAAGCACTCTACTCTCAGTTTACTGCTAAATC |
| 4 | 12S_P4 | AGTTTCATAAGGGCTATCGTAGTTTTCTGGGGTAGAAAATGTAGCCCATT |
| 5 | 12S_P5 | GGCTACACCTTGACCTAACGTCTTTACGTGGGTACTTGCGCTTACTTTGT |
| 6 | 12S_P6 | TTGCTGAAGATGGCGGTATATAGGCTGAGCAAGAGGTGGTGAGGTTGATC |
| 7 | 12S_P7 | CAGAACAGGCTCCTCTAGAGGGATATGAAGCACCGCCAGGTCCTTTGAGT |
| 8 | 12S_P8 | GTAGTGTTCTGGCGAGCAGTTTTGTTGATTTAACTGTTGAGGTTTAGGGC |
| 9 | 12S_P9 | ATCTAATCCCAGTTTGGGTCTTAGCTATTGTGTGTTCAGATATGTTAAAG |
| 10 | 12S_P10 | ATTTTGTGTCAACTGGAGTTTTTTACAACTCAGGTGAGTTTTAGCTTTAT |
| 11 | 12S_P11 | CTAAAACACTCTTTACGCCGGCTTCTATTGACTTGGGTTAATCGTGTGAC |
| 12 | 12S_P12 | GAAATTGACCAACCCTGGGGTTAGTATAGCTTAGTTAAACTTTCGTTTAT |
| 13 | 12S_P13 | ACTGCTGTTTCCCGTGGGGGTGTGGCTAGGCTAAGCGTTTTGAGCTGCAT |
| 14 | 12S_P14 | GCTTGTCCCTTTTGATCGTGGTGATTTAGAGGGTGAACTCACTGGAACGG |
| 15 | 12S_P15 | TAATCTTACTAAGAGCTAATAGAAAGGCTAGGACCAAACCTATTTGTTTA |
| 16 | 16S_P1 | AAACCCTGTTCTTGGGTGGGTGTGGGTATAATACTAAGTTGAGATGATAT |
| 17 | 16S_P2 | GCGCTTTGTGAAGTAGGCCTTATTTCTCTTGTCCTTTCGTACAGGGAGGA |
| 18 | 16S_P3 | AAACCGACCTGGATTACTCCGGTCTGAACTCAGATCACGTAGGACTTTAA |
| 19 | 16S_P4 | ACCTTTAATAGCGGCTGCACCATCGGGATGTCCTGATCCAACATCGAGGT |
| 20 | 16S_P5 | TGATATGGACTCTAGAATAGGATTGCGCTGTTATCCCTAGGGTAACTTGT |
| 21 | 16S_P6 | ATTGGATCAATTGAGTATAGTAGTTCGCTTTGACTGGTGAAGTCTTAGCA |
| 22 | 16S_P7 | TTGGGTTCTGCTCCGAGGTCGCCCCAACCGAAATTTTTAATGCAGGTTTG |
| 23 | 16S_P8 | TGGGTTTGTTAGGTACTGTTTGCATTAATAAATTAAAGCTCCATAGGGTC |
| 24 | 16S_P9 | GTCATGCCCGCCTCTTCACGGGCAGGTCAATTTCACTGGTTAAAAGTAAG |
| 25 | 16S_P10 | CGTGGAGCCATTCATACAGGTCCCTATTTAAGGAACAAGTGATTATGCTA |
| 26 | 16S_P11 | GGTACCGCGGCCGTTAAACATGTGTCACTGGGCAGGCGGTGCCTCTAATA |
| 27 | 16S_P12 | GTGATGTTTTTGGTAAACAGGCGGGGTAAGGTTTGCCGAGTTCCTTTTAC |
| 28 | 16S_P13 | CTTATGAGCATGCCTGTGTTGGGTTGACAGTGAGGGTAATAATGACTTGT |
| 29 | 16S_P14 | ATTGGGCTGTTAATTGTCAGTTCAGTGTTTTGATCTGACGCAGGCTTATG |
| 30 | 16S_P15 | TCATGTTACTTATACTAACATTAGTTCTTCTATAGGGTGATAGATTGGTC |
| 31 | 16S_P16 | AGTTCAGTTATATGTTTGGGATTTTTTAGGTAGTGGGTGTTGAGCTTGAA |
| 32 | 16S_P17 | TGGCTGCTTTTAGGCCTACTATGGGTGTTAAATTTTTTACTCTCTCTACA |
| 33 | 16S_P18 | GTCCAAAGAGCTGTTCCTCTTTGGACTAACAGTTAAATTTACAAGGGGAT |
| 34 | 16S_P19 | GGCAAATTTAAAGTTGAACTAAGATTCTATCTTGGACAACCAGCTATCAC |
| 35 | 16S_P20 | TGTCGCCTCTACCTATAAATCTTCCCACTATTTTGCTACATAGACGGGTG |
| 36 | 16S_P21 | TCTTAGGTAGCTCGTCTGGTTTCGGGGGTCTTAGCTTTGGCTCTCCTTGC |
| 37 | 16S_P22 | TAATTCATTATGCAGAAGGTATAGGGGTTAGTCCTTGCTATATTATGCTT |
| 38 | 16S_P23 | TCTTTCCCTTGCGGTACTATATCTATTGCGCCAGGTTTCAATTTCTATCG |
| 39 | 16S_P24 | GGTAAATGGTTTGGCTAAGGTTGTCTGGTAGTAAGGTGGAGTGGGTTTGG |
| 40 | 18S_P1 | TAATGATCCTTCCGCAGGTTCACCTACGGAAACCTTGTTACGACTTTTAC |
| 41 | 18S_P2 | AAGTTCGACCGTCTTCTCAGCGCTCCGCCAGGGCCGTGGGCCGACCCCGG |
| 42 | 18S_P3 | GGCCTCACTAAACCATCCAATCGGTAGTAGCGACGGGCGGTGTGTACAAA |
| 43 | 18S_P4 | CAACGCAAGCTTATGACCCGCACTTACTCGGGAATTCCCTCGTTCATGGG |
| 44 | 18S_P5 | CCGATCCCCATCACGAATGGGGTTCAACGGGTTACCCGCGCCTGCCGGCG |
| 45 | 18S_P6 | CTGAGCCAGTCAGTGTAGCGCGCGTGCAGCCCCGGACATCTAAGGGCATC |
| 46 | 18S_P7 | CTCAATCTCGGGTGGCTGAACGCCACTTGTCCCTCTAAGAAGTTGGGGGA |
| 47 | 18S_P8 | GGTCGCGTAACTAGTTAGCATGCCAGAGTCTCGTTCGTTATCGGAATTAA |
| 48 | 18S_P9 | CACCAACTAAGAACGGCCATGCACCACCACCCACGGAATCGAGAAAGAGC |
| 49 | 18S_P10 | CCTGTCCGTGTCCGGGCCGGGTGAGGTTTCCCGTGTTGAGTCAAATTAAG |
| 50 | 18S_P11 | CTGGTGGTGCCCTTCCGTCAATTCCTTTAAGTTTCAGCTTTGCAACCATA |
| 51 | 18S_P12 | AAAGACTTTGGTTTCCCGGAAGCTGCCCGGCGGGTCATGGGAATAACGCC |
| 52 | 18S_P13 | GGCATCGTTTATGGTCGGAACTACGACGGTATCTGATCGTCTTCGAACCT |
| 53 | 18S_P14 | GATTAATGAAAACATTCTTGGCAAATGCTTTCGCTCTGGTCCGTCTTGCG |
| 54 | 18S_P15 | CACCTCTAGCGGCGCAATACGAATGCCCCCGGCCGTCCCTCTTAATCATG |
| 55 | 18S_P16 | ACCAACAAAATAGAACCGCGGTCCTATTCCATTATTCCTAGCTGCGGTAT |
| 56 | 18S_P17 | CTGCTTTGAACACTCTAATTTTTTCAAAGTAAACGCTTCGGGCCCCGCGG |
| 57 | 18S_P18 | GCATCGAGGGGGCGCCGAGAGGCAAGGGGCGGGGACGGGCGGTGGCTCGC |
| 58 | 18S_P19 | CCGCCCGCTCCCAAGATCCAACTACGAGCTTTTTAACTGCAGCAACTTTA |
| 59 | 18S_P20 | GCTGGAATTACCGCGGCTGCTGGCACCAGACTTGCCCTCCAATGGATCCT |
| 60 | 18S_P21 | AGTGGACTCATTCCAATTACAGGGCCTCGAAAGAGTCCTGTATTGTTATT |
| 61 | 18S_P22 | CCCGGGTCGGGAGTGGGTAATTTGCGCGCCTGCTGCCTTCCTTGGATGTG |
| 62 | 18S_P23 | GCTCCCTCTCCGGAATCGAACCCTGATTCCCCGTCACCCGTGGTCACCAT |
| 63 | 18S_P24 | TACCATCGAAAGTTGATAGGGCAGACGTTCGAATGGGTCGTCGCCGCCAC |
| 64 | 18S_P25 | GGCCCGAGGTTATCTAGAGTCACCAAAGCCGCCGGCGCCCGCCCCCCGGC |
| 65 | 18S_P26 | GCTGACCGGGTTGGTTTTGATCTGATAAATGCACGCATCCCCCCCGCGAA |
| 66 | 18S_P27 | TCGGCATGTATTAGCTCTAGAATTACCACAGTTATCCAAGTAGGAGAGGA |
| 67 | 18S_P28 | AACCATAACTGATTTAATGAGCCATTCGCAGTTTCACTGTACCGGCCGTG |
| 68 | 18S_P29 | ATGGCTTAATCTTTGAGACAAGCATATGCTACTGGCAGGATCAACCAGGT |
| 69 | 28S_P1 | GACAAACCCTTGTGTCGAGGGCTGACTTTCAATAGATCGCAGCGAGGGAG |
| 70 | 28S_P2 | CGAAACCCCGACCCAGAAGCAGGTCGTCTACGAATGGTTTAGCGCCAGGT |
| 71 | 28S_P3 | GGTGCGTGACGGGCGAGGGGGCGGCCGCCTTTCCGGCCGCGCCCCGTTTC |
| 72 | 28S_P4 | CTCCGCACCGGACCCCGGTCCCGGCGCGCGGCGGGGCACGCGCCCTCCCG |
| 73 | 28S_P5 | AGGGGGGGGCGGCCCGCCGGCGGGGACAGGCGGGGGACCGGCTATCCGAG |
| 74 | 28S_P6 | GCGGCGCTGCCGTATCGTTCGCCTGGGCGGGATTCTGACTTAGAGGCGTT |
| 75 | 28S_P7 | AGATGGTAGCTTCGCCCCATTGGCTCCTCAGCCAAGCACATACACCAAAT |
| 76 | 28S_P8 | TCCTCTCGTACTGAGCAGGATTACCATGGCAACAACACATCATCAGTAGG |
| 77 | 28S_P9 | CTCACGACGGTCTAAACCCAGCTCACGTTCCCTATTAGTGGGTGAACAAT |
| 78 | 28S_P10 | TTCTGCTTCACAATGATAGGAAGAGCCGACATCGAAGGATCAAAAAGCGA |
| 79 | 28S_P11 | TTGGCCGCCACAAGCCAGTTATCCCTGTGGTAACTTTTCTGACACCTCCT |
| 80 | 28S_P12 | GGTCAGAAGGATCGTGAGGCCCCGCTTTCACGGTCTGTATTCGTACTGAA |
| 81 | 28S_P13 | AGCTTTTGCCCTTCTGCTCCACGGGAGGTTTCTGTCCTCCCTGAGCTCGC |
| 82 | 28S_P14 | TTACCGTTTGACAGGTGTACCGCCCCAGTCAAACTCCCCACCTGGCACTG |
| 83 | 28S_P15 | GCGCCCGGCCGGGCGGGCGCTTGGCGCCAGAAGCGAGAGCCCCTCGGGCT |
| 84 | 28S_P16 | CCGGGTCAGTGAAAAAACGATCAGAGTAGTGGTATTTCACCGGCGGCCCG |
| 85 | 28S_P17 | CGCCCCGGGCCCCTCGCGGGGACACCGGGGGGGCGCCGGGGGCCTCCCAC |
| 86 | 28S_P18 | CATGTCTCTTCACCGTGCCAGACTAGAGTCAAGCTCAACAGGGTCTTCTT |
| 87 | 28S_P19 | CCAAGCCCGTTCCCTTGGCTGTGGTTTCGCTGGATAGTAGGTAGGGACAG |
| 88 | 28S_P20 | TCCATTCATGCGCGTCACTAATTAGATGACGAGGCATTTGGCTACCTTAA |
| 89 | 28S_P21 | TCCCGCCGTTTACCCGCGCTTCATTGAATTTCTTCACTTTGACATTCAGA |
| 90 | 28S_P22 | CACATCGCGTCAACACCCGCCGCGGGCCTTCGCGATGCTTTGTTTTAATT |
| 91 | 28S_P23 | CCTGGTCCGCACCAGTTCTAAGTCGGCTGCTAGGCGCCGGCCGAGGCGAG |
| 92 | 28S_P24 | CGGCCCCGGGGGCGGACCCGGCGGGGGGGACCGGCCCGCGGCCCCTCCGC |
| 93 | 28S_P25 | CCGCCGCGCGCCGAGGAGGAGGGGGGAACGGGGGGCGGACGGGGCCGGGG |
| 94 | 28S_P26 | ACGAACCGCCCCGCCCCGCCGCCCGCCGACCGCCGCCGCCCGACCGCTCC |
| 95 | 28S_P27 | CGCGCGCGACCGAGACGTGGGGTGGGGGTGGGGGGCGCGCCGCGCCGCCG |
| 96 | 28S_P28 | GCGGCCGCGACGCCCGCCGCAGCTGGGGCGATCCACGGGAAGGGCCCGGC |
| 97 | 28S_P29 | GCGCCGCCGCCGGCCCCCCGGGTCCCCGGGGCCCCCCTCGCGGGGACCTG |
| 98 | 28S_P30 | CCGGCGGCCGCCGCGCGGCCCCTGCCGCCCCGACCCTTCTCCCCCCGCCG |
| 99 | 28S_P31 | CTCCCCCGGGGAGGGGGGAGGACGGGGAGCGGGGGAGAGAGAGAGAGAGA |
| 100 | 28S_P32 | AGGGAGCGAGCGGCGCGCGCGGGTGGGGCGGGGGAGGGCCGCGAGGGGGG |
| 101 | 28S_P33 | GGGGGCGCGCGCCTCGTCCAGCCGCGGCGCGCGCCCAGCCCCGCTTCGCG |
| 102 | 28S_P34 | CCCAGCCCTTAGAGCCAATCCTTATCCCGAAGTTACGGATCCGGCTTGCC |
| 103 | 28S_P35 | CATTGTTCCAACATGCCAGAGGCTGTTCACCTTGGAGACCTGCTGCGGAT |
| 104 | 28S_P36 | CGCGAGATTTACACCCTCTCCCCCGGATTTTCAAGGGCCAGCGAGAGCTC |
| 105 | 28S_P37 | AACCGCGACGCTTTCCAAGGCACGGGCCCCTCTCTCGGGGCGAACCCATT |
| 106 | 28S_P38 | CTTCACAAAGAAAAGAGAACTCTCCCCGGGGCTCCCGCCGGCTTCTCCGG |
| 107 | 28S_P39 | CGCACTGGACGCCTCGCGGCGCCCATCTCCGCCACTCCGGATTCGGGGAT |
| 108 | 28S_P40 | TTTCGATCGGCCGAGGGCAACGGAGGCCATCGCCCGTCCCTTCGGAACGG |
| 109 | 28S_P41 | CAGGACCGACTGACCCATGTTCAACTGCTGTTCACATGGAACCCTTCTCC |
| 110 | 28S_P42 | GTTCTCGTTTGAATATTTGCTACTACCACCAAGATCTGCACCTGCGGCGG |
| 111 | 28S_P43 | CGCCCTAGGCTTCAAGGCTCACCGCAGCGGCCCTCCTACTCGTCGCGGCG |
| 112 | 28S_P44 | TCCGGGGGCGGGGAGCGGGGCGTGGGCGGGAGGAGGGGAGGAGGCGTGGG |
| 113 | 28S_P45 | AGGACCCCACACCCCCGCCGCCGCCGCCGCCGCCGCCCTCCGACGCACAC |
| 114 | 28S_P46 | GCGCGCCGCCCCCGCCGCTCCCGTCCACTCTCGACTGCCGGCGACGGCCG |
| 115 | 28S_P47 | CTCCAGCGCCATCCATTTTCAGGGCTAGTTGATTCGGCAGGTGAGTTGTT |
| 116 | 28S_P48 | GATTCCGACTTCCATGGCCACCGTCCTGCTGTCTATATCAACCAACACCT |
| 117 | 28S_P49 | GAGCGTCGGCATCGGGCGCCTTAACCCGGCGTTCGGTTCATCCCGCAGCG |
| 118 | 28S_P50 | AAAAGTGGCCCACTAGGCACTCGCATTCCACGCCCGGCTCCACGCCAGCG |
| 119 | 28S_P51 | CCATTTAAAGTTTGAGAATAGGTTGAGATCGTTTCGGCCCCAAGACCTCT |
| 120 | 28S_P52 | CGGATAAAACTGCGTGGCGGGGGTGCGTCGGGTCTGCGAGAGCGCCAGCT |
| 121 | 28S_P53 | TCGGAGGGAACCAGCTACTAGATGGTTCGATTAGTCTTTCGCCCCTATAC |
| 122 | 28S_P54 | GATTTGCACGTCAGGACCGCTACGGACCTCCACCAGAGTTTCCTCTGGCT |
| 123 | 28S_P55 | ATAGTTCACCATCTTTCGGGTCCTAACACGTGCGCTCGTGCTCCACCTCC |
| 124 | 28S_P56 | AGACGGGCCGGTGGTGCGCCCTCGGCGGACTGGAGAGGCCTCGGGATCCC |
| 125 | 28S_P57 | CGCGCCGGCCTTCACCTTCATTGCGCCACGGCGGCTTTCGTGCGAGCCCC |
| 126 | 28S_P58 | TTAGACTCCTTGGTCCGTGTTTCAAGACGGGTCGGGTGGGTAGCCGACGT |
| 127 | 28S_P59 | GCGCTCGCTCCGCCGTCCCCCTCTTCGGGGGACGCGCGCGTGGCCCCGAG |
| 128 | 28S_P60 | CCCGACGGCGCGACCCGCCCGGGGCGCACTGGGGACAGTCCGCCCCGCCC |
| 129 | 28S_P61 | GCACCCCCCCCGTCGCCGGGGCGGGGGCGCGGGGAGGAGGGGTGGGAGAG |
| 130 | 28S_P62 | AGGGGTGGCCCGGCCCCCCCACGAGGAGACGCCGGCGCGCCCCCGCGGGG |
| 131 | 28S_P63 | GGGGATTCCCCGCGGGGGTGGGCGCCGGGAGGGGGGAGAGCGCGGCGACG |
| 132 | 28S_P64 | GCCCCGGGATTCGGCGAGTGCTGCTGCCGGGGGGGCTGTAACACTCGGGG |
| 133 | 28S_P65 | CCGCCCCCGCCGCCGCCGCCACCGCCGCCGCCGCCGCCGCCCCGACCCGC |
| 134 | 28S_P66 | AGGACGCGGGGCCGGGGGGCGGAGACGGGGGAGGAGGAGGACGGACGGAC |
| 135 | 28S_P67 | AGCCACCTTCCCCGCCGGGCCTTCCCAGCCGTCCCGGAGCCGGTCGCGGC |
| 136 | 28S_P68 | AAATGCGCCCGGCGGCGGCCGGTCGCCGGTCGGGGGACGGTCCCCCGCCG |
| 137 | 28S_P69 | CCGCCCGCCCACCCCCGCACCCGCCGGAGCCCGCCCCCTCCGGGGAGGAG |
| 138 | 28S_P70 | GGGAAGGGAGGGCGGGTGGAGGGGTCGGGAGGAACGGGGGGCGGGAAAGA |
| 139 | 28S_P71 | ACACGGCCGGACCCGCCGCCGGGTTGAATCCTCCGGGCGGACTGCGCGGA |
| 140 | 28S_P72 | TCTTAACGGTTTCACGCCCTCTTGAACTCTCTCTTCAAAGTTCTTTTCAA |
| 141 | 28S_P73 | CTTGTTGACTATCGGTCTCGTGCCGGTATTTAGCCTTAGATGGAGTTTAC |
| 142 | 28S_P74 | GCATTCCCAAGCAACCCGACTCCGGGAAGACCCGGGCGCGCGCCGGCCGC |
| 143 | 28S_P75 | GTCCACGGGCTGGGCCTCGATCAGAAGGACTTGGGCCCCCCACGAGCGGC |
| 144 | 28S_P76 | TTCCGTACGCCACATGTCCCGCGCCCCGCGGGGCGGGGATTCGGCGCTGG |
| 145 | 28S_P77 | CTCGCCGTTACTGAGGGAATCCTGGTTAGTTTCTTTTCCTCCGCTGACTA |
| 146 | 28S_P78 | GCGGGTCGCCACGTCTGATCTGAGGTCGCGTCTCGGAGGGGGACGGGCCG |
| 147 | 5.8S_P1 | AAGCGACGCTCAGACAGGCGTAGCCCCGGGAGGAACCCGGGGCCGCAAGT |
| 148 | 5.8S_P3 | GCAGCTAGCTGCGTTCTTCATCGACGCACGAGCCGAGTGATCCACCGCTA |
| 149 | 5S_P1 | AAAGCCTACAGCACCCGGTATTCCCAGGCGGTCTCCCATCCAAGTACTAA |
| 150 | 5S_P3 | TTCCGAGATCAGACGAGATCGGGCGCGTTCAGGGTGGTATGGCCGTAGAC |
| 151 | HBA1_P1 | GCCGCCCACTCAGACTTTATTCAAAGACCACGGGGGTACGGGTGCAGGAA |
| 152 | HBA1_P2 | GGGGGAGGCCCAAGGGGCAAGAAGCATGGCCACCGAGGCTCCAGCTTAAC |
| 153 | HBA1_P3 | GCACGGTGCTCACAGAAGCCAGGAACTTGTCCAGGGAGGCGTGCACCGCA |
| 154 | HBA1_P4 | GGGAGGTGGGCGGCCAGGGTCACCAGCAGGCAGTGGCTTAGGAGCTTGAA |
| 155 | HBA1_P5 | CCGAAGCTTGTGCGCGTGCAGGTCGCTCAGGGCGGACAGCGCGTTGGGCA |
| 156 | HBA1_P6 | CCACGGCGTTGGTCAGCGCGTCGGCCACCTTCTTGCCGTGGCCCTTAACC |
| 157 | HBA1_P7 | CTCAGGTCGAAGTGCGGGAAGTAGGTCTTGGTGGTGGGGAAGGACAGGAA |
| 158 | HBA1_P8 | CTCCGCACCATACTCGCCAGCGTGCGCGCCGACCTTACCCCAGGCGGCCT |
| 159 | HBA1_P9 | CGGCAGGAGACAGCACCATGGTGGGTTCTCTCTGAGTCTGTGGGGACCAG |
| 160 | HBA2_P1 | GAGGGGAGGAGGGCCCGTTGGGAGGCCCAGCGGGCAGGAGGAACGGCTAC |
| 161 | HBA2_P2 | ACGGTATTTGGAGGTCAGCACGGTGCTCACAGAAGCCAGGAACTTGTCCA |
| 162 | HBA2_P3 | CAGGGGTGAACTCGGCGGGGAGGTGGGCGGCCAGGGTCACCAGCAGGCAG |
| 163 | HBA2_P4 | AAGTTGACCGGGTCCACCCGAAGCTTGTGCGCGTGCAGGTCGCTCAGGGC |
| 164 | HBA2_P5 | CATGTCGTCCACGTGCGCCACGGCGTTGGTCAGCGCGTCGGCCACCTTCT |
| 165 | HBA2_P6 | CCTGGGCAGAGCCGTGGCTCAGGTCGAAGTGCGGGAAGTAGGTCTTGGTG |
| 166 | HBA2_P7 | AACATCCTCTCCAGGGCCTCCGCACCATACTCGCCAGCGTGCGCGCCGAC |
| 167 | HBA2_P8 | CTTGACGTTGGTCTTGTCGGCAGGAGACAGCACCATGGTGGGTTCTCTCT |
| 168 | HBB_P1 | GCAATGAAAATAAATGTTTTTTATTAGGCAGAATCCAGATGCTCAAGGCC |
| 169 | HBB_P2 | CAGTTTAGTAGTTGGACTTAGGGAACAAAGGAACCTTTAATAGAAATTGG |
| 170 | HBB_P3 | GCTTAGTGATACTTGTGGGCCAGGGCATTAGCCACACCAGCCACCACTTT |
| 171 | HBB_P4 | CACTGGTGGGGTGAATTCTTTGCCAAAGTGATGGGCCAGCACACAGACCA |
| 172 | HBB_P5 | GCCTGAAGTTCTCAGGATCCACGTGCAGCTTGTCACAGTGCAGCTCACTC |
| 173 | HBB_P6 | CCCTTGAGGTTGTCCAGGTGAGCCAGGCCATCACTAAAGGCACCGAGCAC |
| 174 | HBB_P7 | CTTCACCTTAGGGTTGCCCATAACAGCATCAGGAGTGGACAGATCCCCAA |
| 175 | HBB_P8 | TCTGGGTCCAAGGGTAGACCACCAGCAGCCTGCCCAGGGCCTCACCACCA |
| 176 | HBB_P9 | ACCTTGCCCCACAGGGCAGTAACGGCAGACTTCTCCTCAGGAGTCAGATG |
| 177 | HBG1_P1 | GTGATCTCTCAGCAGAATAGATTTATTATTTGTATTGCTTGCAGAATAAA |
| 178 | HBG1_P2 | CTCTGAATCATGGGCAGTGAGCTCAGTGGTATCTGGAGGACAGGGCACTG |
| 179 | HBG1_P3 | ATCTTCTGCCAGGAAGCCTGCACCTCAGGGGTGAATTCTTTGCCGAAATG |
| 180 | HBG1_P4 | CACCAGCACATTTCCCAGGAGCTTGAAGTTCTCAGGATCCACATGCAGCT |
| 181 | HBG1_P5 | CACTCAGCTGGGCAAAGGTGCCCTTGAGATCATCCAGGTGCTTTGTGGCA |
| 182 | HBG1_P6 | AGCACCTTCTTGCCATGTGCCTTGACTTTGGGGTTGCCCATGATGGCAGA |
| 183 | HBG1_P7 | GCCAAAGCTGTCAAAGAACCTCTGGGTCCATGGGTAGACAACCAGGAGCC |
| 184 | HBG1_P8 | CTCCAGCATCTTCCACATTCACCTTGCCCCACAGGCTTGTGATAGTAGCC |
| 185 | HBG1_P9 | AAATGACCCATGGCGTCTGGACTAGGAGCTTATTGATAACCTCAGACGTT |
| 186 | HBG2_P1 | GTGATCTCTTAGCAGAATAGATTTATTATTTGATTGCTTGCAGAATAAAG |
| 187 | HBG2_P2 | TCTGCATCATGGGCAGTGAGCTCAGTGGTATCTGGAGGACAGGGCACTGG |
| 188 | HBG2_P3 | TCTTCTGCCAGGAAGCCTGCACCTCAGGGGTGAATTCTTTGCCGAAATGG |
| 189 | HBG2_P4 | ACCAGCACATTTCCCAGGAGCTTGAAGTTCTCAGGATCCACATGCAGCTT |
| 190 | HBG2_P5 | ACTCAGCTGGGCAAAGGTGCCCTTGAGATCATCCAGGTGCTTTATGGCAT |
| 191 | HBG2_P6 | GCACCTTCTTGCCATGTGCCTTGACTTTGGGGTTGCCCATGATGGCAGAG |
| 192 | HBG2_P7 | CCAAAGCTGTCAAAGAACCTCTGGGTCCATGGGTAGACAACCAGGAGCCT |
| 193 | HBG2_P8 | TCCAGCATCTTCCACATTCACCTTGCCCCACAGGCTTGTGATAGTAGCCT |
| 194 | HBG2_P9 | AATGACCCATGGCGTCTGGACTAGGAGCTTATTGATAACCTCAGACGTTC |
| 195 | 5S_GNbac_P1 | ATGCCTGGCAGTTCCCTACTCTCGCATGGGGAGACCCCACACTACCATCG |
| 196 | 5S_GNbac_P2 | ACTTCTGAGTTCGGCATGGGGTCAGGTGGGACCACCGCGCTACGGCCGCC |
| 197 | 16S_GNbac_P1 | GGTTACCTTGTTACGACTTCACCCCAGTCATGAATCACAAAGTGGTAAGT |
| 198 | 16S_GNbac_P2 | AAGCTACCTACTTCTTTTGCAACCCACTCCCATGGTGTGACGGGCGGTGT |
| 199 | 16S_GNbac_P3 | ACGTATTCACCGTGGCATTCTGATCCACGATTACTAGCGATTCCGACTTC |
| 200 | 16S_GNbac_P4 | AGACTCCAATCCGGACTACGACGCACTTTATGAGGTCCGCTTGCTCTCGC |
| 201 | 16S_GNbac_P5 | TGTATGCGCCATTGTAGCACGTGTGTAGCCCTGGTCGTAAGGGCCATGAT |
| 202 | 16S_GNbac_P6 | CCACCTTCCTCCAGTTTATCACTGGCAGTCTCCTTTGAGTTCCCGGCCGG |
| 203 | 16S_GNbac_P7 | GGATAAGGGTTGCGCTCGTTGCGGGACTTAACCCAACATTTCACAACACG |
| 204 | 16S_GNbac_P8 | TGCAGCACCTGTCTCACGGTTCCCGAAGGCACATTCTCATCTCTGAAAAC |
| 205 | 16S_GNbac_P9 | GACCAGGTAAGGTTCTTCGCGTTGCATCGAATTAAACCACATGCTCCACC |
| 206 | 16S_GNbac_P10 | CGTCAATTCATTTGAGTTTTAACCTTGCGGCCGTACTCCCCAGGCGGTCG |
| 207 | 16S_GNbac_P11 | TCCGGAAGCCACGCCTCAAGGGCACAACCTCCAAGTCGACATCGTTTACG |
| 208 | 16S_GNbac_P12 | GTATCTAATCCTGTTTGCTCCCCACGCTTTCGCACTGAGCGTCAGTCTTC |
| 209 | 16S_GNbac_P13 | TTCGCCACCGGTATTCCTCCAGATCTCTACGCATTTCACCGCTACACCTG |
| 210 | 16S_GNbac_P14 | CTACGAGACTCAAGCTTGCCAGTATCAGATGCAGTTCCCAGGTTGAGCCC |
| 211 | 16S_GNbac_P15 | GACTTAACAAACCGCCTGCGTGCGCTTTACGCCCAGTAATTCCGATTAAC |
| 212 | 16S_GNbac_P16 | ATTACCGCGGCTGCTGGCACGGAGTTAGCCGGTGCTTCTTCTGCGGGTAA |
| 213 | 16S_GNbac_P17 | GTATTAACTTTACTCCCTTCCTCCCCGCTGAAAGTACTTTACAACCCGAA |
| 214 | 16S_GNbac_P18 | CGCGGCATGGCTGCATCAGGCTTGCGCCCATTGTGCAGTATTCCCCACTG |
| 215 | 16S_GNbac_P19 | GTCTGGACCGTGTCTCAGTTCCAGTGTGGCTGGTCATCCTCTCAGACCAG |
| 216 | 16S_GNbac_P20 | TAGGTGAGCCGTTACCCCACCTACTAGCTAATCCCATCTGGGCACATCCG |
| 217 | 16S_GNbac_P21 | AAGGTCCCCCTCTTTGGTCTTGCGACGTTATGCGGTATTAGCTACCGTTT |
| 218 | 16S_GNbac_P22 | CTCCATCAGGCAGTTTCCCAGACATTACTCACCCGTCCGCCACTCGTCAG |
| 219 | 23S_GNbac_P1 | AAGGTTAAGCCTCACGGTTCATTAGTACCGGTTAGCTCAACGCATCGCTG |
| 220 | 23S_GNbac_P2 | CCTATCAACGTCGTCGTCTTCAACGTTCCTTCAGGACCCTTAAAGGGTCA |
| 221 | 23S_GNbac_P3 | GGGGCAAGTTTCGTGCTTAGATGCTTTCAGCACTTATCTCTTCCGCATTT |
| 222 | 23S_GNbac_P4 | CCATTGGCATGACAACCCGAACACCAGTGATGCGTCCACTCCGGTCCTCT |
| 223 | 23S_GNbac_P5 | CCCCCTCAGTTCTCCAGCGCCCACGGCAGATAGGGACCGAACTGTCTCAC |
| 224 | 23S_GNbac_P6 | GCTCGCGTACCACTTTAAATGGCGAACAGCCATACCCTTGGGACCTACTT |
| 225 | 23S_GNbac_P7 | ATGAGCCGACATCGAGGTGCCAAACACCGCCGTCGATATGAACTCTTGGG |
| 226 | 23S_GNbac_P8 | ATCCCCGGAGTACCTTTTATCCGTTGAGCGATGGCCCTTCCATTCAGAAC |
| 227 | 23S_GNbac_P9 | ACCTGCTTTCGCACCTGCTCGCGCCGTCACGCTCGCAGTCAAGCTGGCTT |
| 228 | 23S_GNbac_P10 | CCTCCTGATGTCCGACCAGGATTAGCCAACCTTCGTGCTCCTCCGTTACT |
| 229 | 23S_GNbac_P11 | GCCCCAGTCAAACTACCCACCAGACACTGTCCGCAACCCGGATTACGGGT |
| 230 | 23S_GNbac_P12 | AAACATTAAAGGGTGGTATTTCAAGGTCGGCTCCATGCAGACTGGCGTCC |
| 231 | 23S_GNbac_P13 | CCACCTATCCTACACATCAAGGCTCAATGTTCAGTGTCAAGCTATAGTAA |
| 232 | 23S_GNbac_P14 | TTCCGTCTTGCCGCGGGTACACTGCATCTTCACAGCGAGTTCAATTTCAC |
| 233 | 23S_GNbac_P15 | GACAGCCTGGCCATCATTACGCCATTCGTGCAGGTCGGAACTTACCCGAC |
| 234 | 23S_GNbac_P16 | CTTAGGACCGTTATAGTTACGGCCGCCGTTTACCGGGGCTTCGATCAAGA |
| 235 | 23S_GNbac_P17 | ACCCCATCAATTAACCTTCCGGCACCGGGCAGGCGTCACACCGTATACGT |
| 236 | 23S_GNbac_P18 | CACAGTGCTGTGTTTTTAATAAACAGTTGCAGCCAGCTGGTATCTTCGAC |
| 237 | 23S_GNbac_P19 | CCGCGAGGGACCTCACCTACATATCAGCGTGCCTTCTCCCGAAGTTACGG |
| 238 | 23S_GNbac_P20 | TTCCTTCACCCGAGTTCTCTCAAGCGCCTTGGTATTCTCTACCTGACCAC |
| 239 | 23S_GNbac_P21 | GTACGATTTGATGTTACCTGATGCTTAGAGGCTTTTCCTGGAAGCAGGGC |
| 240 | 23S_GNbac_P22 | ACCGTAGTGCCTCGTCATCACGCCTCAGCCTTGATTTTCCGGATTTGCCT |
| 241 | 23S_GNbac_P23 | ACGCTTAAACCGGGACAACCGTCGCCCGGCCAACATAGCCTTCTCCGTCC |
| 242 | 23S_GNbac_P24 | ACCAAGTACAGGAATATTAACCTGTTTCCCATCGACTACGCCTTTCGGCC |
| 243 | 23S_GNbac_P25 | ACTCACCCTGCCCCGATTAACGTTGGACAGGAACCCTTGGTCTTCCGGCG |
| 244 | 23S_GNbac_P26 | CGCTTTATCGTTACTTATGTCAGCATTCGCACTTCTGATACCTCCAGCAT |
| 245 | 23S_GNbac_P27 | TTCGCAGGCTTACAGAACGCTCCCCTACCCAACAACGCATAAGCGTCGCT |
| 246 | 23S_GNbac_P28 | CATGGTTTAGCCCCGTTACATCTTCCGCGCAGGCCGACTCGACCAGTGAG |
| 247 | 23S_GNbac_P29 | TAAATGATGGCTGCTTCTAAGCCAACATCCTGGCTGTCTGGGCCTTCCCA |
| 248 | 23S_GNbac_P30 | AACCATGACTTTGGGACCTTAGCTGGCGGTCTGGGTTGTTTCCCTCTTCA |
| 249 | 23S_GNbac_P31 | CCCGCCGTGTGTCTCCCGTGATAACATTCTCCGGTATTCGCAGTTTGCAT |
| 250 | 23S_GNbac_P32 | GGATGACCCCCTTGCCGAAACAGTGCTCTACCCCCGGAGATGAATTCACG |
| 251 | 23S_GNbac_P33 | AGCTTTCGGGGAGAACCAGCTATCTCCCGGTTTGATTGGCCTTTCACCCC |
| 252 | 23S_GNbac_P34 | CGCTAATTTTTCAACATTAGTCGGTTCGGTCCTCCAGTTAGTGTTACCCA |
| 253 | 23S_GNbac_P35 | ATGGCTAGATCACCGGGTTTCGGGTCTATACCCTGCAACTTAACGCCCAG |
| 254 | 23S_GNbac_P36 | CCTTCGGCTCCCCTATTCGGTTAACCTTGCTACAGAATATAAGTCGCTGA |
| 255 | 23S_GNbac_P37 | GTACGCAGTCACACGCCTAAGCGTGCTCCCACTGCTTGTACGTACACGGT |
| 256 | 23S_GNbac_P38 | ACTCCCCTCGCCGGGGTTCTTTTCGCCTTTCCCTCACGGTACTGGTTCAC |
| 257 | 23S_GNbac_P39 | AGTATTTAGCCTTGGAGGATGGTCCCCCCATATTCAGACAGGATACCACG |
| 258 | 23S_GNbac_P40 | ATCGAGCTCACAGCATGTGCATTTTTGTGTACGGGGCTGTCACCCTGTAT |
| 259 | 23S_GNbac_P41 | ACGCTTCCACTAACACACACACTGATTCAGGCTCTGGGCTGCTCCCCGTT |
| 260 | 23S_GNbac_P42 | GGGGAATCTCGGTTGATTTCTTTTCCTCGGGGTACTTAGATGTTTCAGTT |
| 261 | 23S_GNbac_P43 | ATTAACCTATGGATTCAGTTAATGATAGTGTGTCGAAACACACTGGGTTT |
| 262 | 23S_GNbac_P44 | GCCGGTTATAACGGTTCATATCACCTTACCGACGCTTATCGCAGATTAGC |
| 263 | 5S_GPbac_P1 | GCTTGGCGGCGTCCTACTCTCACAGGGGGAAACCCCCGACTACCATCGGC |
| 264 | 5S_GPbac_P2 | TTCCGTGTTCGGTATGGGAACGGGTGTGACCTCTTCGCTATCGCCACCAA |
| 265 | 16S_GPbac_P1 | TAGAAAGGAGGTGATCCAGCCGCACCTTCCGATACGGCTACCTTGTTACG |
| 266 | 16S_GPbac_P2 | TCTGTCCCACCTTCGGCGGCTGGCTCCTAAAAGGTTACCTCACCGACTTC |
| 267 | 16S_GPbac_P3 | TCGTGGTGTGACGGGCGGTGTGTACAAGGCCCGGGAACGTATTCACCGCG |
| 268 | 16S_GPbac_P4 | ATTACTAGCGATTCCAGCTTCACGCAGTCGAGTTGCAGACTGCGATCCGA |
| 269 | 16S_GPbac_P5 | GTGGGATTGGCTTAACCTCGCGGTTTCGCTGCCCTTTGTTCTGTCCATTG |
| 270 | 16S_GPbac_P6 | CCAGGTCATAAGGGGCATGATGATTTGACGTCATCCCCACCTTCCTCCGG |
| 271 | 16S_GPbac_P7 | CACCTTAGAGTGCCCAACTGAATGCTGGCAACTAAGATCAAGGGTTGCGC |
| 272 | 16S_GPbac_P8 | ACCCAACATCTCACGACACGAGCTGACGACAACCATGCACCACCTGTCAC |
| 273 | 16S_GPbac_P9 | GACGTCCTATCTCTAGGATTGTCAGAGGATGTCAAGACCTGGTAAGGTTC |
| 274 | 16S_GPbac_P10 | ATTAAACCACATGCTCCACCGCTTGTGCGGGCCCCCGTCAATTCCTTTGA |
| 275 | 16S_GPbac_P11 | CCGTACTCCCCAGGCGGAGTGCTTAATGCGTTAGCTGCAGCACTAAGGGG |
| 276 | 16S_GPbac_P12 | ACTTAGCACTCATCGTTTACGGCGTGGACTACCAGGGTATCTAATCCTGT |
| 277 | 16S_GPbac_P13 | TCGCTCCTCAGCGTCAGTTACAGACCAGAGAGTCGCCTTCGCCACTGGTG |
| 278 | 16S_GPbac_P14 | ACGCATTTCACCGCTACACGTGGAATTCCACTCTCCTCTTCTGCACTCAA |
| 279 | 16S_GPbac_P15 | ATGACCCTCCCCGGTTGAGCCGGGGGCTTTCACATCAGACTTAAGAAACC |
| 280 | 16S_GPbac_P16 | ACGCCCAATAATTCCGGACAACGCTTGCCACCTACGTATTACCGCGGCTG |
| 281 | 16S_GPbac_P17 | CCGTGGCTTTCTGGTTAGGTACCGTCAAGGTACCGCCCTATTCGAACGGT |
| 282 | 16S_GPbac_P18 | ACAACAGAGCTTTACGATCCGAAAACCTTCATCACTCACGCGGCGTTGCT |
| 283 | 16S_GPbac_P19 | CCATTGCGGAAGATTCCCTACTGCTGCCTCCCGTAGGAGTCTGGGCCGTG |
| 284 | 16S_GPbac_P20 | GGCCGATCACCCTCTCAGGTCGGCTACGCATCGTCGCCTTGGTGAGCCGT |
| 285 | 16S_GPbac_P21 | CTAATGCGCCGCGGGTCCATCTGTAAGTGGTAGCCGAAGCCACCTTTTAT |
| 286 | 16S_GPbac_P22 | TTCAAACAACCATCCGGTATTAGCCCCGGTTTCCCGGAGTTATCCCAGTC |
| 287 | 16S_GPbac_P23 | CCACGTGTTACTCACCCGTCCGCCGCTAACATCAGGGAGCAAGCTCCCAT |
| 288 | 16S_GPbac_P24 | GCATGTATTAGGCACGCCGCCAGCGTTCGTCCTGAGCCAGGATCAAACTC |
| 289 | 23S_GPbac_P1 | TGGTTAAGTCCTCGATCGATTAGTATCTGTCAGCTCCATGTGTCGCCACA |
| 290 | 23S_GPbac_P2 | TATCAACCTGATCATCTTTCAGGGATCTTACTTCCTTGCGGAATGGGAAA |
| 291 | 23S_GPbac_P3 | GGCTTCATGCTTAGATGCTTTCAGCACTTATCCCGTCCGCACATAGCTAC |
| 292 | 23S_GPbac_P4 | GCAGAACAACTGGTACACCAGCGGTGCGTCCATCCCGGTCCTCTCGTACT |
| 293 | 23S_GPbac_P5 | CAAATTTCCTGCGCCCGCGACGGATAGGGACCGAACTGTCTCACGACGTT |
| 294 | 23S_GPbac_P6 | GTACCGCTTTAATGGGCGAACAGCCCAACCCTTGGGACTGACTACAGCCC |
| 295 | 23S_GPbac_P7 | CGACATCGAGGTGCCAAACCTCCCCGTCGATGTGGACTCTTGGGGGAGAT |
| 296 | 23S_GPbac_P8 | GGGGTAGCTTTTATCCGTTGAGCGATGGCCCTTCCATGCGGAACCACCGG |
| 297 | 23S_GPbac_P9 | TTTCGTCCCTGCTCGACTTGTAGGTCTCGCAGTCAAGCTCCCTTGTGCCT |
| 298 | 23S_GPbac_P10 | GATTTCCAACCATTCTGAGGGAACCTTTGGGCGCCTCCGTTACCTTTTAG |
| 299 | 23S_GPbac_P11 | GTCAAACTGCCCACCTGACACTGTCTCCCCGCCCGATAAGGGCGGCGGGT |
| 300 | 23S_GPbac_P12 | GCCAGGGTAGTATCCCACCGATGCCTCCACCGAAGCTGGCGCTCCGGTTT |
| 301 | 23S_GPbac_P13 | ATCCTGTACAAGCTGTACCAACATTCAATATCAGGCTGCAGTAAAGCTCC |
| 302 | 23S_GPbac_P14 | CCTGTCGCGGGTAACCTGCATCTTCACAGGTACTATAATTTCACCGAGTC |
| 303 | 23S_GPbac_P15 | GCCCAGATCGTTGCGCCTTTCGTGCGGGTCGGAACTTACCCGACAAGGAA |
| 304 | 23S_GPbac_P16 | ACCGTTATAGTTACGGCCGCCGTTTACTGGGGCTTCAATTCGCACCTTCG |
| 305 | 23S_GPbac_P17 | CCTCTTAACCTTCCAGCACCGGGCAGGCGTCAGCCCCTATACTTCGCCTT |
| 306 | 23S_GPbac_P18 | CCTGTGTTTTTGCTAAACAGTCGCCTGGGCCTATTCACTGCGGCTCTCTC |
| 307 | 23S_GPbac_P19 | CAGAGCACCCCTTCTCCCGAAGTTACGGGGTCATTTTGCCGAGTTCCTTA |
| 308 | 23S_GPbac_P20 | ATCACCTTAGGATTCTCTCCTCGCCTACCTGTGTCGGTTTGCGGTACGGG |
| 309 | 23S_GPbac_P21 | TAGAGGCTTTTCTTGGCAGTGTGGAATCAGGAACTTCGCTACTATATTTC |
| 310 | 23S_GPbac_P22 | TCAGCCTTATGGGAAACGGATTTGCCTATTTCCCAGCCTAACTGCTTGGA |
| 311 | 23S_GPbac_P23 | CCGCGCTTACCCTATCCTCCTGCGTCCCCCCATTGCTCAAATGGTGAGGA |
| 312 | 23S_GPbac_P24 | TCAACCTGTTGTCCATCGCCTACGCCTTTCGGCCTCGGCTTAGGTCCCGA |
| 313 | 23S_GPbac_P25 | CGAGCCTTCCTCAGGAAACCTTAGGCATTCGGTGGAGGGGATTCTCACCC |
| 314 | 23S_GPbac_P26 | TACCGGCATTCTCACTTCTAAGCGCTCCACCAGTCCTTCCGGTCTGGCTT |
| 315 | 23S_GPbac_P27 | GCTCTCCTACCACTGTTCGAAGAACAGTCCGCAGCTTCGGTGATACGTTT |
| 316 | 23S_GPbac_P28 | TCGGCGCAGAGTCACTCGACCAGTGAGCTATTACGCACTCTTTAAATGGT |
| 317 | 23S_GPbac_P29 | AACATCCTGGTTGTCTAAGCAACTCCACATCCTTTTCCACTTAACGTATA |
| 318 | 23S_GPbac_P30 | TGGCGGTCTGGGCTGTTTCCCTTTCGACTACGGATCTTATCACTCGCAGT |
| 319 | 23S_GPbac_P31 | AAGTCATTGGCATTCGGAGTTTGACTGAATTCGGTAACCCGGTAGGGGCC |
| 320 | 23S_GPbac_P32 | GCTCTACCTCCAAGACTCTTACCTTGAGGCTAGCCCTAAAGCTATTTCGG |
| 321 | 23S_GPbac_P33 | TCCAGGTTCGATTGGCATTTCACCCCTACCCACACCTCATCCCCGCACTT |
| 322 | 23S_GPbac_P34 | TTCGGGCCTCCATTCAGTGTTACCTGAACTTCACCCTGGACATGGGTAGA |
| 323 | 23S_GPbac_P35 | TCTACGACCACGTACTCATGCGCCCTATTCAGACTCGCTTTCGCTGCGGC |
| 324 | 23S_GPbac_P36 | TAACCTTGCACGGGATCGTAACTCGCCGGTTCATTCTACAAAAGGCACGC |
| 325 | 23S_GPbac_P37 | GGCTCTGACTACTTGTAGGCACACGGTTTCAGGATCTCTTTCACTCCCCT |
| 326 | 23S_GPbac_P38 | ACCTTTCCCTCACGGTACTGGTTCACTATCGGTCACTAGGGAGTATTTAG |
| 327 | 23S_GPbac_P39 | CTCCCGGATTCCGACGGAATTTCACGTGTTCCGCCGTACTCAGGATCCAC |
| 328 | 23S_GPbac_P40 | GTTTTGACTACAGGGCTGTTACCTCCTATGGCGGGCCTTTCCAGACCTCT |
| 329 | 23S_GPbac_P41 | CTTTGTAACTCCGTACAGAGTGTCCTACAACCCCAAGAGGCAAGCCTCTT |
| 330 | 23S_GPbac_P42 | CGTTTCGCTCGCCGCTACTCAGGGAATCGCATTTGCTTTCTCTTCCTCCG |
| 331 | 23S_GPbac_P43 | CAGTTCCCCGGGTCTGCCTTCTCATATCCTATGAATTCAGATATGGATAC |
| 332 | 23S_GPbac_P44 | GGTGGGTTTCCCCATTCGGAAATCTCCGGATCAAAGCTTGCTTACAGCTC |
| 333 | 23S_GPbac_P45 | TGTTCGTCCCGTCCTTCATCGGCTCCTAGTGCCAAGGCATCCACCGTGCG |
| 334 | 16S: A1 | AAACTAGATTCGAATATAACAAAACATTACATCCTCATCCAATCCCTTTT |
| 335 | 16S: A2 | GCGGTGTGTGCAAGGAGCAGGGACGTATTCACCGCGCGATTGTGACACGC |
| 336 | 16S: A3 | GCCTTTCGGCGTCGGAACCCATTGTCTCAGCCATTGTAGCCCGCGTGTTG |
| 337 | 16S: A4 | GCATACGGACCTACCGTCGTCCACTCCTTCCTCCTATTTATCATAGGCGG |
| 338 | 16S: A5 | CGGCATCCAAAAAAGGATCCGCTGGTAACTAAGAGCGTGGGTCTCGCTCG |
| 339 | 16S: A6 | CAACCTGGCTATCATACAGCTGTCGCCTCTGGTGAGATGTCCGGCGTTGA |
| 340 | 16S: A7 | AGGCTCCACGCGTTGTGGTGCTCCCCCGCCAATTCCTTTAAGTTTCAGTC |
| 341 | 16S: A8 | CCAGGCGGCGGACTTAACAGCTTCCCTTCGGCACTGGGACAGCTCAAAGC |
| 342 | 16S: A9 | TCCGCATCGTTTACAGCTAGGACTACCCGGGTATCTAATCCGGTTCGCGC |
| 343 | 16S: A10 | TTCCCACAGTTAAGCTGCAGGATTTCACCAGAGACTTATTAAACCGGCTA |
| 344 | 16S: A12 | CTCTTATTCCAAAAGCTCTTTACACTAATGAAAAGCCATCCCGTTAAGAA |
| 345 | 16S: A13 | CCCCCGTCGCGATTTCTCACATTGCGGAGGTTTCGCGCCTGCTGCACCCC |
| 346 | 16S: A14 | TTGTCTCAGGTTCCATCTCCGGGCTCTTGCTCTCACAACCCGTACCGATC |
| 347 | 16S: A16 | CATTACCTAACCAACTACCTAATCGGCCGCAGACCCATCCTTAGGCGAAA |
| 348 | 16S: A17 | AAACCATTACAGGAATAATTGCCTATCCAGTATTATCCCCAGTTTCCCAG |
| 349 | 16S: A18 | AAGGGTAGGTTATCCACGTGTTACTGAGCCGTACGCCACGAGCCTAAACT |
| 350 | 23S: A1 | ACCTAGCGCGTAGCTGCCCGGCACTGCCTTATCAGACAACCGGTCGACCA |
| 351 | 23S: A2 | CGTTCCTCTCGTACTGGAGCCACCTTCCCCTCAGACTACTAACACATCCA |
| 352 | 23S: A3 | CCTGTCTCACGACGGTCTAAACCCAGCTCACGTTCCCCTTTAATGGGCGA |
| 353 | 23S: A4 | GGTGCTGCTGCACACCCAGGATGGAAAGAACCGACATCGAAGTAGCAAGC |
| 354 | 23S: A5 | GGCTCTTGCCTGCGACCACCCAGTTATCCCCGAGGTAGTTTTTCTGTCAT |
| 355 | 23S: A6 | AGGAGGACTCTGAGGTTCGCTAGGCCCGGCTTTCGCCTCTGGATTTCTTG |
| 356 | 23S: A7 | CAAAGTAAGTTAGAAACACAGTCATAAGAAAGTGGTGTCTCAAGAACGAA |
| 357 | 23S: A8 | GACTTATAATCGAATTCTCCCACTTACACTGCATACCTATAACCAAGCTT |
| 358 | 23S: A9 | GTAAAACTCTACGGGGTCTTCGCTTCCCAATGGAAGACTCTGGCTTGTGC |
| 359 | 23S: A10 | TCACTAAGTTCTAGCTAGGGACAGTGGGGACCTCGTTCTACCATTCATGC |
| 360 | 23S: A11 | CGACAAGGCATTTCGCTACCTTAAGAGGGTTATAGTTACCCCCGCCGTTT |
| 361 | 23S: A12 | AACTGAACTCCAGCTTCACGTGCCAGCACTGGGCAGGTGTCGCCCTCTGT |
| 362 | 23S: A13 | CTAGCAGAGAGCTATGTTTTTATTAAACAGTCGGGCCCCCCTAGTCACTG |
| 363 | 23S: A14 | TTAAAACGCCTTAGCCTACTCAGCTAGGGGCACCTGTGACGGATCTCGGT |
| 364 | 23S: A15 | ACAAAACTAACTCCCTTTTCAAGGACTCCATGAATCAGTTAAACCAGTAC |
| 365 | 23S: A16 | ATAATGCCTACACCTGGTTCTCGCTATTACACCTCTCCCCAGGCTTAAAC |
| 366 | 23S: A17 | CAATCCTACAAAACATATCTCGAAGTGTCAGAAATTAGCCCTCAACGTCA |
| 367 | 23S: A18 | CTTTGCTGCTACTACTACCAGGATCCACATACCTGCAAGGTCCAAAGGAA |
| 368 | 23S: A19 | CAACCCACACAGGTCGCCACTCTACACAATCACCAAAAAAAAGGTGTTCC |
| 369 | 23S: A20 | GGATTAATTCCCGTCCATTTTAGGTGCCTCTGACCTCGATGGGTGATCTG |
| 370 | 23S: A21 | AGGGTGGCTGCTTCTAAGCCCACCTTCCCATTGTCTTGGGCCAAAGACTC |
| 371 | 23S: A22 | GTATTTAGGGGCCTTAACCATAGTCTGAGTTGTTTCTCTTTCGGGACACA |
| 372 | 23S: A23 | CCTCACTCCAACCTTCTACGACGGTGACGAGTTCGGAGTTTTACAGTACG |
| 373 | 23S: A24 | CCCTAAACGTCCAATTAGTGCTCTACCCCGCCACCAACCTCCAGTCAGGC |
| 374 | 23S: A25 | AATAGATCGACCGGCTTCGGGTTTCAATGCTGTGATTCCAGGCCCTATTA |
| 375 | 23S: A26 | ACAACGCTGCGGGCATATCGGTTTCCCTACGACTACAAGGATAAAAACCT |
| 376 | 23S: A27 | ACAAAGAACTCCCTGGCCCGTGTTTCAAGACGGACGATGCAACACTAGTC |
| 377 | 23S: A28 | ACAATGTTACCACTGATTCTTTCGGAAGAATTCATTCCTTACGCGCCACA |
| 378 | 23S: A29 | CTGGTTTCAGGTACTTTTCACCCCCCTATAGGGGTACTTTTCAGCATTCC |
| 379 | 23S: A30 | CTCTATCGGTCTTGAGACGTATTTAGAATTGGAAGTTGATGCCTCCCACA |
| 380 | 23S: A31 | ATCACCCTCTACGGTTCTAAAATTCCAAATAAAATTCGATTTATCCCACG |
| 381 | 23S: A32 | TCTATACACCACATCTCCCTAATATTACTAAAAGGGATTCAGTTTGTTCT |
| 382 | 23S: A33 | GCCGTTACTAACGACATCGCATATTGCTTTCTTTTCCTCCGCCTACTAAG |
| 383 | 23S: A34 | GGGTTCCCAATCCTACACGGATCAACACAAAAAAAATGTGCTAGGAAGTC |
| 384 | 5S: A1 | ACTACTGGGATCGAAACGAGACCAGGTATAACCCCCATGCTATGACCGCA |
| 385 | MM_16S_P10 | GCGTATGCCTGGAGAATTGGAATTCTTGTTACTCATACTAACAGTGTTGC |
| 386 | MM_16S_P11 | GATTAACCCAATTTTAAGTTTAGGAAGTTGGTGTAAATTATGGAATTAAT |
| 387 | MM_16S_P12 | AGCTTGAACGCTTTCTTTATTGGTGGCTGCTTTTAGGCCTACAATGGTTA |
| 388 | MM_16S_P13 | ATTATTCACTATTAAAGGTTTTTTCCGTTCCAGAAGAGCTGTCCCTCTTT |
| 389 | MM_16S_P14 | CTTACTTTTTGATTTTGTTGTTTTTTTAGCAAGTTTAAAATTGAACTTAA |
| 390 | MM_16S_P15 | AACCAGCTATCACCAAGCTCGTTAGGCTTTTCACCTCTACCTAAAAATCT |
| 391 | MM_16S_P7 | AATACTTGTAATGCTAGAGGTGATGTTTTTGGTAAACAGGCGGGGTTCTT |
| 392 | MM_16S_P8 | TTTATCTTTTTGGATCTTTCCTTTAGGCATTCCGGTGTTGGGTTAACAGA |
| 393 | MM_16S_P9 | TTATTTATAGTGTGATTATTGCCTATAGTCTGATTAACTAACAATGGTTA |
| 394 | RN_16S_P4 | AGTGATTGTAGTTGTTTATTCACTATTTAAGGTTTTTTCCTTTTCCTAAA |
| 395 | RN_16S_P5 | TGGCTATATTTTAAGTTTACATTTTGATTTGTTGTTCTGATGGTAAGCTT |
| 396 | RN_16S_P6 | TTTTTTTAATCTTTCCTTAAAGCACGCCTGTGTTGGGCTAACGAGTTAGG |
| 397 | RN_16S_P7 | TGTTGGGTTAGTACCTATGATTCGATAATTGACAATGGTTATCCGGGTTG |
| 398 | RN_16S_P8 | AGGAGAATTGGTTCTTGTTACTCATATTAACAGTATTTCATCTATGGATC |
| 399 | RN_16S_P9 | TTTGTGATATAGGAATTTATTGAGGTTTGTGGAATTAGTGTGTGTAAGTA |
| 400 | MM_28S_P1 | GCCGGGGAGTGGGTCTTCCGTACGCCACATTTCCCACGCCGCGACGCGCG |
| 401 | MM_28S_P10 | ACCTCGGGCCCCCGGGCGGGGCCCTTCACCTTCATTGCGCCACGGCGGCT |
| 402 | MM_28S_P14 | TCGCGTCCAGAGTCGCCGCCGCCGCCGGCCCCCCGAGTGTCCGGGCCCCC |
| 403 | MM_28S_P15 | CGCTGGTTCCTCCCGCTCCGGAACCCCCGCGGGGTTGGACCCGCCGCCCC |
| 404 | MM_28S_P16 | CGCCGACCCCCGACCCGCCCCCCGACGGGAAGAAGGAGGGGGGAAGAGAG |
| 405 | MM_28S_P17 | GGGACGACGGGGCCCCGCGGGGAAGAGGGGAGGGCGGGCCCGGGCGGAAA |
| 406 | MM_28S_P18 | GGCGCCGCGCGGAAAACCGCGGCCCGGGGGGCGGACCCGGCGGGGGAACA |
| 407 | MM_28S_P19 | CCCCCACACGCGCGGGACACGCCCGCCCGCCCCCGCCACGCACCTCGGGA |
| 408 | MM_28S_P2 | CACCCGCTTTGGGCTGCATTCCCAAGCAACCCGACTCCGGGAAGACCCGA |
| 409 | MM_28S_P20 | TGGAGCGAGGCCCCGCGGGGAGGGGACCCGCGCCGGCACCCGCCGGGCTC |
| 410 | MM_28S_P21 | CGAGGCCGGCGTGCCCCGACCCCGACGCGAGGACGGGGCCGGGCGCCGGG |
| 411 | MM_28S_P22 | TCCCCGGAGCGGGTCGCGCCCGCCCGCACGCGCGGGACGGACGCTTGGCG |
| 412 | MM_28S_P23 | TCCACACGAACGTGCGTTCAACGTGACGGGCGAGAGGGCGGCCCCCTTTC |
| 413 | MM_28S_P24 | TCCCAAGACGAACGGCTCTCCGCACCGGACCCCGGTCCCGACGCCCGGCG |
| 414 | MM_28S_P25 | CCGCCGCGGGGACGACGCGGGGACCCCGCCGAGCGGGGACGGACGGGGAC |
| 415 | MM_28S_P3 | GCACCGCCACGGTGGAAGTGCGCCCGGCGGCGGCCGGTCGCCGGCCGGGG |
| 416 | MM_28S_P6 | CCCACCGGGCCCCGAGAGAGGCGACGGAGGGGGGTGGGAGAGCGGTCGCG |
| 417 | MM_28S_P7 | CCCGGCCCCCACCCCCACGCCCGCCCGGGAGGCGGACGGGGGGAGAGGGA |
| 418 | MM_28S_P8 | TATCTGGCTTCCTCGGCCCCGGGATTCGGCGAAAGCGCGGCCGGAGGGCT |
| 419 | MM_28S_P9 | CGCCGCCGACCCCGTGCGCTCGGCTTCGTCGGGAGACGCGTGACCGACGG |
| 420 | RN_28S_P12 | GCGCCCCCCCGCACCCGCCCCGTCCCCCCCGCGGACGGGGAAGAAGGGAG |
| 421 | RN_28S_P14 | CGAACCCCGGGAACCCCCGACCCCGCGGAGGGGGAAGGGGGAGGACGAGG |
| 422 | RN_28S_P16 | CACCCGGGGGGGCGACGAGGCGGGGACCCGCCGGACGGGGACGGACGGGG |
| 423 | RN_28S_P17 | GCCAACCGAGGCTCCTTCGGCGCTGCCGTATCGTTCCGCTTGGGCGGATT |
| 424 | RN_28S_P4 | CCCGGGCCCCCGGACCCCCGAGAGGGACGACGGAGGCGACGGGGGGTGGG |
| 425 | RN_28S_P5 | TGGGAGGGGCGGCCCGGCCCCCGCGACCGCCCCCCTTTCCGCCACCCCAC |
| 426 | RN_28S_P6 | GGGAGAGGCCGGGGGGAGAGCGCGGCGACGGGTATCCGGCTCCCTCGGCC |
| 427 | RN_28S_P7 | CGCTGCTGCCGGGGGGCTGTAACACTCGGGGCGGGGTGGTCCGGCGCCCA |
| 428 | RN_28S_P8 | CGCCGCCGACCCCGTGCGCTCGGCTTCGCTCCCCCCCACCCCGAGAAGGG |

In one embodiment, the RNA sample is from a human and the DNA probe set includes probes specific to human unwanted RNA species such as rRNA and mitochondrial mRNA transcripts as described in this disclosure. In another embodiment, a DNA probe set for depleting unwanted RNA from a human RNA sample includes probes specific to human rRNA and mitochondrial mRNA transcripts, and probes specific to Gram positive and Gram negative unwanted RNA transcripts as described in this disclosure. In a further embodiment, a DNA probe set for depleting unwanted RNA from a human RNA sample includes probes specific to an Archaea bacterial species, an example of which is M. smithii as described in this disclosure. As such, in some embodiments, a DNA probe set for depleting rRNA from a human RNA sample comprises only probes directed to human unwanted RNA species or comprises a mixed DNA probe set that targets non-human unwanted RNA transcripts as well. A skilled artisan will understand that the probe set to be used for RNA depletion will depend on the research intentions for the sample, the environment from which the sample was taken, and any other factors that lead into an experimental design for RNA depletion of an RNA sample.

In one embodiment, the RNA sample is from a non-human eukaryote and the DNA probe set includes probes specific to unwanted RNA in that eukaryotic sourced sample. For example, if the RNA sample is from a mouse or rat, the DNA probe set would include probes specific to mouse or rat unwanted RNA species, which may also include DNA probes specific to unwanted Gram positive and Gram negative bacterial RNA species as well, or other bacterial species such as Archaea species.

In some embodiments, the DNA probes do not hybridize to the entire contiguous length of an RNA species to be deleted. Surprisingly, it was found during experimentation that the full length sequence of a RNA species targeted for depletion need not be targeted with a full-length DNA probe, or a probe set that tiles contiguously over the entire RNA sequence; indeed the DNA probes described herein leave gaps such that the DNA:RNA hybrids formed are not contiguous. Surprisingly, gaps of at least 5 nt, 10 nt, 15 nt or 20 nt between DNA:RNA hybrids provided efficient RNA depletion. Further, probe sets that include gaps can hybridize more efficiently to the unwanted RNA, as the DNA probes do not hinder hybridization of adjacent probes as could potentially occur with probes that cover the whole RNA sequence targeted for depletion, or probes that overlap one another.

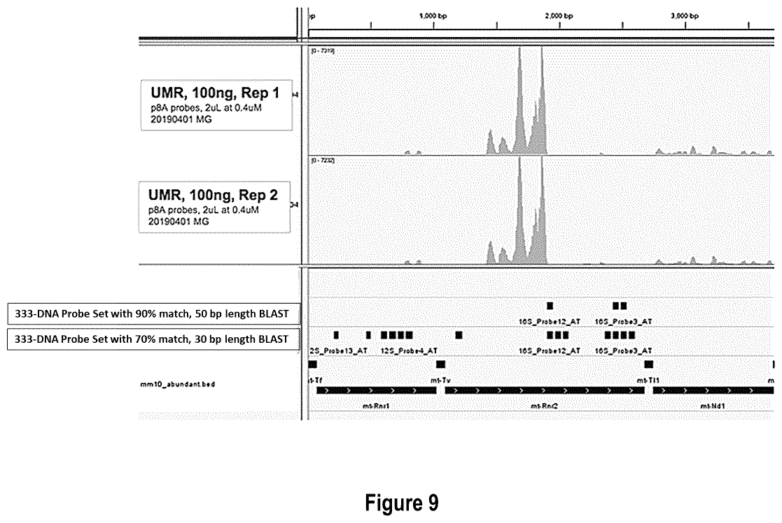

In addition, probe sets can be supplemented to improve RNA depletion methods for a given species. A method of supplementing a probe set for use in depleting off-target RNA nucleic acid molecules from a nucleic acid sample can comprise: a) contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid; b) contacting the DNA:RNA hybrids with a ribonuclease that degrades the RNA from the DNA:RNA hybrids, thereby degrading the off-target RNA molecules in the nucleic acid sample to form a degraded mixture; c) separating the degraded RNA from the sample; d) sequencing the remaining RNA from the sample; e) evaluating the remaining RNA sequences for the presence of off-target RNA molecules from the first species, thereby determining gap sequence regions; and f) supplementing the probe set with at least one DNA probe complementary to discontiguous sequences in one or more of the gap sequence regions. In some embodiments, the gap sequence regions comprise at least 50, or at least 60, or at least 70 base pairs. In some embodiments, the first species is a non-human species and the second species is human. In some embodiments, the first species is rat or mouse. Exemplary methods for supplementing a probe set for improved depletion of off-target rRNA nucleic acid molecules in mouse samples are outlined in Example 8 and FIG. 9.

In some embodiments, a first species is a non-human species and a second species is human. In some embodiments, a first species is rat or mouse. In some embodiments, the second species is human, Gram-positive bacteria, Gram-negative bacteria, or a mixture thereof.

### Compositions and Kits

In one embodiment, the present disclosure relates to compositions comprising a probe set as described herein. In some embodiments, the composition comprises the probe set and a ribonuclease capable of degrading RNA in a DNA:RNA hybrid, such as RNase H or Hybridase. In some embodiments, the probe set comprises at least two DNA probes complementary to at least one off-target rRNA molecule in the nucleic acid sample, wherein the probes are non-overlapping and are discontiguous relative to the length of the off-target rRNA molecule (e.g., at least 5 or at least 10 bases apart along the full length). In some embodiments, the composition comprises the probe set comprising at least two DNA probes hybridized to at least one off-target RNA molecule, wherein each DNA probe is hybridized at least 5, or at least 10, bases apart along the length of the off-target RNA molecule from any other DNA probe in the probe set. In some embodiments, the composition comprises a nucleic acid destabilizing chemical such as formamide, betaine, DMSO, glycerol, or derivatives or mixtures thereof. In one embodiment, the destabilizing chemical is formamide or a derivative thereof which is present in a concentration of between 10-45% of the hybridization total reaction volume.

In one embodiment, the present disclosure describes a kit comprising a probe set comprising at least two DNA probes complementary to discontiguous sequences along the full length of at least one off-target rRNA molecule (e.g., at least 5 bases apart or at least 10 bases apart along the full length) in a nucleic acid sample, a ribonuclease capable of degrading RNA in a DNA:RNA hybrid. In some embodiments, the probe set comprises any of the DNA probes described herein, or any combination thereof.

In some embodiments, a kit comprises a buffer and nucleic acid purification medium. In some embodiments, the kit comprises one or more of a buffer, a nucleic acid purification medium, and a DNA probe set as described herein. In some embodiments, the probe set comprises two or more sequences of SEQ ID NOs: 1-333. In some embodiments, the probe set comprises two or more sequences of SEQ ID NOs: 1-428. In some embodiments, a probe set comprises two or more, or five or more, or 10 or more, or 25 or more, or 50 or more, or 100 or more, or 150 or more, or 200 or more, or 250 or more, or 300 or more, or 350 or more, or 377 sequences from SEQ ID NOs: 1-377 (human, Gram-positive bacteria, Gram-negative bacteria, and Archaea). In some embodiments, a probe set comprises two or more, or five or more, or 10 or more, or 25 or more, or 50 or more, or 100 or more, or 150 or more, or 200 or more, or 250 or more, or 300 or more, or 350 or more, or 384 sequences from SEQ ID NOs: 1-333 and SEQ ID NOs: 378-428 (human, Gram-positive bacteria, Gram-negative bacteria, mouse, and rat). In some embodiments, a probe set comprises two or more, or five or more, or 10 or more, or 25 or more, or 44 sequences from SEQ ID NOs: 334-377 (Archaea). In some embodiments, a probe set comprises two or more, or five or more, or 10 or more, or 25 or more, or 50 or more, or 51 sequences from SEQ ID NOs: 378-428 (mouse and rat).

In some embodiments, the kit comprises: 1) probe set as described herein; 2) a ribonuclease; 3) a DNase; and 4) RNA purification beads. In some embodiments, the kit comprises an RNA depletion buffer, a probe depletion buffer, and a probe removal buffer.

### Analysis of Depleted Samples

The disclosed methods also find utility in analyzing transcriptomes from single or mixed samples. Transcriptomic analysis can be impeded by high relative abundance of ribosomal RNA, for example a sample may comprise >85% of rRNA molecules in total RNA from bacterial cells. With such high amounts of rRNA competing for sequencing or other analysis reagents it can be difficult to focus on the more informative parts of a transcriptome which can get lost in the background of unwanted rRNA analysis. The disclosed methods can facilitate rich transcriptome analysis of microbial or eukaryotic isolates, for example, at low inputs of DNA, leading to lower rRNA sequencing reads, enabling lower sequencing costs and enabling metatranscriptomic analysis of low biomass samples. This is exemplified in Example 4, where low input amounts (<80 ng) from mixed samples were evaluated using the RNase H rRNA depletion methods described in this disclosure. The methods described herein can be used in conjunction with a variety of downstream applications, such as creating libraries for nucleic acid sequencing techniques, using the enriched samples in RT-PCR followed by microarray analysis, PCR, qPCR, etc. However, it should be understood that the enriched RNA samples resulting from the RNA depletion methods described here are not limited to any particular downstream application, such as sequencing.

As an example, the RNA depleted samples can be used to create sequencing libraries, such that the libraries created can be attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid can be an automated process. Preferred embodiments include sequencing-by-synthesis (“SBS”) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. SBS can utilize nucleotide monomers that have a terminator moiety or those that lack any terminator moieties. Methods utilizing nucleotide monomers lacking terminators include, for example, pyrosequencing and sequencing using 7-phosphate-labeled nucleotides. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

Sequencing methodologies that can leverage the RNA depletion workflows and RNA enriched samples include, but are not limited to, cycle sequencing that is accomplished by stepwise addition of reversible terminator nucleotides containing, for example, a cleavable or photobleachable dye. Examples of Illumina instruments that can leverage the methods described herein include HiSeq™, MiSeq™, NextSeq™, NovaSeq™, NextSeq™, and iSeq™ commercial instruments.

Additional sequencing techniques include sequencing by ligation. Such techniques utilize DNA ligase to incorporate oligonucleotides and identify the incorporation of such oligonucleotides.

Further, nanopore sequencing can also use the disclosed RNA depleted samples for library preparation. Nanopore sequencing methods sequence a strand of nucleic acids that pass through a pore wherein change is current through the pore is characteristic of which nucleotide is passing through the pore.

Further, sequencing using real-time monitoring of DNA polymerase activity can utilize the RNA depleted samples.

Additional SBS technologies that can create libraries for sequencing using the RNA depleted samples described herein include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary).

Additional downstream application that can leverage the enriched samples following RNA depletion as described herein include PCR, qPCR, microarray analysis, etc. For example, microarray analysis is a powerful technique for studying gene expression. The enriched samples can be used in microarray analysis by converting the enriched RNA to cDNA following methods known to a skilled artisan (e.g., reverse-transcriptase polymerase chain reaction RT-PCR). The cDNA could then be immobilized on substrates, microarray probes applied and expression analysis determined following any number of microarray analysis methodologies (for example, Agilent, Affymetrix, and Illumina to name a few sell commercial microarray analysis systems). Polymerase chain reaction (PCR) or quantitative PCR (qPCR) could also utilize the enriched sample as a substrate following established techniques (Current Protocols for Molecular Biology).

As such, the RNA depleted samples resulting from the methods described herein can be used to create sequencing libraries, amplification products, and the like which can be utilized for downstream analysis methodologies. The disclosed methods are not limited by any downstream application.

### EXAMPLES

The following examples are illustrative only and are not intended to limit the scope of the application. Modifications will be apparent and understood by skilled artisans and are included within the spirit and under the disclosure of this application.

### Example 1—Depletion of Unwanted RNA Species from a Sample

In this example total RNA is the target nucleic acid in the sample, and RNA depletion involves four main steps: 1) hybridization, 2) RNase H treatment, 3) DNase treatment, and 4) target RNA clean up.

Hybridization is accomplished by annealing a defined DNA probe set to denatured RNA in a sample. A RNA sample, 10-100 ng, is incubated in a tube with 1 μL of a 1 μM/oligo DNA oligo probe set (probes corresponding to SEQ ID NOs: 1-333, as listed in Table 1), 3 μL of 5× Hybridization buffer (500 mM Tris HCl pH 7.5 and 1000 mM KCl), 2.5 μL of 100% formamide and enough water for a total reaction volume of 15 μL. The hybridization reaction is incubated at 95° C. for 2 min to denature the nucleic acids, slow cooled to 37° C. by decreasing temperature 0.1° C./sec and held at 37° C. No incubation time needed once the reaction reaches 37° C. The total time it takes for denaturation to reach 37° C. is about 15 min.

Following hybridization, the following components are added to the reaction tube for RNase H removal of the unwanted RNA species from the DNA:RNA duplex; 4 μL 5× RNase H buffer (100 mM Tris pH 7.5, 5 mM DTT, 4 0 mM MgCl₂) and 1 μL RNase H enzyme. The enzymatic reaction is incubated at 37° C. for 30 min. The reaction tube can be held on ice.

Following the removal of the RNA from the DNA:RNA hybrid, the DNA probes are degraded. To the 20 μL reaction tube, the following components are added: 3 μL 10× Turbo DNase buffer (200 mM Tris pH 7.5, 50 mM CaCl₂), 20 mM MgCl₂), 1.5 μL Turbo DNase (Thermo Fisher Scientific) and 5.5 μL H₂O for a total volume of 30 μL. The enzymatic reaction is incubated at 37° C. for 30 min followed by 75° C. for 15 min. The 75° C. incubation can serve to fragment the target total RNA to desired insert sizes for use in downstream processing, in this example the target insert size is around 200 nt of total RNA. The timing of this incubation step can be adjusted depending on the insert size needed for subsequent reactions, as known to a skilled artisan. Following incubation, the reaction tube can be held on ice.

After hybridization of the probes to the unwanted RNA, removal of the RNA, and removal of the DNA, the target total RNA in the sample can be isolated from the reaction conditions. The reaction tube is taken from 4° C. and allowed to come to room temperature and 60 μL of RNAClean XP beads (Beckman Coulter) are added and the reaction tube is incubated for 5 min. Following incubation, the tube is placed on a magnet for 5 min., after which the supernatant is gently removed and discarded. While still on the magnet, the beads with the attached total RNA are washed twice in 175 μL fresh 80% EtOH. After the second wash, the beads are spun down in a microcentrifuge to pellet the beads at the bottom of the tube, the tube is placed back on the magnet and the EtOH is removed, being careful to remove as much of the residual EtOH as possible without disturbing the beads. The beads are air dried for a few minutes, resuspended in 9.5 μL of ELB buffer (Illumina), allowed to sit a few more minutes at RT and placed back on the magnet to collect the beads. 8.5 μL of the supernatant is transferred to a fresh tube and placed on ice for additional downstream processing, such as created cDNA from the target total RNA.

In another example, 100 ng total RNA is diluted in 11 μL nuclease-free ultrapure water in each well of a 96-well PCR plate. To each well is added 4 μL of DNA probes (SEQ ID NOs: 1-333) in hybridization buffer and the well contents are mixed and optionally centrifuged. The plate is heated at 95° C. for 2 min and then the temperature is reduced at 0.1° C. per second until the temperature reaches 37° C. and then held at 37° C. to hybridize the probes. The plate is centrifuged at 280×g for 10 seconds. To degrade the DNA:RNA hybrids, to each well is added 5 μL of RNase in buffer and the well contents are mixed. The plate is heated at 37° C. for 15 min and then held at 4° C. To each well is added 10 μL of DNase in buffer and the well contents are mixed. The plate is heated at 37° C. for 15 min and then held at 4° C. The sample plate is centrifuged at 280×g for 10 seconds. To each well is added 60 μL RNAClean XP beads and the well contents are mixed. The plate is incubated at room temperature for 5 min. The plate is placed on a magnetic stand until the supernatant is clear (about 5 min). The supernatant in each well is removed and discarded. The beads are washed twice with 80% ethanol. Residual ethanol is removed from each well and the plate is air-dried on the magnetic stand for 1 min. To each well is added 10.5 μL of elution buffer, the well contents are mixed, and the plate is incubated at room temperature for 2 min. The plate is sealed and centrifuged at 280×g for 10 seconds. The plate is placed on a magnetic stand until the supernatant is clear (about 2 min). From each well, 8.5 μL of supernatant is transferred to the corresponding well of a new plate.

### Example 2—cDNA Synthesis

Further processing of the RNA from Example 1 could be making a library preparation from the RNA target nucleic acids that can be sequenced for example by NGS. To 8.5 μL of the final reaction from Example 1, 8.5 μL of Elute, Prime High Concentration Random Hexamer Mix buffer (EPH buffer, TruSeq Stranded Total RNA Kit, Illumina) is added for a total volume of 17 μL. The sample is incubated at 65° C. for 2 min to denature the nucleic acids. Following denaturation, the reaction tube can be held on ice. First strand synthesis is performed by adding 8 μL of a reverse transcription enzyme mix (9 μL First Strand Synthesis Mix (FSA, TruSeq Stranded Total RNA Kit, Illumina) and 1 μL Protoscript II RT, (NEB)) to the denatured sample for a total volume of 25 μL. The reaction mix is incubated in a heated lid thermocycler under the following conditions: 25° C. for 5 min, 42° C. for 25 min, 70° C. for 15 min. Once the first strand synthesis reaction is complete the reaction tube can be held on ice.

Second strand cDNA synthesis can be performed by adding 5 μL Resuspension Buffer (RSB, TruSeq Stranded Total RNA Kit, Illumina) and 20 μL Second Strand Marking Mix (SSM buffer, TruSeq Stranded Total RNA Kit, Illumina) to the iced sample. The reaction tube is incubated at 16° C. for 60 min, and the sample may then be held on ice.

Following the cDNA synthesis steps, the cDNA can be cleaned up and separated from reaction components by, for example, adding 90 μL of SPB (Illumina) to the reaction tube and incubating for 5 min at RT. Following incubation, the tube is placed on a magnet for around 8 min to collect the paramagnetic beads and the supernatant is gently removed and discarded. While still on the magnet, the beads are washed twice with 175 μL fresh 80% EtOH. Following the washes, the beads are centrifuged to the bottom of the tube, the tube is place back on the magnet and EtOH is gently removed and discarded. The beads are dried for a few minutes and resuspended in 18.5 μL RSB, mixed well and allowed to incubate at RT for around 5 min before placed back on the magnet. Depending on the downstream application, the desired amount of purified cDNA can be removed to a new tube. In this example, a library prep for downstream sequencing is being made so 17.5 μL of the supernatant is transferred to a new tube which can be kept on ice.

### Example 3—Library Preparation for Next Generation Sequencing

One method for preparing a library for sequencing includes A-tailing cDNA fragments, ligating adaptors, amplifying target fragments, and quantifying resultant fragments prior to sequencing.

The tube with 17.5 μL of purified cDNA from Example 2 is used for processing. To the purified cDNA is added 12.5 μL ATL (Illumina) for A-tailing the fragments. The reaction tube is incubated at 37° C. for 30 min followed by incubating at 70° C. for 5 min and the tube is put back on ice. Adaptors are ligated to the A-tailed sample by added in order: 2.5 μL RSB, 2.5 μL Index Adaptors (TruSeq Stranded Total RNA Kit, Illumina) and 2.5 μL of Ligation buffer (Illumina). The reaction tube is incubated at 30° C. for 10 min after which point 5 μL of Stop Ligation buffer (Illumina) is added and the reaction is held on ice.

Once the adaptor ligation reaction is completed, the ligated fragments are separated from the reaction components. To purify the adaptor ligated fragments, 34 μL SPB is added to the reaction tube which is incubated at RT for around 5 min. The tube is then placed on a magnet for capturing the paramagnetic beads and the beads are washed twice with 175 μL 80% EtOH, the EtOH being gently removed after the second wash. Following a 3 min air dry of the beads, the beads are resuspended in 52 μL RSB, the slurry in mixed, allowed to sit at RT for an additional 5 min, and placed back on the magnet. The supernatant (50 μL) is transferred to a fresh tube for a second round of bead cleanup.

For the second round, 40 μL SPB is added to the 50 μL sample and the process described above is repeated except the final purified fragments are resuspended in 21 μL of RSB and 20 μL of the final purified sample is transferred to a new reaction tube for subsequent amplification which increases the amount of target sequence for optimized sequencing results.

To the 20 μL of purified adaptor ligated sample, 5 μL of PCR primer cocktail (PPC, TruSeq Stranded Total RNA Kit, Illumina) and 25 μL PPM (TruSeq Stranded Total RNA Kit, Illumina) are added and the following amplification program in a heated lid thermocycler is performed: 98° C. for 30 sec followed by the cycled program 98° C. at 10 sec, 60° C. at 30 sec, 72° C. at 30 sec. The number of amplification cycles is dependent on the amount of RNA input at the beginning of the whole process. For example, for 100 ng RNA, approximately 12-13 cycles can be adequate, for 10 ng 15-16 cycles, and for 1 ng 17-18 cycles may be needed. The number of amplification cycles is typically optimized for any preparation as known to a skilled artisan.

The amplicons can be purified away from reaction conditions by adding 50 μL SPB to the reaction tube, incubate at RT, centrifuge the tube to pellet the beads and magnetically capture the beads. The supernatant can be discarded and the beads washed as previously stated followed by resuspension of the washed beads in 26 μL RSB, magnetic bead capture and transfer of the supernatant containing the DNA library for sequencing to a fresh tube. The library is typically quantified and analyzed prior to sequencing, for example by measuring an aliquot using the Qubit™ High Sensitivity kit (Thermo Fisher Scientific) and/or running an aliquot on a Bioanalyzer (Agilent). A skilled artisan will appreciate the many ways in which nucleic acids in a sample can be quantitated.

The resulting library preparation can then be used for next generation sequencing, microarray analysis or other downstream applications. For applications such as sequencing, the library preparation methodology is determined by the sequencing instrument being used and the companion library preparation method defined for that sequencing instrument. In this example, the library preparation method is characteristic of library creation when sequencing on Illumina sequencing instruments. A skilled artisan will understand that library preparation methods may vary depending in sequencing instrumentation, as such the present examples are exemplary only and the present RNA depletion methods are not limited to any particular library preparation workflow. Indeed, the present methods provide a RNA depleted sample that can input into any downstream applications that would benefit from a RNA sample depleted of unwanted RNA species.

### Example 4—Microbial Transcriptome Analysis

In this example, microbial isolates, a mixed sample of bacterial species, and a standard cell mix were obtained from ATCC for testing.

| Sample type | Microbial species tested |
|---|---|
| Microbial | E. coli, B. subtilis, S. Epidermidis, E. cloacae and |
| isolates | B. cereus |
| ATCC- | A. baumannii, A. odontolyhticus, B. cereus, B. vulgatus, |
| MSA2002 | B. adolescentis, C. beijerinckii, C. acnes, D. radiodurans, |
| 20 strain | E. faecalis, E. coli, H. pylori, L. gasseri., N. meningitidis, |
| mix | P. gingivalis, P. aeruginosa, R. sphaeroides, S. aureus, |
| | S. epidermidis, S. agalactiae, S. mutans |
| ATCC | B. tragilis, B. vulgatus, B. adolescentis, C. difficile, |
| MSA2006 | E. faecalis, L. plantarum, E. cloacae, E. coli, H. pylori, |
| Human gut | S. enterica, y. enterococolitica, F. nucleatum |
| mix | |

Total RNA can be extracted using the RNeasy Power Microbiome Kit (Qiagen) following manufacturer's protocol and evaluated for integrity and quantified by Bioanalyzer RNA Electrophoresis (Agilent). 10-250 ng of total RNA from each sample can be used for rRNA depletion following either the RiboZero methodology (Illumina, following manufacturer's protocol) or the methods disclosed herein using RNase H enzymatic degradation of unwanted rRNA. Ribo-depleted and non-ribo depleted RNA (control) samples can be prepared for sequencing using the TruSeq Stranded Total RNA Sample prep kit (Illumina) following manufacturer's instructions. Libraries can be pooled and sequenced, for example, on a MiSeq or NextSeq sequencing instrument (Illumina) for 2×76 paired end reads.

Sequence filtering, alignment, and transcript coverage can be performed using the online BaseSpace Sequencing Hub (BSSH) and the following exemplary workflows, for example: 1) Partition rRNA Sequences App (parse rRNA sequences to denote as abundant sequences in analysis), 2) RNA Custom Genome Builder App (create STAR-compatible microbial transcriptome), and 3) RNA-Seq Alignment App (STAR-alignment and salmon-transcript quantification). To quantify rRNA from multiple strains within the microbial samples rRNA sequences can be retrieved from NCBI annotated genomes and used as inputs to the BSSH workflow.

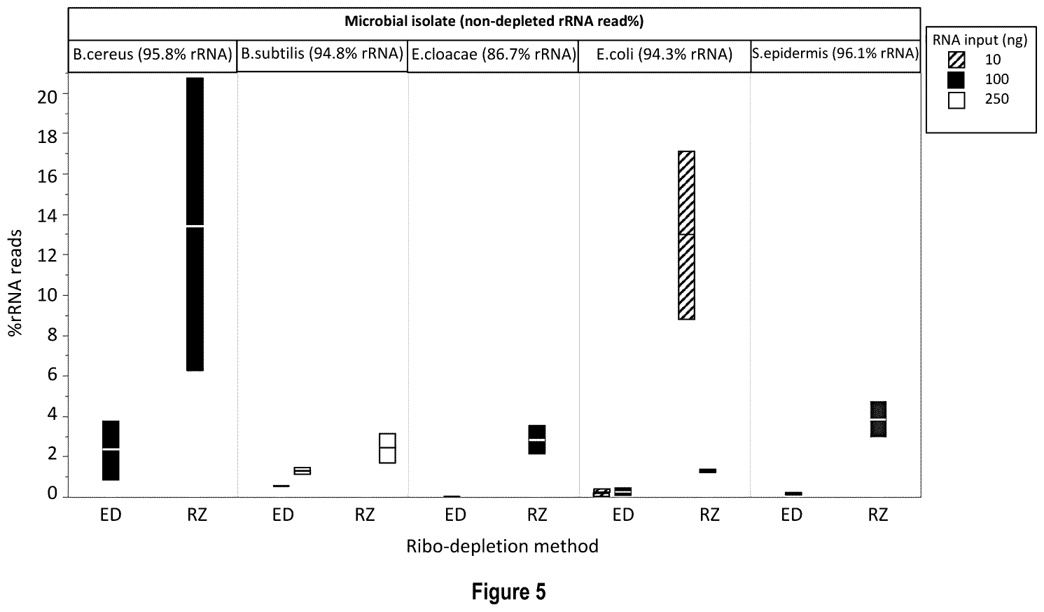

The transcriptomes of the microbial isolates, microbial mixtures and control samples were sequenced and % rRNA reads compared. The RNase H enzymatic method disclosed herein is highly effective in depleting unwanted rRNA in the tested species (<5% rRNA reads). Ribosomal RNA depletion is most significant for the E. coli low input sample (10 ng) using the RNase H method comparative to the established RiboZero method; <0.5% vs 13% average rRNA reads, respectively (FIG. 5).

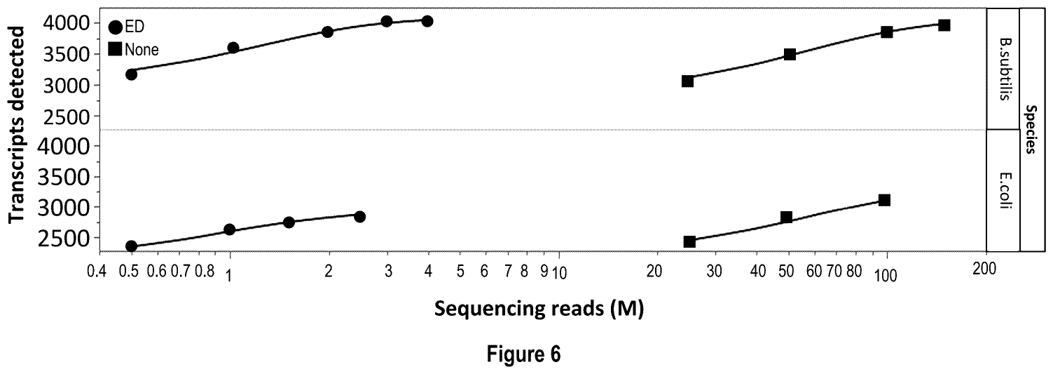

Data was used to access the enrichment of biologically important RNA reads when the RNase H rRNA depletion method was used and compared to no rRNA depletion. FIG. 6 demonstrates the results of an assessment where, in general, a 20-50× reduction in read depth was seen for a B. subtilis or E. coli sample if the sample was rRNA depleted prior to library preparation and sequencing using the RNase H methods compared to no rRNA depletion.

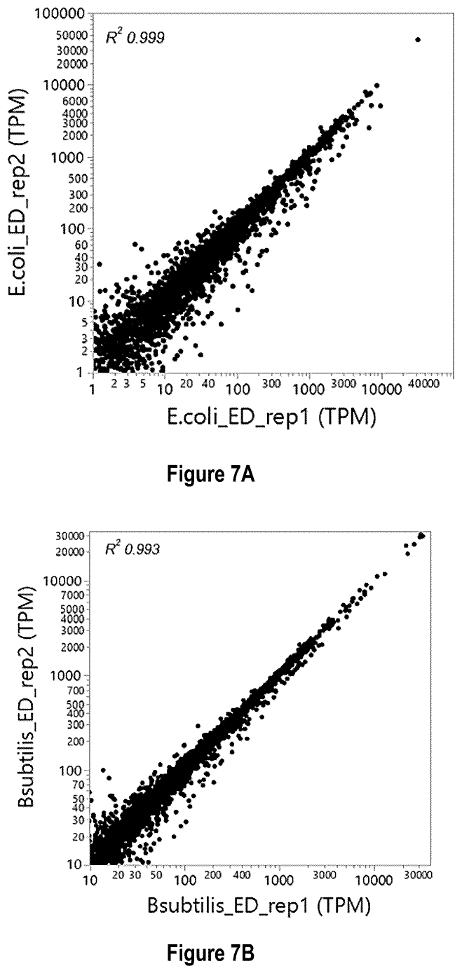

Data collected was evaluated to determine the reproducibility of the experimental microbial transcriptome sequencing efforts. Pairwise linear regression of gene expression levels was determined between the RNase H rRNA depleted replicates for E. coli and B. subtilis as example systems. High correlation (R²>0.99) indicated the ability of the RNase H rRNA depletion method to reproducibly remove rRNA from samples (FIG. 7).

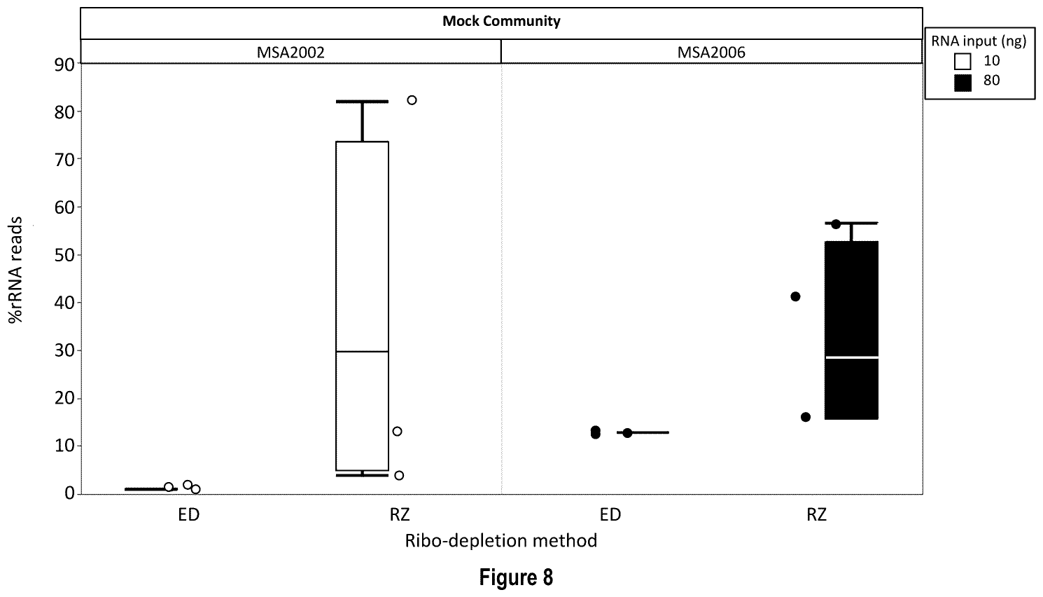

For evaluating whether the RNase H enzymatic rRNA depletion method might be useful for rRNA depletion of mixed samples, FIG. 8 demonstrates exemplary data for the mixed samples of 20 strain MSA2002 and human gut MSA2006 in triplicate. Low input samples of 10 mg total RNA from MSA2002 or 80 ng total RNA from MSA2006 was used for rRNA depletion methods. For the 20 strain MSA2002 samples, the RNase H rRNA depletion method reduced rRNA reads by 83% or <2% of sequence reads while the RiboZero method of rRNA depletion resulted in a more variable and higher rRNA abundance compared to non-depleted samples. For the 12 strain MSA2006 samples, the same outcome was seen where RNase H method reduced rRNA reads by approximately 95% to <13% of the sequencing reads comparative to non-depleted samples, the RiboZero method yielded more variable results.

As such, it was determined that in experiments for evaluating samples, either mixed or otherwise, the RNase H rRNA depletion method provides a robust and effective workflow for reducing unwanted rRNA in samples for high quality microbial whole transcriptome research. The RNase H rRNA depletion method was also very effective and compatible with low input samples.

### Example 5—Effect of Formamide on RNA Depletion

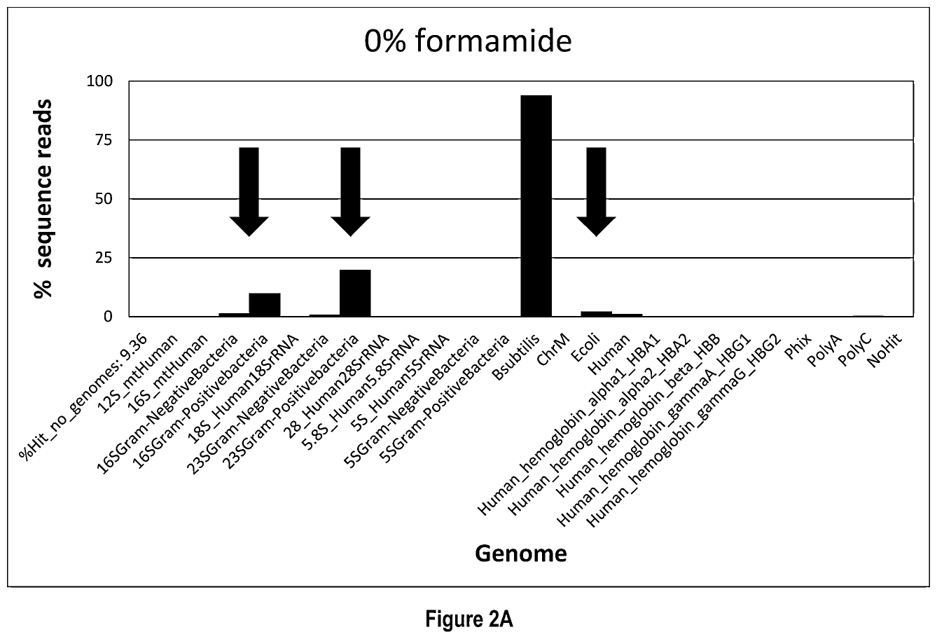

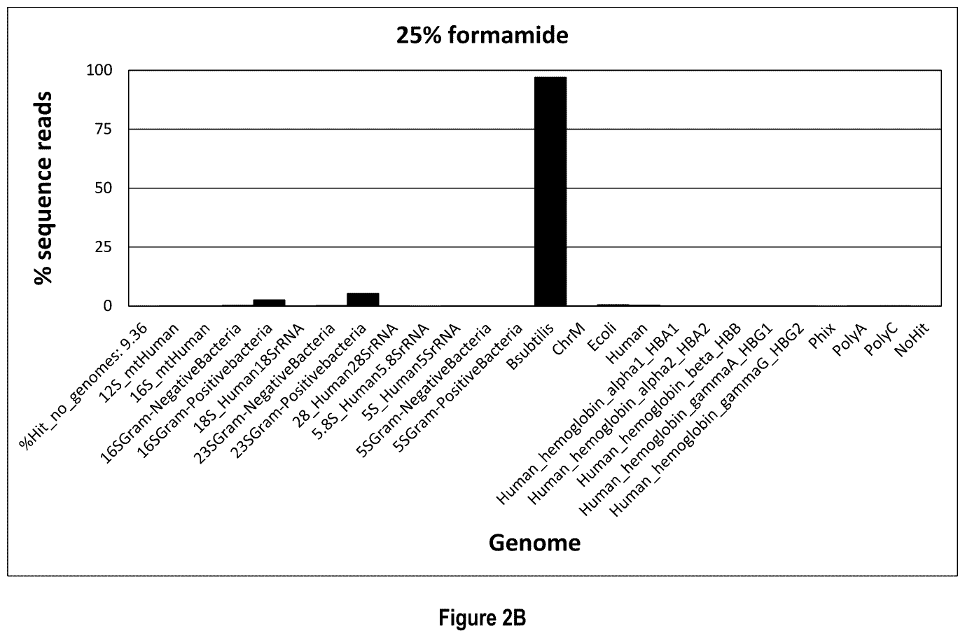

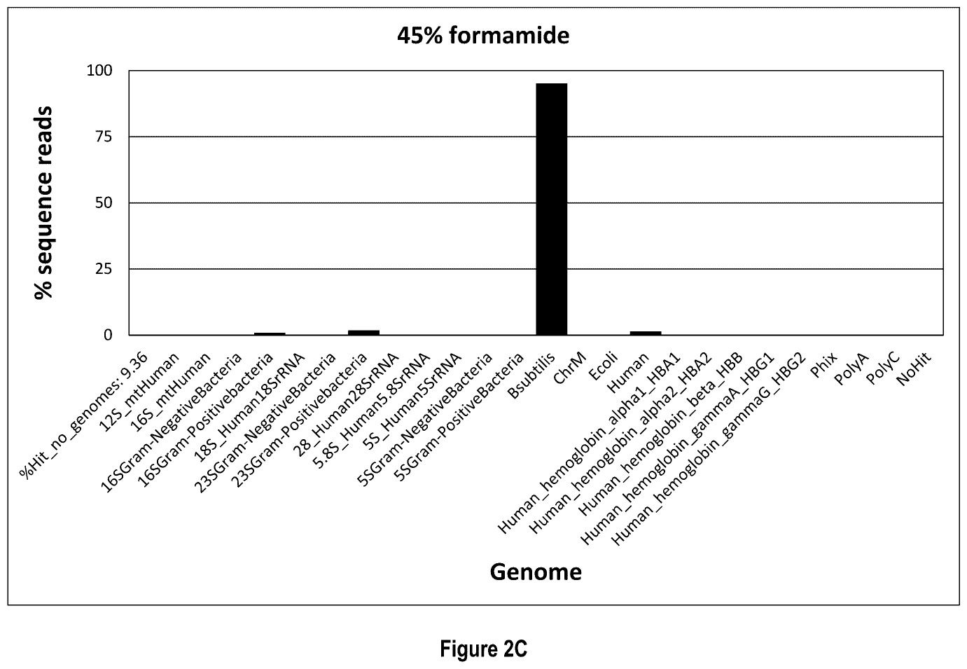

FIG. 2 shows exemplary data where an RNA sample has been depleted of unwanted RNA species. The RNA sample was depleted of unwanted RNA using the methods described herein, while evaluating the effects of formamide concentration on unwanted RNA depletion. In this example, the DNA probes targeted depletion of unwanted rRNA species from Gram positive bacteria (23S, 16S, 5S), Gram negative bacteria (23S, 16S, 5S including), human mitochondria (16S, 12S), human rRNAs (28S, 18S, 5.8S, 5S), human hemoglobin mRNAs (HBA-A1, HBA-A2, HBB, HBG1, HBG2) while the target RNA species is total RNA from B. subtilis. As the concentration of formamide increases the percentage of unwanted RNA species reads significantly decreases. For example, no formamide during RNA depletion resulted in off target RNA reads for Gram positive 23S and 16S and Gram negative (including E. coli) bacteria 23S and 16S, including E. coli specific sequences. The addition of 25% formamide to the hybridization reaction resulted in undetectable off target reads for Gram negative 23S and 16S (with significant reduction in off targets reads specific to E. coli) and significantly reduced off target reads for the Gram positive 23S and 16S. The addition of formamide to 45% of the hybridization reaction saw additional significant decreases in off target reads for the Gram positive undesired rRNA 23S and 16S as well as a further drop in off target E. coli reads. As such, the addition of formamide to the RNA depletion hybridization reaction is shown to increase the amount of Gram positive and Gram negative undesired RNAs depleted as evidenced by the reduction in off target reads for those species. In general, it was found that the addition of formamide improves depletion of the unwanted rRNA transcripts. When using B. subtilis RNA as the target RNA for analysis, for example, assaying for E. coli and human rRNA sequences can provide a measure of potential contamination.

### Example 6—Variation of Input Starting Material

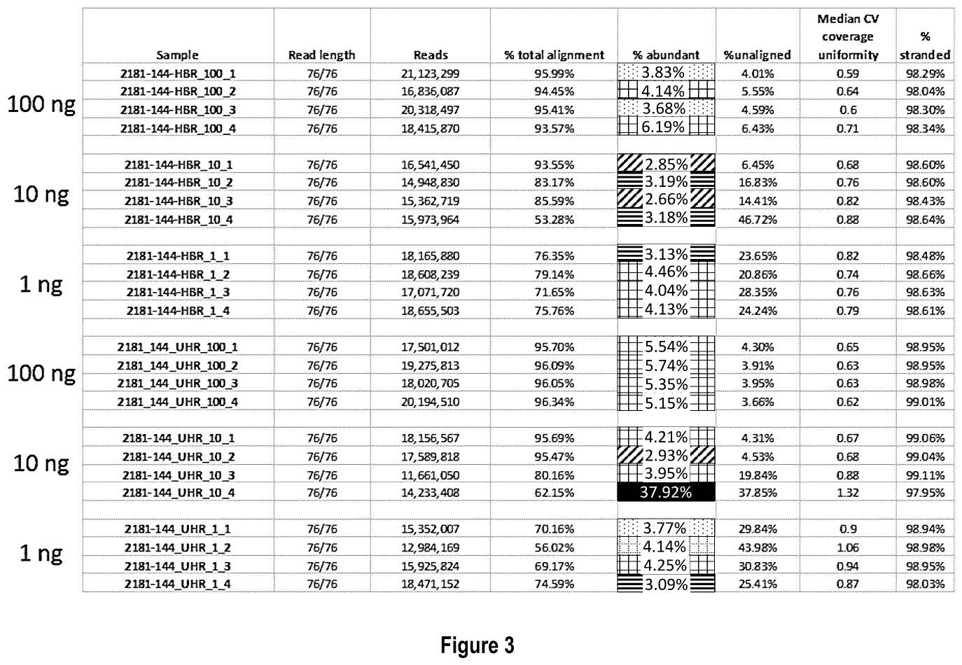

Experiments were performed to identify the impact of input starting material on RNA depletion and subsequent downstream analysis, such as shown in FIG. 3 where RNA depleted and enriched RNA samples from human brain (HBR) and a universal human RNA (UHR) were used to create libraries for sequencing on the Illumina NextSeq™ 500 or 550 sequencing instrument. Following RNA depletion using 100 ng, 10 ng or 1 ng of input samples, sequencing libraries were prepared as exemplified in Examples 1-3. Sequencing was performed as recommended by the NextSeq™ user guide following by data analysis using two BaseSpace (Illumina) applications, RNASeq Alignment application and the RNAExpress application. Data analysis for B. subtilis and E. coli presence was also performed using a modified tool Fastqscreen (https://www.bioinformatics.babraham.ac.uk/projects/fastq_screen/). The data shows that the RNA depletion remains constant for both HBR and UHR regardless of amount of input of the RNA sample and the % total alignment for the target RNA, while decreasing with decreasing input amounts, still shows that actionable and useful sequence data can be gathered even when using 1 ng of input sample. Further, in a comparative experiment the current method for RNA depletion leads to fewer % abundance of non-target reads at all input levels (100 ng˜3%, 25 ng˜4%, 10 ng˜3% and 1 ng˜3%) when compared to data when using RiboZero rRNA depletion kit (Epicentre) for RNA depletion (100 ng˜3%; 25 ng˜5%, 10 ng˜8% and 1 ng˜35%) or NEBNext rRNA depletion methods (NEB) (100 ng˜8%, 25 ng˜8%, 10 ng˜9% and 1 ng˜30%).

### Example 7—RNA Depletion of Mouse and Rat RNA Samples

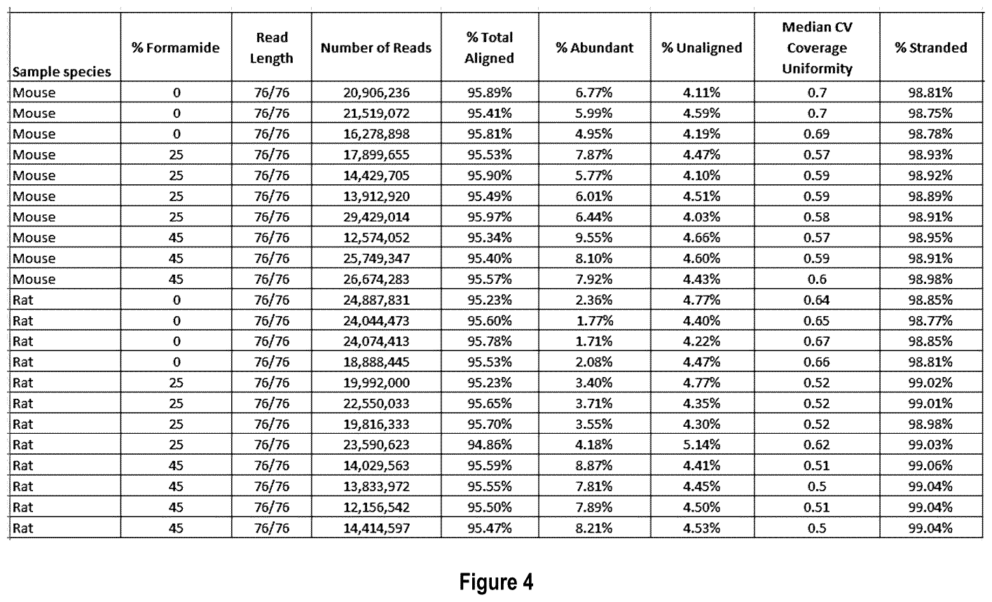

To demonstrate that the RNA depletion methods can be useful for non-human RNA samples both mouse and rat RNA samples were used for RNA depletion methods. For FIG. 4, either mouse or rat RNA samples were depleted of unwanted RNA using equivalent methods and DNA probes as for human RNA samples. Formamide was again varied for each rodent species, including no formamide, 25% formamide or 45% formamide in the hybridization reaction. While total % aligned reads is not affected with the increase in formamide, there may be a trend toward an increase in detection of non-target reads as formamide increases. As such, the addition of formamide to the hybridization reaction maybe useful in some sample types, as it can improve detection of some transcripts so its addition should be optimized.

### Example 8—Preparation of Supplemental Mouse Probes

Within the pool of 333 DNA probes described above for enzymatic removal of unwanted sequences (SEQ ID NOs: 1-333), the DNA oligonucleotides for eukaryotic rRNA depletion were designed based upon the major human rRNA transcripts, namely 5S, 5.8S, 18S, and 28S, as well as the two mitochondrial rRNA sequences, 12S and 16S. When tested on human total RNA, this 333-DNA probe pool was very effective at removing rRNA reads. However, when tested with mouse (Mus musculus) or rat (Rattus norvegicus) total RNA samples, depletion was less robust, suggesting that the probes did not hybridize and remove some regions of rodent rRNA sequences efficiently because these mouse and rat regions were divergent from human sequences.

The fastq files containing the total sequencing reads obtained from the 333-DNA probe experiment were aligned to mouse and rat ribosomal RNA sequences and to the 333 DNA probe sequences. The alignment results showed that probe coverage across all the ribosomal RNA sequences was generally good, but there were some regions where probe sequences did not align as well to rodent rRNAs. More specifically, the majority of the mouse and rat rRNA reads that did not align to the probe pool map belonged to either the 28S or 16S rodent rRNA transcripts (Table 2). The alignments were done with Bowtie2 (See Langmead and Salzberg, Nature Methods 2012, 9:357-359), version 2.1.0 with its default settings. Most of the ribosomal RNA that did not get depleted with the 333 DNA probe enzymatic method were from the same regions that lacked probe alignment (FIG. 9).

TABLE 2
Mouse/Rat Genbank sequences used for the study

| Genome | 16S | 28S |
|---|---|---|
| Mus musculus | NC_005089.1: 1094-2675 | NR_003279.1 |
| Rattus norvegicus | NC_001665.2: 1094-2664 | NR_046246.1 |

To deplete these regions more effectively, additional probes were designed to cover the regions identified above for mouse and rat ribosomal RNA sequences. To minimize the number of additional probes and probe redundancies, additional probes were designed against the gaps in mouse rRNA sequences, then these data were informatically pooled together with the 333 DNA probe set to identify any remaining gaps in rat rRNA coverage by aligning the combined pool to rat rRNA transcripts. This sequential process yielded a total of 44 additional oligonucleotide probes, to provide a supplemental pool of 377 probes. Sequencing experiments as described above were repeated with the 377 DNA probe set. In both mouse and rat samples, addition of the 44 new probes resulted in a decrease in the percentage of rRNA reads from the libraries compared to the 333-DNA probe set, showing increased depletion efficiency (Table 3).

TABLE 3
Percent ribosomal RNA in sequencing
reads with 333- and 377-Probe Sets

| RNase H Probe Set | Mouse Sample | Rat Sample |
|---|---|---|
| 333 DNA Probe Set | 9.5% | 5.3% |
| 377 DNA Probe Set | 7.0% | 3.7% |

Supplementation of the 333 DNA probe pool with additional probes against certain rodent sequences improved rRNA depletion in the tested rodent samples. Exemplary probes against mouse 16S include SEQ ID NOs: 385 to 393. Exemplary probes against mouse 28S include SEQ ID NOs: 400 to 419. Exemplary probes against rat 16S include SEQ ID NOs: 394 to 399. Exemplary probes against rat 28S include SEQ ID NOs: 420 to 428.

1. A method for depleting off-target RNA molecules from a nucleic acid sample comprising:
a. contacting a nucleic acid sample comprising at least one target RNA or DNA sequence and at least one off-target RNA molecule with a probe set comprising at least two DNA probes complementary to discontiguous sequences along the full length of the at least one off-target RNA molecule, thereby hybridizing the DNA probes to the off-target RNA molecules to form DNA:RNA hybrids, wherein each DNA:RNA hybrid is at least 5 bases apart, or at least 10 bases apart, along a given off-target RNA molecule sequence from any other DNA:RNA hybrid; and
b. contacting the DNA:RNA hybrids with a ribonuclease that degrades the RNA from the DNA:RNA hybrids, thereby degrading the off-target RNA molecules in the nucleic acid sample to form a degraded mixture, wherein the ribonuclease is hybridase.

2. The method of claim 1, further comprising:
a. degrading any remaining DNA probes by contacting the degraded mixture with a DNase I to form a DNA degraded mixture; and
b. separating the degraded RNA from the degraded mixture or the DNA degraded mixture.

3. The method of claim 1, wherein the contacting with the probe set comprises treating the nucleic acid sample with formamide wherein the formamide is present during the contacting with the probe set at a concentration of from about 10 to 45% by volume.

4. The method of claim 1, wherein the contacting with the probe set comprises applying heat above the melting temperature of the at least one DNA:RNA hybrid.

5. The method of claim 1, wherein the nucleic acid sample is from a human or from a non-human eukaryote, bacterium, virus, plant, soil, or a mixture thereof, optionally wherein the non-human eukaryote is a rat, mouse, or non-human primate.

6. The method of claim 1, wherein the off-target RNA is rRNA and/or globin mRNA.

7. The method of claim 1, wherein the probe set comprises at least two DNA probes that hybridize to at least one off-target RNA molecule selected from:
a. 28S, 18S, 5.8S, 5S, 16S, and/or 12S from humans;
b. HBA-A1, HBA-A2, HBB, HBG1, and HBG2 from hemoglobin, and 23S, 16S, and 5S from Gram positive or Gram negative bacteria; and/or
c. rat 16S, rat 28S, mouse 16S, and mouse 28S, and combinations thereof.

8. The method of claim 1, wherein the probe set comprises DNA probes to a particular off-target RNA molecule that are complementary to about 80 to 85% of the sequence of the off-target RNA molecule,
optionally wherein the DNA probes comprise:
a. two or more sequences selected from SEQ ID NOs: 1-333; or
b. two or more sequences selected from SEQ ID NOs: 1-428; or
c. two or more sequences selected from SEQ ID NOs: 1-377; or
d. two or more sequences selected from SEQ ID NOs: 1-333 and SEQ ID NOs: 378-428; or
e. two or more sequences selected from SEQ ID NOs: 334-377; or
f. two or more sequences selected from SEQ ID NOs: 378-428;
or a combination thereof.

9. A composition comprising
a. a probe set comprising at least two DNA probes complementary to discontiguous sequences along the full length of at least one off-target RNA molecule in a nucleic acid sample; and
b. a ribonuclease capable of degrading RNA in a DNA:RNA hybrid; wherein the ribonuclease is hybridase and/or each DNA probe is hybridized at least 5, or at least 10, bases apart along the full length of the at least one off-target RNA molecule from any other DNA probe in the probe set.

10. The composition of claim 9, further comprising DNase I for degrading any remaining DNA probes.

11. The composition of claim 9, further comprising formamide wherein the formamide is present at about 10 to 45% by volume when the DNA probes come in contact with the nucleic acid sample.

12. The composition of claim 9, wherein the off-target RNA is rRNA and/or globin mRNA.

13. The composition of claim 9, wherein the probe set comprises at least two DNA probes that hybridize to at least one off-target RNA molecule selected from:
a. 28S, 18S, 5.8S, 5S, 16S, and/or 12S from humans;
b. HBA-A1, HBA-A2, HBB, HBG1, and HBG2 from hemoglobin, and 23S, 16S, and 5S from Gram positive or Gram negative bacteria; and/or
c. rat 16S, rat 28S, mouse 16S, and mouse 28S, and combinations thereof.

14. The composition of claim 9, wherein the probe set comprises DNA probes comprising:
a. two or more sequences selected from SEQ ID NOs: 1-333; or
b. two or more sequences selected from SEQ ID NOs: 1-428; or
c. two or more sequences selected from SEQ ID NOs: 1-377; or
d. two or more sequences selected from SEQ ID NOs: 1-333 and SEQ ID NOs: 378-428; or
e. two or more sequences selected from SEQ ID NOs: 334-377; or
f. two or more sequences selected from SEQ ID NOs: 378-428;
g. or a combination thereof.

15. A kit comprising:
a. a probe set comprising at least two DNA probes complementary to discontiguous sequences at least 5, or at least 10, bases apart along the full length of at least one off-target RNA molecule in a nucleic acid sample;
b. a ribonuclease capable of degrading RNA in a DNA:RNA hybrid, wherein the ribonuclease is hybridase; and
c. DNase I capable of degrading any remaining DNA probes; and
d. formamide wherein the formamide is present during the contacting with the probe set at a concentration of from about 10 to 45% by volume.

16. The kit of claim 15, wherein the off-target RNA is rRNA and/or globin mRNA.

17. The kit of claim 15, wherein the probe set comprises at least two DNA probes that hybridize to at least one off-target RNA molecule selected from:
a. 28S, 18S, 5.8S, 5S, 16S, and/or 12S from humans;
b. HBA-A1, HBA-A2, HBB, HBG1, and HBG2 from hemoglobin, and 23S, 16S, and 5S from Gram positive or Gram negative bacteria; and/or
c. rat 16S, rat 28S, mouse 16S, and mouse 28S, and combinations thereof.

18. The kit of claim 15, wherein the probe set comprises DNA probes to a particular off-target RNA molecule that are complementary to about 80 to 85% of the sequence of the off-target RNA molecule,
optionally wherein the DNA probes comprise:
a. two or more-sequences selected from SEQ ID NOs: 1-333; or
b. two or more sequences selected from SEQ ID NOs: 1-428; or
c. two or more sequences selected from SEQ ID NOs: 1-377; or
d. two or more sequences selected from SEQ ID NOs: 1-333 and SEQ ID NOs: 378-428; or
e. two or more sequences selected from SEQ ID NOs: 334-377; or
f. two or more sequences selected from SEQ ID NOs: 378-428;
or a combination thereof.

19. The kit of claim 15, further comprising:
a. a probe set comprising SEQ ID NOs: 1-333; and
b. RNA purification beads.

20. A method of supplementing a probe set for use in depleting off-target RNA nucleic acid molecules from a nucleic acid sample comprising:
a. contacting a nucleic acid sample comprising at least one RNA or DNA target sequence and at least one off-target RNA molecule from a first species with a probe set comprising at least two DNA probes complementary to discontiguous sequences along the full length of the at least one off-target RNA molecule from a second species, thereby hybridizing the at least two DNA probes to the off-target RNA molecules to form DNA:RNA hybrids, wherein
i. each DNA:RNA hybrid is at least 5 bases apart, or at least 10 bases apart, along a given off-target RNA molecule sequence from any other DNA:RNA hybrid;
b. contacting the DNA:RNA hybrids with hybridase that degrades the RNA from the DNA:RNA hybrids, thereby degrading the off-target RNA molecules in the nucleic acid sample to form a degraded mixture;
c. separating the degraded RNA from the degraded mixture;
d. sequencing the remaining RNA from the sample;
e. evaluating the remaining RNA sequences for the presence of off-target RNA molecules from the first species, thereby determining gap sequence regions; and
f. supplementing the probe set with additional DNA probes complementary to discontiguous sequences in one or more of the gap sequence regions.

21. The method of claim 20, wherein the composition of claim 9 is used to supply the hybridase and the probe set comprising the DNA probes complementary to discontiguous sequences along the full length of the at least one off-target RNA molecule of a human.

22. The method of claim 20, wherein the method is used to identify DNA probes that hybridize to one or more off-target RNA molecules from rat and/or mouse, optionally selected from rat 16S, rat 28S, mouse 16S, and mouse 28S, and combinations thereof.


SEQUENCE LISTING (428 sequences)

SEQ ID NO: 1 - DNA, 50 bp, synthetic construct
  misc_feature 1..50: note = DNA Probe
  source 1..50: mol_type = other DNA
gttcgtccaagtgcactttccagtacacttaccatgttacgacttgtctc

SEQ ID NO: 2 - DNA, 50 bp, synthetic construct
  misc_feature 1..50: note = DNA Probe
  source 1..50: mol_type = other DNA
taggggttttagttaaatgtcctttgaagtatacttgaggagggtgacgg

SEQ ID NO: 3 - DNA, 50 bp, synthetic construct
  misc_feature 1..50: note = DNA Probe
  source 1..50: mol_type = other DNA
ttcagggccctgttcaactaagcactctactctcagtttactgctaaatc

SEQ ID NO: 4 - DNA, 50 bp, synthetic construct
  misc_feature 1..50: note = DNA Probe
  source 1..50: mol_type = other DNA
agtttcataagggctatcgtagttttctggggtagaaaatgtagcccatt

SEQ ID NO: 5 - DNA, 50 bp, synthetic construct
  misc_feature 1..50: note = DNA Probe
  source 1..50: mol_type = other DNA
ggctacaccttgacctaacgtctttacgtgggtacttgcgcttactttgt

SEQ ID NO: 6 - DNA, 50 bp, synthetic construct
  misc_feature 1..50: note = DNA Probe
  source 1..50: mol_type = other DNA
ttgctgaagatggcggtatataggctgagcaagaggtggtgaggttgatc

SEQ ID NO: 7 - DNA, 50 bp, synthetic construct
  misc_feature 1..50: note = DNA Probe
  source 1..50: mol_type = other DNA
cagaacaggctcctctagagggatatgaagcaccgccaggtcctttgagt

SEQ ID NO: 8 - DNA, 50 bp, synthetic construct
  misc_feature 1..50: note = DNA Probe
  source 1..50: mol_type = other DNA
gtagtgttctggcgagcagttttgttgatttaactgttgaggtttagggc

SEQ ID NO: 9 - DNA, 50 bp, synthetic construct
  misc_feature 1..50: note = DNA Probe
  source 1..50: mol_type = other DNA
atctaatcccagtttgggtcttagctattgtgtgttcagatatgttaaag

SEQ ID NO: 10 - DNA, 50 bp, synthetic construct
  misc_feature 1..50: note = DNA Probe
  source 1..50: mol_type = other DNA
attttgtgtcaactggagttttttacaactcaggtgagttttagctttat

SEQ ID NO: 11 - DNA, 50 bp, synthetic construct
  misc_feature 1..50: note = DNA Probe
  source 1..50: mol_type = other DNA
ctaaaacactctttacgccggcttctattgacttgggttaatcgtgtgac

SEQ ID NO: 12 - DNA, 50 bp, synthetic construct
  misc_feature 1..50: note = DNA Probe
  source 1..50: mol_type = other DNA
gaaattgaccaaccctggggttagtatagcttagttaaactttcgtttat

SEQ ID NO: 13 - DNA, 50 bp, synthetic construct
  misc_feature 1..50: note = DNA Probe
  source 1..50: mol_type = other DNA
actgctgtttcccgtgggggtgtggctaggctaagcgttttgagctgcat

SEQ ID NO: 14 - DNA, 50 bp, synthetic construct
  misc_feature 1..50: note = DNA Probe
  source 1..50: mol_type = other DNA
gcttgtcccttttgatcgtggtgatttagagggtgaactcactggaacgg

SEQ ID NO: 15 - DNA, 50 bp, synthetic construct
  misc_feature 1..50: note = DNA Probe
  source 1..50: mol_type = other DNA
taatcttactaagagctaatagaaaggctaggaccaaacctatttgttta

SEQ ID NO: 16 - DNA, 50 bp, synthetic construct
  misc_feature 1..50: note = DNA Probe
  source 1..50: mol_type = other DNA
aaaccctgttcttgggtgggtgtgggtataatactaagttgagatgatat

SEQ ID NO: 17 - DNA, 50 bp, synthetic construct
  misc_feature 1..50: note = DNA Probe
  source 1..50: mol_type = other DNA
gcgctttgtgaagtaggccttatttctcttgtcctttcgtacagggagga

SEQ ID NO: 18 - DNA, 50 bp, synthetic construct
  misc_feature 1..50: note = DNA Probe
  source 1..50: mol_type = other DNA
aaaccgacctggattactccggtctgaactcagatcacgtaggactttaa

SEQ ID NO: 19 - DNA, 50 bp, synthetic construct
  misc_feature 1..50: note = DNA Probe
  source 1..50: mol_type = other DNA
acctttaatagcggctgcaccatcgggatgtcctgatccaacatcgaggt

SEQ ID NO: 20 - DNA, 50 bp, synthetic construct
  misc_feature 1..50: note = DNA Probe
  source 1..50: mol_type = other DNA
tgatatggactctagaataggattgcgctgttatccctagggtaacttgt

SEQ ID NO: 21 - DNA, 50 bp, synthetic construct
  misc_feature 1..50: note = DNA Probe
  source 1..50: mol_type = other DNA
attggatcaattgagtatagtagttcgctttgactggtgaagtcttagca

SEQ ID NO: 22 - DNA, 50 bp, synthetic construct
  misc_feature 1..50: note = DNA Probe
  source 1..50: mol_type = other DNA
ttgggttctgctccgaggtcgccccaaccgaaatttttaatgcaggtttg

SEQ ID NO: 23 - DNA, 50 bp, synthetic construct
  misc_feature 1..50: note = DNA Probe
  source 1..50: mol_type = other DNA
tgggtttgttaggtactgtttgcattaataaattaaagctccatagggtc

SEQ ID NO: 24 - DNA, 50 bp, synthetic construct
  misc_feature 1..50: note = DNA Probe
  source 1..50: mol_type = other DNA
gtcatgcccgcctcttcacgggcaggtcaatttcactggttaaaagtaag

SEQ ID NO: 25 - DNA, 50 bp, synthetic construct
  misc_feature 1..50: note = DNA Probe
  source 1..50: mol_type = other DNA
cgtggagccattcatacaggtccctatttaaggaacaagtgattatgcta

SEQ ID NO: 26 - DNA, 50 bp, synthetic construct
  misc_feature 1..50: note = DNA Probe
  source 1..50: mol_type = other DNA
ggtaccgcggccgttaaacatgtgtcactgggcaggcggtgcctctaata

SEQ ID NO: 27 - DNA, 50 bp, synthetic construct
  misc_feature 1..50: note = DNA Probe
  source 1..50: mol_type = other DNA
gtgatgtttttggtaaacaggcggggtaaggtttgccgagttccttttac

SEQ ID NO: 28 - DNA, 50 bp, synthetic construct
  misc_feature 1..50: note = DNA Probe
  source 1..50: mol_type = other DNA
cttatgagcatgcctgtgttgggttgacagtgagggtaataatgacttgt

SEQ ID NO: 29 - DNA, 50 bp, synthetic construct
  misc_feature 1..50: note = DNA Probe
  source 1..50: mol_type = other DNA
attgggctgttaattgtcagttcagtgttttgatctgacgcaggcttatg

SEQ ID NO: 30 - DNA, 50 bp, synthetic construct
  misc_feature 1..50: note = DNA Probe
  source 1..50: mol_type = other DNA
tcatgttacttatactaacattagttcttctatagggtgatagattggtc

SEQ ID NO: 31 - DNA, 50 bp, synthetic construct
  misc_feature 1..50: note = DNA Probe
  source 1..50: mol_type = other DNA
agttcagttatatgtttgggattttttaggtagtgggtgttgagcttgaa

SEQ ID NO: 32 - DNA, 50 bp, synthetic construct
  misc_feature 1..50: note = DNA Probe
  source 1..50: mol_type = other DNA
tggctgcttttaggcctactatgggtgttaaattttttactctctctaca

SEQ ID NO: 33 - DNA, 50 bp, synthetic construct
  misc_feature 1..50: note = DNA Probe
  source 1..50: mol_type = other DNA
gtccaaagagctgttcctctttggactaacagttaaatttacaaggggat

SEQ ID NO: 34 - DNA, 50 bp, synthetic construct
  misc_feature 1..50: note = DNA Probe
  source 1..50: mol_type = other DNA
ggcaaatttaaagttgaactaagattctatcttggacaaccagctatcac

SEQ ID NO: 35 - DNA, 50 bp, synthetic construct
  misc_feature 1..50: note = DNA Probe
  source 1..50: mol_type = other DNA
tgtcgcctctacctataaatcttcccactattttgctacatagacgggtg

SEQ ID NO: 36 - DNA, 50 bp, synthetic construct
  misc_feature 1..50: note = DNA Probe
  source 1..50: mol_type = other DNA
tcttaggtagctcgtctggtttcgggggtcttagctttggctctccttgc

SEQ ID NO: 37 - DNA, 50 bp, synthetic construct
  misc_feature 1..50: note = DNA Probe
  source 1..50: mol_type = other DNA
taattcattatgcagaaggtataggggttagtccttgctatattatgctt

SEQ ID NO: 38 - DNA, 50 bp, synthetic construct
  misc_feature 1..50: note = DNA Probe
  source 1..50: mol_type = other DNA
tctttcccttgcggtactatatctattgcgccaggtttcaatttctatcg

SEQ ID NO: 39 - DNA, 50 bp, synthetic construct
  misc_feature 1..50: note = DNA Probe
  source 1..50: mol_type = other DNA
ggtaaatggtttggctaaggttgtctggtagtaaggtggagtgggtttgg

SEQ ID NO: 40 - DNA, 50 bp, synthetic construct
  misc_feature 1..50: note = DNA Probe
  source 1..50: mol_type = other DNA
taatgatccttccgcaggttcacctacggaaaccttgttacgacttttac

SEQ ID NO: 41 - DNA, 50 bp, synthetic construct
  misc_feature 1..50: note = DNA Probe
  source 1..50: mol_type = other DNA
aagttcgaccgtcttctcagcgctccgccagggccgtgggccgaccccgg

SEQ ID NO: 42 - DNA, 50 bp, synthetic construct
  misc_feature 1..50: note = DNA Probe
  source 1..50: mol_type = other DNA
ggcctcactaaaccatccaatcggtagtagcgacgggcggtgtgtacaaa

SEQ ID NO: 43 - DNA, 50 bp, synthetic construct
  misc_feature 1..50: note = DNA Probe
  source 1..50: mol_type = other DNA
caacgcaagcttatgacccgcacttactcgggaattccctcgttcatggg

SEQ ID NO: 44 - DNA, 50 bp, synthetic construct
  misc_feature 1..50: note = DNA Probe
  source 1..50: mol_type = other DNA
ccgatccccatcacgaatggggttcaacgggttacccgcgcctgccggcg

SEQ ID NO: 45 - DNA, 50 bp, synthetic construct
  misc_feature 1..50: note = DNA Probe
  source 1..50: mol_type = other DNA
ctgagccagtcagtgtagcgcgcgtgcagccccggacatctaagggcatc

SEQ ID NO: 46 - DNA, 50 bp, synthetic construct
  misc_feature 1..50: note = DNA Probe
  source 1..50: mol_type = other DNA
ctcaatctcgggtggctgaacgccacttgtccctctaagaagttggggga

SEQ ID NO: 47 - DNA, 50 bp, synthetic construct
  misc_feature 1..50: note = DNA Probe
  source 1..50: mol_type = other DNA
ggtcgcgtaactagttagcatgccagagtctcgttcgttatcggaattaa

SEQ ID NO: 48 - DNA, 50 bp, synthetic construct
  misc_feature 1..50: note = DNA Probe
  source 1..50: mol_type = other DNA
caccaactaagaacggccatgcaccaccacccacggaatcgagaaagagc

SEQ ID NO: 49 - DNA, 50 bp, synthetic construct
  misc_feature 1..50: note = DNA Probe
  source 1..50: mol_type = other DNA
cctgtccgtgtccgggccgggtgaggtttcccgtgttgagtcaaattaag

SEQ ID NO: 50 - DNA, 50 bp, synthetic construct
  misc_feature 1..50: note = DNA Probe
  source 1..50: mol_type = other DNA
ctggtggtgcccttccgtcaattcctttaagtttcagctttgcaaccata

SEQ ID NO: 51 - DNA, 50 bp, synthetic construct
  misc_feature 1..50: note = DNA Probe
  source 1..50: mol_type = other DNA
aaagactttggtttcccggaagctgcccggcgggtcatgggaataacgcc

SEQ ID NO: 52 - DNA, 50 bp, synthetic construct
  misc_feature 1..50: note = DNA Probe
  source 1..50: mol_type = other DNA
ggcatcgtttatggtcggaactacgacggtatctgatcgtcttcgaacct

SEQ ID NO: 53 - DNA, 50 bp, synthetic construct
  misc_feature 1..50: note = DNA Probe
  source 1..50: mol_type = other DNA
gattaatgaaaacattcttggcaaatgctttcgctctggtccgtcttgcg

SEQ ID NO: 54 - DNA, 50 bp, synthetic construct
  misc_feature 1..50: note = DNA Probe
  source 1..50: mol_type = other DNA
cacctctagcggcgcaatacgaatgcccccggccgtccctcttaatcatg

SEQ ID NO: 55 - DNA, 50 bp, synthetic construct
  misc_feature 1..50: note = DNA Probe
  source 1..50: mol_type = other DNA
accaacaaaatagaaccgcggtcctattccattattcctagctgcggtat

SEQ ID NO: 56 - DNA, 50 bp, synthetic construct
  misc_feature 1..50: note = DNA Probe
  source 1..50: mol_type = other DNA
ctgctttgaacactctaattttttcaaagtaaacgcttcgggccccgcgg

SEQ ID NO: 57 - DNA, 50 bp, synthetic construct
  misc_feature 1..50: note = DNA Probe
  source 1..50: mol_type = other DNA
gcatcgagggggcgccgagaggcaaggggcggggacgggcggtggctcgc

SEQ ID NO: 58 - DNA, 50 bp, synthetic construct
  misc_feature 1..50: note = DNA Probe
  source 1..50: mol_type = other DNA
ccgcccgctcccaagatccaactacgagctttttaactgcagcaacttta

SEQ ID NO: 59 - DNA, 50 bp, synthetic construct
  misc_feature 1..50: note = DNA Probe
  source 1..50: mol_type = other DNA
gctggaattaccgcggctgctggcaccagacttgccctccaatggatcct

SEQ ID NO: 60 - DNA, 50 bp, synthetic construct
  misc_feature 1..50: note = DNA Probe
  source 1..50: mol_type = other DNA
agtggactcattccaattacagggcctcgaaagagtcctgtattgttatt

SEQ ID NO: 61 - DNA, 50 bp, synthetic construct
  misc_feature 1..50: note = DNA Probe
  source 1..50: mol_type = other DNA
cccgggtcgggagtgggtaatttgcgcgcctgctgccttccttggatgtg

SEQ ID NO: 62 - DNA, 50 bp, synthetic construct
  misc_feature 1..50: note = DNA Probe
  source 1..50: mol_type = other DNA
gctccctctccggaatcgaaccctgattccccgtcacccgtggtcaccat

SEQ ID NO: 63 - DNA, 50 bp, synthetic construct
  misc_feature 1..50: note = DNA Probe
  source 1..50: mol_type = other DNA
taccatcgaaagttgatagggcagacgttcgaatgggtcgtcgccgccac

SEQ ID NO: 64 - DNA, 50 bp, synthetic construct
  misc_feature 1..50: note = DNA Probe
  source 1..50: mol_type = other DNA
ggcccgaggttatctagagtcaccaaagccgccggcgcccgccccccggc

SEQ ID NO: 65 - DNA, 50 bp, synthetic construct
  misc_feature 1..50: note = DNA Probe
  source 1..50: mol_type = other DNA
gctgaccgggttggttttgatctgataaatgcacgcatcccccccgcgaa

SEQ ID NO: 66 - DNA, 50 bp, synthetic construct
  misc_feature 1..50: note = DNA Probe
  source 1..50: mol_type = other DNA
tcggcatgtattagctctagaattaccacagttatccaagtaggagagga

SEQ ID NO: 67 - DNA, 50 bp, synthetic construct
  misc_feature 1..50: note = DNA Probe
  source 1..50: mol_type = other DNA
aaccataactgatttaatgagccattcgcagtttcactgtaccggccgtg

SEQ ID NO: 68 - DNA, 50 bp, synthetic construct
  misc_feature 1..50: note = DNA Probe
  source 1..50: mol_type = other DNA
atggcttaatctttgagacaagcatatgctactggcaggatcaaccaggt

SEQ ID NO: 69 - DNA, 50 bp, synthetic construct
  misc_feature 1..50: note = DNA Probe
  source 1..50: mol_type = other DNA
gacaaacccttgtgtcgagggctgactttcaatagatcgcagcgagggag

SEQ ID NO: 70 - DNA, 50 bp, synthetic construct
  misc_feature 1..50: note = DNA Probe
  source 1..50: mol_type = other DNA
cgaaaccccgacccagaagcaggtcgtctacgaatggtttagcgccaggt

SEQ ID NO: 71 - DNA, 50 bp, synthetic construct
  misc_feature 1..50: note = DNA Probe
  source 1..50: mol_type = other DNA
ggtgcgtgacgggcgagggggcggccgcctttccggccgcgccccgtttc

SEQ ID NO: 72 - DNA, 50 bp, synthetic construct
  misc_feature 1..50: note = DNA Probe
  source 1..50: mol_type = other DNA
ctccgcaccggaccccggtcccggcgcgcggcggggcacgcgccctcccg

SEQ ID NO: 73 - DNA, 50 bp, synthetic construct
  misc_feature 1..50: note = DNA Probe
  source 1..50: mol_type = other DNA
aggggggggcggcccgccggcggggacaggcgggggaccggctatccgag

SEQ ID NO: 74 - DNA, 50 bp, synthetic construct
  misc_feature 1..50: note = DNA Probe
  source 1..50: mol_type = other DNA
gcggcgctgccgtatcgttcgcctgggcgggattctgacttagaggcgtt

SEQ ID NO: 75 - DNA, 50 bp, synthetic construct
  misc_feature 1..50: note = DNA Probe
  source 1..50: mol_type = other DNA
agatggtagcttcgccccattggctcctcagccaagcacatacaccaaat

SEQ ID NO: 76 - DNA, 50 bp, synthetic construct
  misc_feature 1..50: note = DNA Probe
  source 1..50: mol_type = other DNA
tcctctcgtactgagcaggattaccatggcaacaacacatcatcagtagg

SEQ ID NO: 77 - DNA, 50 bp, synthetic construct
  misc_feature 1..50: note = DNA Probe
  source 1..50: mol_type = other DNA
ctcacgacggtctaaacccagctcacgttccctattagtgggtgaacaat

SEQ ID NO: 78 - DNA, 50 bp, synthetic construct
  misc_feature 1..50: note = DNA Probe
  source 1..50: mol_type = other DNA
ttctgcttcacaatgataggaagagccgacatcgaaggatcaaaaagcga

SEQ ID NO: 79 - DNA, 50 bp, synthetic construct
  misc_feature 1..50: note = DNA Probe
  source 1..50: mol_type = other DNA
ttggccgccacaagccagttatccctgtggtaacttttctgacacctcct

SEQ ID NO: 80 - DNA, 50 bp, synthetic construct
  misc_feature 1..50: note = DNA Probe
  source 1..50: mol_type = other DNA
ggtcagaaggatcgtgaggccccgctttcacggtctgtattcgtactgaa

SEQ ID NO: 81 - DNA, 50 bp, synthetic construct
  misc_feature 1..50: note = DNA Probe
  source 1..50: mol_type = other DNA
agcttttgcccttctgctccacgggaggtttctgtcctccctgagctcgc

SEQ ID NO: 82 - DNA, 50 bp, synthetic construct
  misc_feature 1..50: note = DNA Probe
  source 1..50: mol_type = other DNA
ttaccgtttgacaggtgtaccgccccagtcaaactccccacctggcactg

SEQ ID NO: 83 - DNA, 50 bp, synthetic construct
  misc_feature 1..50: note = DNA Probe
  source 1..50: mol_type = other DNA
gcgcccggccgggcgggcgcttggcgccagaagcgagagcccctcgggct

SEQ ID NO: 84 - DNA, 50 bp, synthetic construct
  misc_feature 1..50: note = DNA Probe
  source 1..50: mol_type = other DNA
ccgggtcagtgaaaaaacgatcagagtagtggtatttcaccggcggcccg

SEQ ID NO: 85 - DNA, 50 bp, synthetic construct
  misc_feature 1..50: note = DNA Probe
  source 1..50: mol_type = other DNA
cgccccgggcccctcgcggggacaccgggggggcgccgggggcctcccac

SEQ ID NO: 86 - DNA, 50 bp, synthetic construct
  misc_feature 1..50: note = DNA Probe
  source 1..50: mol_type = other DNA
catgtctcttcaccgtgccagactagagtcaagctcaacagggtcttctt

SEQ ID NO: 87 - DNA, 50 bp, synthetic construct
  misc_feature 1..50: note = DNA Probe
  source 1..50: mol_type = other DNA
ccaagcccgttcccttggctgtggtttcgctggatagtaggtagggacag

SEQ ID NO: 88 - DNA, 50 bp, synthetic construct
  misc_feature 1..50: note = DNA Probe
  source 1..50: mol_type = other DNA
tccattcatgcgcgtcactaattagatgacgaggcatttggctaccttaa

SEQ ID NO: 89 - DNA, 50 bp, synthetic construct
  misc_feature 1..50: note = DNA Probe
  source 1..50: mol_type = other DNA
tcccgccgtttacccgcgcttcattgaatttcttcactttgacattcaga

SEQ ID NO: 90 - DNA, 50 bp, synthetic construct
  misc_feature 1..50: note = DNA Probe
  source 1..50: mol_type = other DNA
cacatcgcgtcaacacccgccgcgggccttcgcgatgctttgttttaatt

SEQ ID NO: 91 - DNA, 50 bp, synthetic construct
  misc_feature 1..50: note = DNA Probe
  source 1..50: mol_type = other DNA
cctggtccgcaccagttctaagtcggctgctaggcgccggccgaggcgag

SEQ ID NO: 92 - DNA, 50 bp, synthetic construct
  misc_feature 1..50: note = DNA Probe
  source 1..50: mol_type = other DNA
cggccccgggggcggacccggcgggggggaccggcccgcggcccctccgc

SEQ ID NO: 93 - DNA, 50 bp, synthetic construct
  misc_feature 1..50: note = DNA Probe
  source 1..50: mol_type = other DNA
ccgccgcgcgccgaggaggaggggggaacggggggcggacggggccgggg

SEQ ID NO: 94 - DNA, 50 bp, synthetic construct
  misc_feature 1..50: note = DNA Probe
  source 1..50: mol_type = other DNA
acgaaccgccccgccccgccgcccgccgaccgccgccgcccgaccgctcc

SEQ ID NO: 95 - DNA, 50 bp, synthetic construct
  misc_feature 1..50: note = DNA Probe
  source 1..50: mol_type = other DNA
cgcgcgcgaccgagacgtggggtgggggtggggggcgcgccgcgccgccg

SEQ ID NO: 96 - DNA, 50 bp, synthetic construct
  misc_feature 1..50: note = DNA Probe
  source 1..50: mol_type = other DNA
gcggccgcgacgcccgccgcagctggggcgatccacgggaagggcccggc

SEQ ID NO: 97 - DNA, 50 bp, synthetic construct
  misc_feature 1..50: note = DNA Probe
  source 1..50: mol_type = other DNA
gcgccgccgccggccccccgggtccccggggcccccctcgcggggacctg

SEQ ID NO: 98 - DNA, 50 bp, synthetic construct
  misc_feature 1..50: note = DNA Probe
  source 1..50: mol_type = other DNA
ccggcggccgccgcgcggcccctgccgccccgacccttctccccccgccg

SEQ ID NO: 99 - DNA, 50 bp, synthetic construct
  misc_feature 1..50: note = DNA Probe
  source 1..50: mol_type = other DNA
ctcccccggggaggggggaggacggggagcgggggagagagagagagaga

SEQ ID NO: 100 - DNA, 50 bp, synthetic construct
  misc_feature 1..50: note = DNA Probe
  source 1..50: mol_type = other DNA
agggagcgagcggcgcgcgcgggtggggcgggggagggccgcgagggggg

SEQ ID NO: 101 - DNA, 50 bp, synthetic construct
  misc_feature 1..50: note = DNA Probe
  source 1..50: mol_type = other DNA
gggggcgcgcgcctcgtccagccgcggcgcgcgcccagccccgcttcgcg

SEQ ID NO: 102 - DNA, 50 bp, synthetic construct
  misc_feature 1..50: note = DNA Probe
  source 1..50: mol_type = other DNA
cccagcccttagagccaatccttatcccgaagttacggatccggcttgcc

SEQ ID NO: 103 - DNA, 50 bp, synthetic construct
  misc_feature 1..50: note = DNA Probe
  source 1..50: mol_type = other DNA
cattgttccaacatgccagaggctgttcaccttggagacctgctgcggat

SEQ ID NO: 104 - DNA, 50 bp, synthetic construct
  misc_feature 1..50: note = DNA Probe
  source 1..50: mol_type = other DNA
cgcgagatttacaccctctcccccggattttcaagggccagcgagagctc

SEQ ID NO: 105 - DNA, 50 bp, synthetic construct
  misc_feature 1..50: note = DNA Probe
  source 1..50: mol_type = other DNA
aaccgcgacgctttccaaggcacgggcccctctctcggggcgaacccatt

SEQ ID NO: 106 - DNA, 50 bp, synthetic construct
  misc_feature 1..50: note = DNA Probe
  source 1..50: mol_type = other DNA
cttcacaaagaaaagagaactctccccggggctcccgccggcttctccgg

SEQ ID NO: 107 - DNA, 50 bp, synthetic construct
  misc_feature 1..50: note = DNA Probe
  source 1..50: mol_type = other DNA
cgcactggacgcctcgcggcgcccatctccgccactccggattcggggat

SEQ ID NO: 108 - DNA, 50 bp, synthetic construct
  misc_feature 1..50: note = DNA Probe
  source 1..50: mol_type = other DNA
tttcgatcggccgagggcaacggaggccatcgcccgtcccttcggaacgg

SEQ ID NO: 109 - DNA, 50 bp, synthetic construct
  misc_feature 1..50: note = DNA Probe
  source 1..50: mol_type = other DNA
caggaccgactgacccatgttcaactgctgttcacatggaacccttctcc

SEQ ID NO: 110 - DNA, 50 bp, synthetic construct
  misc_feature 1..50: note = DNA Probe
  source 1..50: mol_type = other DNA
gttctcgtttgaatatttgctactaccaccaagatctgcacctgcggcgg

SEQ ID NO: 111 - DNA, 50 bp, synthetic construct
  misc_feature 1..50: note = DNA Probe
  source 1..50: mol_type = other DNA
cgccctaggcttcaaggctcaccgcagcggccctcctactcgtcgcggcg

SEQ ID NO: 112 - DNA, 50 bp, synthetic construct
  misc_feature 1..50: note = DNA Probe
  source 1..50: mol_type = other DNA
tccgggggcggggagcggggcgtgggcgggaggaggggaggaggcgtggg

SEQ ID NO: 113 - DNA, 50 bp, synthetic construct
  misc_feature 1..50: note = DNA Probe
  source 1..50: mol_type = other DNA
aggaccccacacccccgccgccgccgccgccgccgccctccgacgcacac

SEQ ID NO: 114 - DNA, 50 bp, synthetic construct
  misc_feature 1..50: note = DNA Probe
  source 1..50: mol_type = other DNA
gcgcgccgcccccgccgctcccgtccactctcgactgccggcgacggccg

SEQ ID NO: 115 - DNA, 50 bp, synthetic construct
  misc_feature 1..50: note = DNA Probe
  source 1..50: mol_type = other DNA
ctccagcgccatccattttcagggctagttgattcggcaggtgagttgtt

SEQ ID NO: 116 - DNA, 50 bp, synthetic construct
  misc_feature 1..50: note = DNA Probe
  source 1..50: mol_type = other DNA
gattccgacttccatggccaccgtcctgctgtctatatcaaccaacacct

SEQ ID NO: 117 - DNA, 50 bp, synthetic construct
  misc_feature 1..50: note = DNA Probe
  source 1..50: mol_type = other DNA
gagcgtcggcatcgggcgccttaacccggcgttcggttcatcccgcagcg

SEQ ID NO: 118 - DNA, 50 bp, synthetic construct
  misc_feature 1..50: note = DNA Probe
  source 1..50: mol_type = other DNA
aaaagtggcccactaggcactcgcattccacgcccggctccacgccagcg

SEQ ID NO: 119 - DNA, 50 bp, synthetic construct
  misc_feature 1..50: note = DNA Probe
  source 1..50: mol_type = other DNA
ccatttaaagtttgagaataggttgagatcgtttcggccccaagacctct

SEQ ID NO: 120 - DNA, 50 bp, synthetic construct
  misc_feature 1..50: note = DNA Probe
  source 1..50: mol_type = other DNA
cggataaaactgcgtggcgggggtgcgtcgggtctgcgagagcgccagct

SEQ ID NO: 121 - DNA, 50 bp, synthetic construct
  misc_feature 1..50: note = DNA Probe
  source 1..50: mol_type = other DNA
tcggagggaaccagctactagatggttcgattagtctttcgcccctatac

SEQ ID NO: 122 - DNA, 50 bp, synthetic construct
  misc_feature 1..50: note = DNA Probe
  source 1..50: mol_type = other DNA
gatttgcacgtcaggaccgctacggacctccaccagagtttcctctggct

SEQ ID NO: 123 - DNA, 50 bp, synthetic construct
  misc_feature 1..50: note = DNA Probe
  source 1..50: mol_type = other DNA
atagttcaccatctttcgggtcctaacacgtgcgctcgtgctccacctcc

SEQ ID NO: 124 - DNA, 50 bp, synthetic construct
  misc_feature 1..50: note = DNA Probe
  source 1..50: mol_type = other DNA
agacgggccggtggtgcgccctcggcggactggagaggcctcgggatccc

SEQ ID NO: 125 - DNA, 50 bp, synthetic construct
  misc_feature 1..50: note = DNA Probe
  source 1..50: mol_type = other DNA
cgcgccggccttcaccttcattgcgccacggcggctttcgtgcgagcccc

SEQ ID NO: 126 - DNA, 50 bp, synthetic construct
  misc_feature 1..50: note = DNA Probe
  source 1..50: mol_type = other DNA
ttagactccttggtccgtgtttcaagacgggtcgggtgggtagccgacgt

SEQ ID NO: 127 - DNA, 50 bp, synthetic construct
  misc_feature 1..50: note = DNA Probe
  source 1..50: mol_type = other DNA
gcgctcgctccgccgtccccctcttcgggggacgcgcgcgtggccccgag

SEQ ID NO: 128 - DNA, 50 bp, synthetic construct
  misc_feature 1..50: note = DNA Probe
  source 1..50: mol_type = other DNA
cccgacggcgcgacccgcccggggcgcactggggacagtccgccccgccc

SEQ ID NO: 129 - DNA, 50 bp, synthetic construct
  misc_feature 1..50: note = DNA Probe
  source 1..50: mol_type = other DNA
gcaccccccccgtcgccggggcgggggcgcggggaggaggggtgggagag

SEQ ID NO: 130 - DNA, 50 bp, synthetic construct
  misc_feature 1..50: note = DNA Probe
  source 1..50: mol_type = other DNA
aggggtggcccggcccccccacgaggagacgccggcgcgcccccgcgggg

SEQ ID NO: 131 - DNA, 50 bp, synthetic construct
  misc_feature 1..50: note = DNA Probe
  source 1..50: mol_type = other DNA
ggggattccccgcgggggtgggcgccgggaggggggagagcgcggcgacg

SEQ ID NO: 132 - DNA, 50 bp, synthetic construct
  misc_feature 1..50: note = DNA Probe
  source 1..50: mol_type = other DNA
gccccgggattcggcgagtgctgctgccgggggggctgtaacactcgggg

SEQ ID NO: 133 - DNA, 50 bp, synthetic construct
  misc_feature 1..50: note = DNA Probe
  source 1..50: mol_type = other DNA
ccgcccccgccgccgccgccaccgccgccgccgccgccgccccgacccgc

SEQ ID NO: 134 - DNA, 50 bp, synthetic construct
  misc_feature 1..50: note = DNA Probe
  source 1..50: mol_type = other DNA
aggacgcggggccggggggcggagacgggggaggaggaggacggacggac

SEQ ID NO: 135 - DNA, 50 bp, synthetic construct
  misc_feature 1..50: note = DNA Probe
  source 1..50: mol_type = other DNA
agccaccttccccgccgggccttcccagccgtcccggagccggtcgcggc

SEQ ID NO: 136 - DNA, 50 bp, synthetic construct
  misc_feature 1..50: note = DNA Probe
  source 1..50: mol_type = other DNA
aaatgcgcccggcggcggccggtcgccggtcgggggacggtcccccgccg

SEQ ID NO: 137 - DNA, 50 bp, synthetic construct
  misc_feature 1..50: note = DNA Probe
  source 1..50: mol_type = other DNA
ccgcccgcccacccccgcacccgccggagcccgccccctccggggaggag

SEQ ID NO: 138 - DNA, 50 bp, synthetic construct
  misc_feature 1..50: note = DNA Probe
  source 1..50: mol_type = other DNA
gggaagggagggcgggtggaggggtcgggaggaacggggggcgggaaaga

SEQ ID NO: 139 - DNA, 50 bp, synthetic construct
  misc_feature 1..50: note = DNA Probe
  source 1..50: mol_type = other DNA
acacggccggacccgccgccgggttgaatcctccgggcggactgcgcgga

SEQ ID NO: 140 - DNA, 50 bp, synthetic construct
  misc_feature 1..50: note = DNA Probe
  source 1..50: mol_type = other DNA
tcttaacggtttcacgccctcttgaactctctcttcaaagttcttttcaa

SEQ ID NO: 141 - DNA, 50 bp, synthetic construct
  misc_feature 1..50: note = DNA Probe
  source 1..50: mol_type = other DNA
cttgttgactatcggtctcgtgccggtatttagccttagatggagtttac

SEQ ID NO: 142 - DNA, 50 bp, synthetic construct
  misc_feature 1..50: note = DNA Probe
  source 1..50: mol_type = other DNA
gcattcccaagcaacccgactccgggaagacccgggcgcgcgccggccgc

SEQ ID NO: 143 - DNA, 50 bp, synthetic construct
  misc_feature 1..50: note = DNA Probe
  source 1..50: mol_type = other DNA
gtccacgggctgggcctcgatcagaaggacttgggccccccacgagcggc

SEQ ID NO: 144 - DNA, 50 bp, synthetic construct
  misc_feature 1..50: note = DNA Probe
  source 1..50: mol_type = other DNA
ttccgtacgccacatgtcccgcgccccgcggggcggggattcggcgctgg

SEQ ID NO: 145 - DNA, 50 bp, synthetic construct
  misc_feature 1..50: note = DNA Probe
  source 1..50: mol_type = other DNA
ctcgccgttactgagggaatcctggttagtttcttttcctccgctgacta

SEQ ID NO: 146 - DNA, 50 bp, synthetic construct
  misc_feature 1..50: note = DNA Probe
  source 1..50: mol_type = other DNA
gcgggtcgccacgtctgatctgaggtcgcgtctcggagggggacgggccg

SEQ ID NO: 147 - DNA, 50 bp, synthetic construct
  misc_feature 1..50: note = DNA Probe
  source 1..50: mol_type = other DNA
aagcgacgctcagacaggcgtagccccgggaggaacccggggccgcaagt

SEQ ID NO: 148 - DNA, 50 bp, synthetic construct
  misc_feature 1..50: note = DNA Probe
  source 1..50: mol_type = other DNA
gcagctagctgcgttcttcatcgacgcacgagccgagtgatccaccgcta

SEQ ID NO: 149 - DNA, 50 bp, synthetic construct
  misc_feature 1..50: note = DNA Probe
  source 1..50: mol_type = other DNA
aaagcctacagcacccggtattcccaggcggtctcccatccaagtactaa

SEQ ID NO: 150 - DNA, 50 bp, synthetic construct
  misc_feature 1..50: note = DNA Probe
  source 1..50: mol_type = other DNA
ttccgagatcagacgagatcgggcgcgttcagggtggtatggccgtagac

SEQ ID NO: 151 - DNA, 50 bp, synthetic construct
  misc_feature 1..50: note = DNA Probe
  source 1..50: mol_type = other DNA
gccgcccactcagactttattcaaagaccacgggggtacgggtgcaggaa

SEQ ID NO: 152 - DNA, 50 bp, synthetic construct
  misc_feature 1..50: note = DNA Probe
  source 1..50: mol_type = other DNA
gggggaggcccaaggggcaagaagcatggccaccgaggctccagcttaac

SEQ ID NO: 153 - DNA, 50 bp, synthetic construct
  misc_feature 1..50: note = DNA Probe
  source 1..50: mol_type = other DNA
gcacggtgctcacagaagccaggaacttgtccagggaggcgtgcaccgca

SEQ ID NO: 154 - DNA, 50 bp, synthetic construct
  misc_feature 1..50: note = DNA Probe
  source 1..50: mol_type = other DNA
gggaggtgggcggccagggtcaccagcaggcagtggcttaggagcttgaa

SEQ ID NO: 155 - DNA, 50 bp, synthetic construct
  misc_feature 1..50: note = DNA Probe
  source 1..50: mol_type = other DNA
ccgaagcttgtgcgcgtgcaggtcgctcagggcggacagcgcgttgggca

SEQ ID NO: 156 - DNA, 50 bp, synthetic construct
  misc_feature 1..50: note = DNA Probe
  source 1..50: mol_type = other DNA
ccacggcgttggtcagcgcgtcggccaccttcttgccgtggcccttaacc

SEQ ID NO: 157 - DNA, 50 bp, synthetic construct
  misc_feature 1..50: note = DNA Probe
  source 1..50: mol_type = other DNA
ctcaggtcgaagtgcgggaagtaggtcttggtggtggggaaggacaggaa

SEQ ID NO: 158 - DNA, 50 bp, synthetic construct
  misc_feature 1..50: note = DNA Probe
  source 1..50: mol_type = other DNA
ctccgcaccatactcgccagcgtgcgcgccgaccttaccccaggcggcct

SEQ ID NO: 159 - DNA, 50 bp, synthetic construct
  misc_feature 1..50: note = DNA Probe
  source 1..50: mol_type = other DNA
cggcaggagacagcaccatggtgggttctctctgagtctgtggggaccag

SEQ ID NO: 160 - DNA, 50 bp, synthetic construct
  misc_feature 1..50: note = DNA Probe
  source 1..50: mol_type = other DNA
gaggggaggagggcccgttgggaggcccagcgggcaggaggaacggctac

SEQ ID NO: 161 - DNA, 50 bp, synthetic construct
  misc_feature 1..50: note = DNA Probe
  source 1..50: mol_type = other DNA
acggtatttggaggtcagcacggtgctcacagaagccaggaacttgtcca

SEQ ID NO: 162 - DNA, 50 bp, synthetic construct
  misc_feature 1..50: note = DNA Probe
  source 1..50: mol_type = other DNA
caggggtgaactcggcggggaggtgggcggccagggtcaccagcaggcag

SEQ ID NO: 163 - DNA, 50 bp, synthetic construct
  misc_feature 1..50: note = DNA Probe
  source 1..50: mol_type = other DNA
aagttgaccgggtccacccgaagcttgtgcgcgtgcaggtcgctcagggc

SEQ ID NO: 164 - DNA, 50 bp, synthetic construct
  misc_feature 1..50: note = DNA Probe
  source 1..50: mol_type = other DNA
catgtcgtccacgtgcgccacggcgttggtcagcgcgtcggccaccttct

SEQ ID NO: 165 - DNA, 50 bp, synthetic construct
  misc_feature 1..50: note = DNA Probe
  source 1..50: mol_type = other DNA
cctgggcagagccgtggctcaggtcgaagtgcgggaagtaggtcttggtg

SEQ ID NO: 166 - DNA, 50 bp, synthetic construct
  misc_feature 1..50: note = DNA Probe
  source 1..50: mol_type = other DNA
aacatcctctccagggcctccgcaccatactcgccagcgtgcgcgccgac

SEQ ID NO: 167 - DNA, 50 bp, synthetic construct
  misc_feature 1..50: note = DNA Probe
  source 1..50: mol_type = other DNA
cttgacgttggtcttgtcggcaggagacagcaccatggtgggttctctct

SEQ ID NO: 168 - DNA, 50 bp, synthetic construct
  misc_feature 1..50: note = DNA Probe
  source 1..50: mol_type = other DNA
gcaatgaaaataaatgttttttattaggcagaatccagatgctcaaggcc

SEQ ID NO: 169 - DNA, 50 bp, synthetic construct
  misc_feature 1..50: note = DNA Probe
  source 1..50: mol_type = other DNA
cagtttagtagttggacttagggaacaaaggaacctttaatagaaattgg

SEQ ID NO: 170 - DNA, 50 bp, synthetic construct
  misc_feature 1..50: note = DNA Probe
  source 1..50: mol_type = other DNA
gcttagtgatacttgtgggccagggcattagccacaccagccaccacttt

SEQ ID NO: 171 - DNA, 50 bp, synthetic construct
  misc_feature 1..50: note = DNA Probe
  source 1..50: mol_type = other DNA
cactggtggggtgaattctttgccaaagtgatgggccagcacacagacca

SEQ ID NO: 172 - DNA, 50 bp, synthetic construct
  misc_feature 1..50: note = DNA Probe
  source 1..50: mol_type = other DNA
gcctgaagttctcaggatccacgtgcagcttgtcacagtgcagctcactc

SEQ ID NO: 173 - DNA, 50 bp, synthetic construct
  misc_feature 1..50: note = DNA Probe
  source 1..50: mol_type = other DNA
cccttgaggttgtccaggtgagccaggccatcactaaaggcaccgagcac

SEQ ID NO: 174 - DNA, 50 bp, synthetic construct
  misc_feature 1..50: note = DNA Probe
  source 1..50: mol_type = other DNA
cttcaccttagggttgcccataacagcatcaggagtggacagatccccaa

SEQ ID NO: 175 - DNA, 50 bp, synthetic construct
  misc_feature 1..50: note = DNA Probe
  source 1..50: mol_type = other DNA
tctgggtccaagggtagaccaccagcagcctgcccagggcctcaccacca

SEQ ID NO: 176 - DNA, 50 bp, synthetic construct
  misc_feature 1..50: note = DNA Probe
  source 1..50: mol_type = other DNA
accttgccccacagggcagtaacggcagacttctcctcaggagtcagatg

SEQ ID NO: 177 - DNA, 50 bp, synthetic construct
  misc_feature 1..50: note = DNA Probe
  source 1..50: mol_type = other DNA
gtgatctctcagcagaatagatttattatttgtattgcttgcagaataaa

SEQ ID NO: 178 - DNA, 50 bp, synthetic construct
  misc_feature 1..50: note = DNA Probe
  source 1..50: mol_type = other DNA
ctctgaatcatgggcagtgagctcagtggtatctggaggacagggcactg

SEQ ID NO: 179 - DNA, 50 bp, synthetic construct
  misc_feature 1..50: note = DNA Probe
  source 1..50: mol_type = other DNA
atcttctgccaggaagcctgcacctcaggggtgaattctttgccgaaatg

SEQ ID NO: 180 - DNA, 50 bp, synthetic construct
  misc_feature 1..50: note = DNA Probe
  source 1..50: mol_type = other DNA
caccagcacatttcccaggagcttgaagttctcaggatccacatgcagct

SEQ ID NO: 181 - DNA, 50 bp, synthetic construct
  misc_feature 1..50: note = DNA Probe
  source 1..50: mol_type = other DNA
cactcagctgggcaaaggtgcccttgagatcatccaggtgctttgtggca

SEQ ID NO: 182 - DNA, 50 bp, synthetic construct
  misc_feature 1..50: note = DNA Probe
  source 1..50: mol_type = other DNA
agcaccttcttgccatgtgccttgactttggggttgcccatgatggcaga

SEQ ID NO: 183 - DNA, 50 bp, synthetic construct
  misc_feature 1..50: note = DNA Probe
  source 1..50: mol_type = other DNA
gccaaagctgtcaaagaacctctgggtccatgggtagacaaccaggagcc

SEQ ID NO: 184 - DNA, 50 bp, synthetic construct
  misc_feature 1..50: note = DNA Probe
  source 1..50: mol_type = other DNA
ctccagcatcttccacattcaccttgccccacaggcttgtgatagtagcc

SEQ ID NO: 185 - DNA, 50 bp, synthetic construct
  misc_feature 1..50: note = DNA Probe
  source 1..50: mol_type = other DNA
aaatgacccatggcgtctggactaggagcttattgataacctcagacgtt

SEQ ID NO: 186 - DNA, 50 bp, synthetic construct
  misc_feature 1..50: note = DNA Probe
  source 1..50: mol_type = other DNA
gtgatctcttagcagaatagatttattatttgattgcttgcagaataaag

SEQ ID NO: 187 - DNA, 50 bp, synthetic construct
  misc_feature 1..50: note = DNA Probe
  source 1..50: mol_type = other DNA
tctgcatcatgggcagtgagctcagtggtatctggaggacagggcactgg

SEQ ID NO: 188 - DNA, 50 bp, synthetic construct
  misc_feature 1..50: note = DNA Probe
  source 1..50: mol_type = other DNA
tcttctgccaggaagcctgcacctcaggggtgaattctttgccgaaatgg

SEQ ID NO: 189 - DNA, 50 bp, synthetic construct
  misc_feature 1..50: note = DNA Probe
  source 1..50: mol_type = other DNA
accagcacatttcccaggagcttgaagttctcaggatccacatgcagctt

SEQ ID NO: 190 - DNA, 50 bp, synthetic construct
  misc_feature 1..50: note = DNA Probe
  source 1..50: mol_type = other DNA
actcagctgggcaaaggtgcccttgagatcatccaggtgctttatggcat

SEQ ID NO: 191 - DNA, 50 bp, synthetic construct
  misc_feature 1..50: note = DNA Probe
  source 1..50: mol_type = other DNA
gcaccttcttgccatgtgccttgactttggggttgcccatgatggcagag

SEQ ID NO: 192 - DNA, 50 bp, synthetic construct
  misc_feature 1..50: note = DNA Probe
  source 1..50: mol_type = other DNA
ccaaagctgtcaaagaacctctgggtccatgggtagacaaccaggagcct

SEQ ID NO: 193 - DNA, 50 bp, synthetic construct
  misc_feature 1..50: note = DNA Probe
  source 1..50: mol_type = other DNA
tccagcatcttccacattcaccttgccccacaggcttgtgatagtagcct

SEQ ID NO: 194 - DNA, 50 bp, synthetic construct
  misc_feature 1..50: note = DNA Probe
  source 1..50: mol_type = other DNA
aatgacccatggcgtctggactaggagcttattgataacctcagacgttc

SEQ ID NO: 195 - DNA, 50 bp, synthetic construct
  misc_feature 1..50: note = DNA Probe
  source 1..50: mol_type = other DNA
atgcctggcagttccctactctcgcatggggagaccccacactaccatcg

SEQ ID NO: 196 - DNA, 50 bp, synthetic construct
  misc_feature 1..50: note = DNA Probe
  source 1..50: mol_type = other DNA
acttctgagttcggcatggggtcaggtgggaccaccgcgctacggccgcc

SEQ ID NO: 197 - DNA, 50 bp, synthetic construct
  misc_feature 1..50: note = DNA Probe
  source 1..50: mol_type = other DNA
ggttaccttgttacgacttcaccccagtcatgaatcacaaagtggtaagt

SEQ ID NO: 198 - DNA, 50 bp, synthetic construct
  misc_feature 1..50: note = DNA Probe
  source 1..50: mol_type = other DNA
aagctacctacttcttttgcaacccactcccatggtgtgacgggcggtgt

SEQ ID NO: 199 - DNA, 50 bp, synthetic construct
  misc_feature 1..50: note = DNA Probe
  source 1..50: mol_type = other DNA
acgtattcaccgtggcattctgatccacgattactagcgattccgacttc

SEQ ID NO: 200 - DNA, 50 bp, synthetic construct
  misc_feature 1..50: note = DNA Probe
  source 1..50: mol_type = other DNA
agactccaatccggactacgacgcactttatgaggtccgcttgctctcgc

SEQ ID NO: 201 - DNA, 50 bp, synthetic construct
  misc_feature 1..50: note = DNA Probe
  source 1..50: mol_type = other DNA
tgtatgcgccattgtagcacgtgtgtagccctggtcgtaagggccatgat

SEQ ID NO: 202 - DNA, 50 bp, synthetic construct
  misc_feature 1..50: note = DNA Probe
  source 1..50: mol_type = other DNA
ccaccttcctccagtttatcactggcagtctcctttgagttcccggccgg

SEQ ID NO: 203 - DNA, 50 bp, synthetic construct
  misc_feature 1..50: note = DNA Probe
  source 1..50: mol_type = other DNA
ggataagggttgcgctcgttgcgggacttaacccaacatttcacaacacg

SEQ ID NO: 204 - DNA, 50 bp, synthetic construct
  misc_feature 1..50: note = DNA Probe
  source 1..50: mol_type = other DNA
tgcagcacctgtctcacggttcccgaaggcacattctcatctctgaaaac

SEQ ID NO: 205 - DNA, 50 bp, synthetic construct
  misc_feature 1..50: note = DNA Probe
  source 1..50: mol_type = other DNA
gaccaggtaaggttcttcgcgttgcatcgaattaaaccacatgctccacc

SEQ ID NO: 206 - DNA, 50 bp, synthetic construct
  misc_feature 1..50: note = DNA Probe
  source 1..50: mol_type = other DNA
cgtcaattcatttgagttttaaccttgcggccgtactccccaggcggtcg

SEQ ID NO: 207 - DNA, 50 bp, synthetic construct
  misc_feature 1..50: note = DNA Probe
  source 1..50: mol_type = other DNA
tccggaagccacgcctcaagggcacaacctccaagtcgacatcgtttacg

SEQ ID NO: 208 - DNA, 50 bp, synthetic construct
  misc_feature 1..50: note = DNA Probe
  source 1..50: mol_type = other DNA
gtatctaatcctgtttgctccccacgctttcgcactgagcgtcagtcttc

SEQ ID NO: 209 - DNA, 50 bp, synthetic construct
  misc_feature 1..50: note = DNA Probe
  source 1..50: mol_type = other DNA
ttcgccaccggtattcctccagatctctacgcatttcaccgctacacctg

SEQ ID NO: 210 - DNA, 50 bp, synthetic construct
  misc_feature 1..50: note = DNA Probe
  source 1..50: mol_type = other DNA
ctacgagactcaagcttgccagtatcagatgcagttcccaggttgagccc

SEQ ID NO: 211 - DNA, 50 bp, synthetic construct
  misc_feature 1..50: note = DNA Probe
  source 1..50: mol_type = other DNA
gacttaacaaaccgcctgcgtgcgctttacgcccagtaattccgattaac

SEQ ID NO: 212 - DNA, 50 bp, synthetic construct
  misc_feature 1..50: note = DNA Probe
  source 1..50: mol_type = other DNA
attaccgcggctgctggcacggagttagccggtgcttcttctgcgggtaa

SEQ ID NO: 213 - DNA, 50 bp, synthetic construct
  misc_feature 1..50: note = DNA Probe
  source 1..50: mol_type = other DNA
gtattaactttactcccttcctccccgctgaaagtactttacaacccgaa

SEQ ID NO: 214 - DNA, 50 bp, synthetic construct
  misc_feature 1..50: note = DNA Probe
  source 1..50: mol_type = other DNA
cgcggcatggctgcatcaggcttgcgcccattgtgcagtattccccactg

SEQ ID NO: 215 - DNA, 50 bp, synthetic construct
  misc_feature 1..50: note = DNA Probe
  source 1..50: mol_type = other DNA
gtctggaccgtgtctcagttccagtgtggctggtcatcctctcagaccag

SEQ ID NO: 216 - DNA, 50 bp, synthetic construct
  misc_feature 1..50: note = DNA Probe
  source 1..50: mol_type = other DNA
taggtgagccgttaccccacctactagctaatcccatctgggcacatccg

SEQ ID NO: 217 - DNA, 50 bp, synthetic construct
  misc_feature 1..50: note = DNA Probe
  source 1..50: mol_type = other DNA
aaggtccccctctttggtcttgcgacgttatgcggtattagctaccgttt

SEQ ID NO: 218 - DNA, 50 bp, synthetic construct
  misc_feature 1..50: note = DNA Probe
  source 1..50: mol_type = other DNA
ctccatcaggcagtttcccagacattactcacccgtccgccactcgtcag

SEQ ID NO: 219 - DNA, 50 bp, synthetic construct
  misc_feature 1..50: note = DNA Probe
  source 1..50: mol_type = other DNA
aaggttaagcctcacggttcattagtaccggttagctcaacgcatcgctg

SEQ ID NO: 220 - DNA, 50 bp, synthetic construct
  misc_feature 1..50: note = DNA Probe
  source 1..50: mol_type = other DNA
cctatcaacgtcgtcgtcttcaacgttccttcaggacccttaaagggtca

SEQ ID NO: 221 - DNA, 50 bp, synthetic construct
  misc_feature 1..50: note = DNA Probe
  source 1..50: mol_type = other DNA
ggggcaagtttcgtgcttagatgctttcagcacttatctcttccgcattt

SEQ ID NO: 222 - DNA, 50 bp, synthetic construct
  misc_feature 1..50: note = DNA Probe
  source 1..50: mol_type = other DNA
ccattggcatgacaacccgaacaccagtgatgcgtccactccggtcctct

SEQ ID NO: 223 - DNA, 50 bp, synthetic construct
  misc_feature 1..50: note = DNA Probe
  source 1..50: mol_type = other DNA
ccccctcagttctccagcgcccacggcagatagggaccgaactgtctcac

SEQ ID NO: 224 - DNA, 50 bp, synthetic construct
  misc_feature 1..50: note = DNA Probe
  source 1..50: mol_type = other DNA
gctcgcgtaccactttaaatggcgaacagccatacccttgggacctactt

SEQ ID NO: 225 - DNA, 50 bp, synthetic construct
  misc_feature 1..50: note = DNA Probe
  source 1..50: mol_type = other DNA
atgagccgacatcgaggtgccaaacaccgccgtcgatatgaactcttggg

SEQ ID NO: 226 - DNA, 50 bp, synthetic construct
  misc_feature 1..50: note = DNA Probe
  source 1..50: mol_type = other DNA
atccccggagtaccttttatccgttgagcgatggcccttccattcagaac

SEQ ID NO: 227 - DNA, 50 bp, synthetic construct
  misc_feature 1..50: note = DNA Probe
  source 1..50: mol_type = other DNA
acctgctttcgcacctgctcgcgccgtcacgctcgcagtcaagctggctt

SEQ ID NO: 228 - DNA, 50 bp, synthetic construct
  misc_feature 1..50: note = DNA Probe
  source 1..50: mol_type = other DNA
cctcctgatgtccgaccaggattagccaaccttcgtgctcctccgttact

SEQ ID NO: 229 - DNA, 50 bp, synthetic construct
  misc_feature 1..50: note = DNA Probe
  source 1..50: mol_type = other DNA
gccccagtcaaactacccaccagacactgtccgcaacccggattacgggt

SEQ ID NO: 230 - DNA, 50 bp, synthetic construct
  misc_feature 1..50: note = DNA Probe
  source 1..50: mol_type = other DNA
aaacattaaagggtggtatttcaaggtcggctccatgcagactggcgtcc

SEQ ID NO: 231 - DNA, 50 bp, synthetic construct
  misc_feature 1..50: note = DNA Probe
  source 1..50: mol_type = other DNA
ccacctatcctacacatcaaggctcaatgttcagtgtcaagctatagtaa

SEQ ID NO: 232 - DNA, 50 bp, synthetic construct
  misc_feature 1..50: note = DNA Probe
  source 1..50: mol_type = other DNA
ttccgtcttgccgcgggtacactgcatcttcacagcgagttcaatttcac

SEQ ID NO: 233 - DNA, 50 bp, synthetic construct
  misc_feature 1..50: note = DNA Probe
  source 1..50: mol_type = other DNA
gacagcctggccatcattacgccattcgtgcaggtcggaacttacccgac

SEQ ID NO: 234 - DNA, 50 bp, synthetic construct
  misc_feature 1..50: note = DNA Probe
  source 1..50: mol_type = other DNA
cttaggaccgttatagttacggccgccgtttaccggggcttcgatcaaga

SEQ ID NO: 235 - DNA, 50 bp, synthetic construct
  misc_feature 1..50: note = DNA Probe
  source 1..50: mol_type = other DNA
accccatcaattaaccttccggcaccgggcaggcgtcacaccgtatacgt

SEQ ID NO: 236 - DNA, 50 bp, synthetic construct
  misc_feature 1..50: note = DNA Probe
  source 1..50: mol_type = other DNA
cacagtgctgtgtttttaataaacagttgcagccagctggtatcttcgac

SEQ ID NO: 237 - DNA, 50 bp, synthetic construct
  misc_feature 1..50: note = DNA Probe
  source 1..50: mol_type = other DNA
ccgcgagggacctcacctacatatcagcgtgccttctcccgaagttacgg

SEQ ID NO: 238 - DNA, 50 bp, synthetic construct
  misc_feature 1..50: note = DNA Probe
  source 1..50: mol_type = other DNA
ttccttcacccgagttctctcaagcgccttggtattctctacctgaccac

SEQ ID NO: 239 - DNA, 50 bp, synthetic construct
  misc_feature 1..50: note = DNA Probe
  source 1..50: mol_type = other DNA
gtacgatttgatgttacctgatgcttagaggcttttcctggaagcagggc

SEQ ID NO: 240 - DNA, 50 bp, synthetic construct
  misc_feature 1..50: note = DNA Probe
  source 1..50: mol_type = other DNA
accgtagtgcctcgtcatcacgcctcagccttgattttccggatttgcct

SEQ ID NO: 241 - DNA, 50 bp, synthetic construct
  misc_feature 1..50: note = DNA Probe
  source 1..50: mol_type = other DNA
acgcttaaaccgggacaaccgtcgcccggccaacatagccttctccgtcc

SEQ ID NO: 242 - DNA, 50 bp, synthetic construct
  misc_feature 1..50: note = DNA Probe
  source 1..50: mol_type = other DNA
accaagtacaggaatattaacctgtttcccatcgactacgcctttcggcc

SEQ ID NO: 243 - DNA, 50 bp, synthetic construct
  misc_feature 1..50: note = DNA Probe
  source 1..50: mol_type = other DNA
actcaccctgccccgattaacgttggacaggaacccttggtcttccggcg

SEQ ID NO: 244 - DNA, 50 bp, synthetic construct
  misc_feature 1..50: note = DNA Probe
  source 1..50: mol_type = other DNA
cgctttatcgttacttatgtcagcattcgcacttctgatacctccagcat

SEQ ID NO: 245 - DNA, 50 bp, synthetic construct
  misc_feature 1..50: note = DNA Probe
  source 1..50: mol_type = other DNA
ttcgcaggcttacagaacgctcccctacccaacaacgcataagcgtcgct

SEQ ID NO: 246 - DNA, 50 bp, synthetic construct
  misc_feature 1..50: note = DNA Probe
  source 1..50: mol_type = other DNA
catggtttagccccgttacatcttccgcgcaggccgactcgaccagtgag

SEQ ID NO: 247 - DNA, 50 bp, synthetic construct
  misc_feature 1..50: note = DNA Probe
  source 1..50: mol_type = other DNA
taaatgatggctgcttctaagccaacatcctggctgtctgggccttccca

SEQ ID NO: 248 - DNA, 50 bp, synthetic construct
  misc_feature 1..50: note = DNA Probe
  source 1..50: mol_type = other DNA
aaccatgactttgggaccttagctggcggtctgggttgtttccctcttca

SEQ ID NO: 249 - DNA, 50 bp, synthetic construct
  misc_feature 1..50: note = DNA Probe
  source 1..50: mol_type = other DNA
cccgccgtgtgtctcccgtgataacattctccggtattcgcagtttgcat

SEQ ID NO: 250 - DNA, 50 bp, synthetic construct
  misc_feature 1..50: note = DNA Probe
  source 1..50: mol_type = other DNA
ggatgacccccttgccgaaacagtgctctacccccggagatgaattcacg

SEQ ID NO: 251 - DNA, 50 bp, synthetic construct
  misc_feature 1..50: note = DNA Probe
  source 1..50: mol_type = other DNA
agctttcggggagaaccagctatctcccggtttgattggcctttcacccc

SEQ ID NO: 252 - DNA, 50 bp, synthetic construct
  misc_feature 1..50: note = DNA Probe
  source 1..50: mol_type = other DNA
cgctaatttttcaacattagtcggttcggtcctccagttagtgttaccca

SEQ ID NO: 253 - DNA, 50 bp, synthetic construct
  misc_feature 1..50: note = DNA Probe
  source 1..50: mol_type = other DNA
atggctagatcaccgggtttcgggtctataccctgcaacttaacgcccag

SEQ ID NO: 254 - DNA, 50 bp, synthetic construct
  misc_feature 1..50: note = DNA Probe
  source 1..50: mol_type = other DNA
ccttcggctcccctattcggttaaccttgctacagaatataagtcgctga

SEQ ID NO: 255 - DNA, 50 bp, synthetic construct
  misc_feature 1..50: note = DNA Probe
  source 1..50: mol_type = other DNA
gtacgcagtcacacgcctaagcgtgctcccactgcttgtacgtacacggt

SEQ ID NO: 256 - DNA, 50 bp, synthetic construct
  misc_feature 1..50: note = DNA Probe
  source 1..50: mol_type = other DNA
actcccctcgccggggttcttttcgcctttccctcacggtactggttcac

SEQ ID NO: 257 - DNA, 50 bp, synthetic construct
  misc_feature 1..50: note = DNA Probe
  source 1..50: mol_type = other DNA
agtatttagccttggaggatggtccccccatattcagacaggataccacg

SEQ ID NO: 258 - DNA, 50 bp, synthetic construct
  misc_feature 1..50: note = DNA Probe
  source 1..50: mol_type = other DNA
atcgagctcacagcatgtgcatttttgtgtacggggctgtcaccctgtat

SEQ ID NO: 259 - DNA, 50 bp, synthetic construct
  misc_feature 1..50: note = DNA Probe
  source 1..50: mol_type = other DNA
acgcttccactaacacacacactgattcaggctctgggctgctccccgtt

SEQ ID NO: 260 - DNA, 50 bp, synthetic construct
  misc_feature 1..50: note = DNA Probe
  source 1..50: mol_type = other DNA
ggggaatctcggttgatttcttttcctcggggtacttagatgtttcagtt

SEQ ID NO: 261 - DNA, 50 bp, synthetic construct
  misc_feature 1..50: note = DNA Probe
  source 1..50: mol_type = other DNA
attaacctatggattcagttaatgatagtgtgtcgaaacacactgggttt

SEQ ID NO: 262 - DNA, 50 bp, synthetic construct
  misc_feature 1..50: note = DNA Probe
  source 1..50: mol_type = other DNA
gccggttataacggttcatatcaccttaccgacgcttatcgcagattagc

SEQ ID NO: 263 - DNA, 50 bp, synthetic construct
  misc_feature 1..50: note = DNA Probe
  source 1..50: mol_type = other DNA
gcttggcggcgtcctactctcacagggggaaacccccgactaccatcggc

SEQ ID NO: 264 - DNA, 50 bp, synthetic construct
  misc_feature 1..50: note = DNA Probe
  source 1..50: mol_type = other DNA
ttccgtgttcggtatgggaacgggtgtgacctcttcgctatcgccaccaa

SEQ ID NO: 265 - DNA, 50 bp, synthetic construct
  misc_feature 1..50: note = DNA Probe
  source 1..50: mol_type = other DNA
tagaaaggaggtgatccagccgcaccttccgatacggctaccttgttacg

SEQ ID NO: 266 - DNA, 50 bp, synthetic construct
  misc_feature 1..50: note = DNA Probe
  source 1..50: mol_type = other DNA
tctgtcccaccttcggcggctggctcctaaaaggttacctcaccgacttc

SEQ ID NO: 267 - DNA, 50 bp, synthetic construct
  misc_feature 1..50: note = DNA Probe
  source 1..50: mol_type = other DNA
tcgtggtgtgacgggcggtgtgtacaaggcccgggaacgtattcaccgcg

SEQ ID NO: 268 - DNA, 50 bp, synthetic construct
  misc_feature 1..50: note = DNA Probe
  source 1..50: mol_type = other DNA
attactagcgattccagcttcacgcagtcgagttgcagactgcgatccga

SEQ ID NO: 269 - DNA, 50 bp, synthetic construct
  misc_feature 1..50: note = DNA Probe
  source 1..50: mol_type = other DNA
gtgggattggcttaacctcgcggtttcgctgccctttgttctgtccattg

SEQ ID NO: 270 - DNA, 50 bp, synthetic construct
  misc_feature 1..50: note = DNA Probe
  source 1..50: mol_type = other DNA
ccaggtcataaggggcatgatgatttgacgtcatccccaccttcctccgg

SEQ ID NO: 271 - DNA, 50 bp, synthetic construct
  misc_feature 1..50: note = DNA Probe
  source 1..50: mol_type = other DNA
caccttagagtgcccaactgaatgctggcaactaagatcaagggttgcgc

SEQ ID NO: 272 - DNA, 50 bp, synthetic construct
  misc_feature 1..50: note = DNA Probe
  source 1..50: mol_type = other DNA
acccaacatctcacgacacgagctgacgacaaccatgcaccacctgtcac

SEQ ID NO: 273 - DNA, 50 bp, synthetic construct
  misc_feature 1..50: note = DNA Probe
  source 1..50: mol_type = other DNA
gacgtcctatctctaggattgtcagaggatgtcaagacctggtaaggttc

SEQ ID NO: 274 - DNA, 50 bp, synthetic construct
  misc_feature 1..50: note = DNA Probe
  source 1..50: mol_type = other DNA
attaaaccacatgctccaccgcttgtgcgggcccccgtcaattcctttga

SEQ ID NO: 275 - DNA, 50 bp, synthetic construct
  misc_feature 1..50: note = DNA Probe
  source 1..50: mol_type = other DNA
ccgtactccccaggcggagtgcttaatgcgttagctgcagcactaagggg

SEQ ID NO: 276 - DNA, 50 bp, synthetic construct
  misc_feature 1..50: note = DNA Probe
  source 1..50: mol_type = other DNA
acttagcactcatcgtttacggcgtggactaccagggtatctaatcctgt

SEQ ID NO: 277 - DNA, 50 bp, synthetic construct
  misc_feature 1..50: note = DNA Probe
  source 1..50: mol_type = other DNA
tcgctcctcagcgtcagttacagaccagagagtcgccttcgccactggtg

SEQ ID NO: 278 - DNA, 50 bp, synthetic construct
  misc_feature 1..50: note = DNA Probe
  source 1..50: mol_type = other DNA
acgcatttcaccgctacacgtggaattccactctcctcttctgcactcaa

SEQ ID NO: 279 - DNA, 50 bp, synthetic construct
  misc_feature 1..50: note = DNA Probe
  source 1..50: mol_type = other DNA
atgaccctccccggttgagccgggggctttcacatcagacttaagaaacc

SEQ ID NO: 280 - DNA, 50 bp, synthetic construct
  misc_feature 1..50: note = DNA Probe
  source 1..50: mol_type = other DNA
acgcccaataattccggacaacgcttgccacctacgtattaccgcggctg

SEQ ID NO: 281 - DNA, 50 bp, synthetic construct
  misc_feature 1..50: note = DNA Probe
  source 1..50: mol_type = other DNA
ccgtggctttctggttaggtaccgtcaaggtaccgccctattcgaacggt

SEQ ID NO: 282 - DNA, 50 bp, synthetic construct
  misc_feature 1..50: note = DNA Probe
  source 1..50: mol_type = other DNA
acaacagagctttacgatccgaaaaccttcatcactcacgcggcgttgct

SEQ ID NO: 283 - DNA, 50 bp, synthetic construct
  misc_feature 1..50: note = DNA Probe
  source 1..50: mol_type = other DNA
ccattgcggaagattccctactgctgcctcccgtaggagtctgggccgtg

SEQ ID NO: 284 - DNA, 50 bp, synthetic construct
  misc_feature 1..50: note = DNA Probe
  source 1..50: mol_type = other DNA
ggccgatcaccctctcaggtcggctacgcatcgtcgccttggtgagccgt

SEQ ID NO: 285 - DNA, 50 bp, synthetic construct
  misc_feature 1..50: note = DNA Probe
  source 1..50: mol_type = other DNA
ctaatgcgccgcgggtccatctgtaagtggtagccgaagccaccttttat

SEQ ID NO: 286 - DNA, 50 bp, synthetic construct
  misc_feature 1..50: note = DNA Probe
  source 1..50: mol_type = other DNA
ttcaaacaaccatccggtattagccccggtttcccggagttatcccagtc

SEQ ID NO: 287 - DNA, 50 bp, synthetic construct
  misc_feature 1..50: note = DNA Probe
  source 1..50: mol_type = other DNA
ccacgtgttactcacccgtccgccgctaacatcagggagcaagctcccat

SEQ ID NO: 288 - DNA, 50 bp, synthetic construct
  misc_feature 1..50: note = DNA Probe
  source 1..50: mol_type = other DNA
gcatgtattaggcacgccgccagcgttcgtcctgagccaggatcaaactc

SEQ ID NO: 289 - DNA, 50 bp, synthetic construct
  misc_feature 1..50: note = DNA Probe
  source 1..50: mol_type = other DNA
tggttaagtcctcgatcgattagtatctgtcagctccatgtgtcgccaca

SEQ ID NO: 290 - DNA, 50 bp, synthetic construct
  misc_feature 1..50: note = DNA Probe
  source 1..50: mol_type = other DNA
tatcaacctgatcatctttcagggatcttacttccttgcggaatgggaaa

SEQ ID NO: 291 - DNA, 50 bp, synthetic construct
  misc_feature 1..50: note = DNA Probe
  source 1..50: mol_type = other DNA
ggcttcatgcttagatgctttcagcacttatcccgtccgcacatagctac

SEQ ID NO: 292 - DNA, 50 bp, synthetic construct
  misc_feature 1..50: note = DNA Probe
  source 1..50: mol_type = other DNA
gcagaacaactggtacaccagcggtgcgtccatcccggtcctctcgtact

SEQ ID NO: 293 - DNA, 50 bp, synthetic construct
  misc_feature 1..50: note = DNA Probe
  source 1..50: mol_type = other DNA
caaatttcctgcgcccgcgacggatagggaccgaactgtctcacgacgtt

SEQ ID NO: 294 - DNA, 50 bp, synthetic construct
  misc_feature 1..50: note = DNA Probe
  source 1..50: mol_type = other DNA
gtaccgctttaatgggcgaacagcccaacccttgggactgactacagccc

SEQ ID NO: 295 - DNA, 50 bp, synthetic construct
  misc_feature 1..50: note = DNA Probe
  source 1..50: mol_type = other DNA
cgacatcgaggtgccaaacctccccgtcgatgtggactcttgggggagat

SEQ ID NO: 296 - DNA, 50 bp, synthetic construct
  misc_feature 1..50: note = DNA Probe
  source 1..50: mol_type = other DNA
ggggtagcttttatccgttgagcgatggcccttccatgcggaaccaccgg

SEQ ID NO: 297 - DNA, 50 bp, synthetic construct
  misc_feature 1..50: note = DNA Probe
  source 1..50: mol_type = other DNA
tttcgtccctgctcgacttgtaggtctcgcagtcaagctcccttgtgcct

SEQ ID NO: 298 - DNA, 50 bp, synthetic construct
  misc_feature 1..50: note = DNA Probe
  source 1..50: mol_type = other DNA
gatttccaaccattctgagggaacctttgggcgcctccgttaccttttag

SEQ ID NO: 299 - DNA, 50 bp, synthetic construct
  misc_feature 1..50: note = DNA Probe
  source 1..50: mol_type = other DNA
gtcaaactgcccacctgacactgtctccccgcccgataagggcggcgggt

SEQ ID NO: 300 - DNA, 50 bp, synthetic construct
  misc_feature 1..50: note = DNA Probe
  source 1..50: mol_type = other DNA
gccagggtagtatcccaccgatgcctccaccgaagctggcgctccggttt

SEQ ID NO: 301 - DNA, 50 bp, synthetic construct
  misc_feature 1..50: note = DNA Probe
  source 1..50: mol_type = other DNA
atcctgtacaagctgtaccaacattcaatatcaggctgcagtaaagctcc

SEQ ID NO: 302 - DNA, 50 bp, synthetic construct
  misc_feature 1..50: note = DNA Probe
  source 1..50: mol_type = other DNA
cctgtcgcgggtaacctgcatcttcacaggtactataatttcaccgagtc

SEQ ID NO: 303 - DNA, 50 bp, synthetic construct
  misc_feature 1..50: note = DNA Probe
  source 1..50: mol_type = other DNA
gcccagatcgttgcgcctttcgtgcgggtcggaacttacccgacaaggaa

SEQ ID NO: 304 - DNA, 50 bp, synthetic construct
  misc_feature 1..50: note = DNA Probe
  source 1..50: mol_type = other DNA
accgttatagttacggccgccgtttactggggcttcaattcgcaccttcg

SEQ ID NO: 305 - DNA, 50 bp, synthetic construct
  misc_feature 1..50: note = DNA Probe
  source 1..50: mol_type = other DNA
cctcttaaccttccagcaccgggcaggcgtcagcccctatacttcgcctt

SEQ ID NO: 306 - DNA, 50 bp, synthetic construct
  misc_feature 1..50: note = DNA Probe
  source 1..50: mol_type = other DNA
cctgtgtttttgctaaacagtcgcctgggcctattcactgcggctctctc

SEQ ID NO: 307 - DNA, 50 bp, synthetic construct
  misc_feature 1..50: note = DNA Probe
  source 1..50: mol_type = other DNA
cagagcaccccttctcccgaagttacggggtcattttgccgagttcctta

SEQ ID NO: 308 - DNA, 50 bp, synthetic construct
  misc_feature 1..50: note = DNA Probe
  source 1..50: mol_type = other DNA
atcaccttaggattctctcctcgcctacctgtgtcggtttgcggtacggg

SEQ ID NO: 309 - DNA, 50 bp, synthetic construct
  misc_feature 1..50: note = DNA Probe
  source 1..50: mol_type = other DNA
tagaggcttttcttggcagtgtggaatcaggaacttcgctactatatttc

SEQ ID NO: 310 - DNA, 50 bp, synthetic construct
  misc_feature 1..50: note = DNA Probe
  source 1..50: mol_type = other DNA
tcagccttatgggaaacggatttgcctatttcccagcctaactgcttgga

SEQ ID NO: 311 - DNA, 50 bp, synthetic construct
  misc_feature 1..50: note = DNA Probe
  source 1..50: mol_type = other DNA
ccgcgcttaccctatcctcctgcgtccccccattgctcaaatggtgagga

SEQ ID NO: 312 - DNA, 50 bp, synthetic construct
  misc_feature 1..50: note = DNA Probe
  source 1..50: mol_type = other DNA
tcaacctgttgtccatcgcctacgcctttcggcctcggcttaggtcccga

SEQ ID NO: 313 - DNA, 50 bp, synthetic construct
  misc_feature 1..50: note = DNA Probe
  source 1..50: mol_type = other DNA
cgagccttcctcaggaaaccttaggcattcggtggaggggattctcaccc

SEQ ID NO: 314 - DNA, 50 bp, synthetic construct
  misc_feature 1..50: note = DNA Probe
  source 1..50: mol_type = other DNA
taccggcattctcacttctaagcgctccaccagtccttccggtctggctt

SEQ ID NO: 315 - DNA, 50 bp, synthetic construct
  misc_feature 1..50: note = DNA Probe
  source 1..50: mol_type = other DNA
gctctcctaccactgttcgaagaacagtccgcagcttcggtgatacgttt

SEQ ID NO: 316 - DNA, 50 bp, synthetic construct
  misc_feature 1..50: note = DNA Probe
  source 1..50: mol_type = other DNA
tcggcgcagagtcactcgaccagtgagctattacgcactctttaaatggt

SEQ ID NO: 317 - DNA, 50 bp, synthetic construct
  misc_feature 1..50: note = DNA Probe
  source 1..50: mol_type = other DNA
aacatcctggttgtctaagcaactccacatccttttccacttaacgtata

SEQ ID NO: 318 - DNA, 50 bp, synthetic construct
  misc_feature 1..50: note = DNA Probe
  source 1..50: mol_type = other DNA
tggcggtctgggctgtttccctttcgactacggatcttatcactcgcagt

SEQ ID NO: 319 - DNA, 50 bp, synthetic construct
  misc_feature 1..50: note = DNA Probe
  source 1..50: mol_type = other DNA
aagtcattggcattcggagtttgactgaattcggtaacccggtaggggcc

SEQ ID NO: 320 - DNA, 50 bp, synthetic construct
  misc_feature 1..50: note = DNA Probe
  source 1..50: mol_type = other DNA
gctctacctccaagactcttaccttgaggctagccctaaagctatttcgg

SEQ ID NO: 321 - DNA, 50 bp, synthetic construct
  misc_feature 1..50: note = DNA Probe
  source 1..50: mol_type = other DNA
tccaggttcgattggcatttcacccctacccacacctcatccccgcactt

SEQ ID NO: 322 - DNA, 50 bp, synthetic construct
  misc_feature 1..50: note = DNA Probe
  source 1..50: mol_type = other DNA
ttcgggcctccattcagtgttacctgaacttcaccctggacatgggtaga

SEQ ID NO: 323 - DNA, 50 bp, synthetic construct
  misc_feature 1..50: note = DNA Probe
  source 1..50: mol_type = other DNA
tctacgaccacgtactcatgcgccctattcagactcgctttcgctgcggc

SEQ ID NO: 324 - DNA, 50 bp, synthetic construct
  misc_feature 1..50: note = DNA Probe
  source 1..50: mol_type = other DNA
taaccttgcacgggatcgtaactcgccggttcattctacaaaaggcacgc

SEQ ID NO: 325 - DNA, 50 bp, synthetic construct
  misc_feature 1..50: note = DNA Probe
  source 1..50: mol_type = other DNA
ggctctgactacttgtaggcacacggtttcaggatctctttcactcccct

SEQ ID NO: 326 - DNA, 50 bp, synthetic construct
  misc_feature 1..50: note = DNA Probe
  source 1..50: mol_type = other DNA
acctttccctcacggtactggttcactatcggtcactagggagtatttag

SEQ ID NO: 327 - DNA, 50 bp, synthetic construct
  misc_feature 1..50: note = DNA Probe
  source 1..50: mol_type = other DNA
ctcccggattccgacggaatttcacgtgttccgccgtactcaggatccac

SEQ ID NO: 328 - DNA, 50 bp, synthetic construct
  misc_feature 1..50: note = DNA Probe
  source 1..50: mol_type = other DNA
gttttgactacagggctgttacctcctatggcgggcctttccagacctct

SEQ ID NO: 329 - DNA, 50 bp, synthetic construct
  misc_feature 1..50: note = DNA Probe
  source 1..50: mol_type = other DNA
ctttgtaactccgtacagagtgtcctacaaccccaagaggcaagcctctt

SEQ ID NO: 330 - DNA, 50 bp, synthetic construct
  misc_feature 1..50: note = DNA Probe
  source 1..50: mol_type = other DNA
cgtttcgctcgccgctactcagggaatcgcatttgctttctcttcctccg

SEQ ID NO: 331 - DNA, 50 bp, synthetic construct
  misc_feature 1..50: note = DNA Probe
  source 1..50: mol_type = other DNA
cagttccccgggtctgccttctcatatcctatgaattcagatatggatac

SEQ ID NO: 332 - DNA, 50 bp, synthetic construct
  misc_feature 1..50: note = DNA Probe
  source 1..50: mol_type = other DNA
ggtgggtttccccattcggaaatctccggatcaaagcttgcttacagctc

SEQ ID NO: 333 - DNA, 50 bp, synthetic construct
  misc_feature 1..50: note = DNA Probe
  source 1..50: mol_type = other DNA
tgttcgtcccgtccttcatcggctcctagtgccaaggcatccaccgtgcg

SEQ ID NO: 334 - DNA, 50 bp, synthetic construct
  misc_feature 1..50: note = DNA Probe
  source 1..50: mol_type = other DNA
aaactagattcgaatataacaaaacattacatcctcatccaatccctttt

SEQ ID NO: 335 - DNA, 50 bp, synthetic construct
  misc_feature 1..50: note = DNA Probe
  source 1..50: mol_type = other DNA
gcggtgtgtgcaaggagcagggacgtattcaccgcgcgattgtgacacgc

SEQ ID NO: 336 - DNA, 50 bp, synthetic construct
  misc_feature 1..50: note = DNA Probe
  source 1..50: mol_type = other DNA
gcctttcggcgtcggaacccattgtctcagccattgtagcccgcgtgttg

SEQ ID NO: 337 - DNA, 50 bp, synthetic construct
  misc_feature 1..50: note = DNA Probe
  source 1..50: mol_type = other DNA
gcatacggacctaccgtcgtccactccttcctcctatttatcataggcgg

SEQ ID NO: 338 - DNA, 50 bp, synthetic construct
  misc_feature 1..50: note = DNA Probe
  source 1..50: mol_type = other DNA
cggcatccaaaaaaggatccgctggtaactaagagcgtgggtctcgctcg

SEQ ID NO: 339 - DNA, 50 bp, synthetic construct
  misc_feature 1..50: note = DNA Probe
  source 1..50: mol_type = other DNA
caacctggctatcatacagctgtcgcctctggtgagatgtccggcgttga

SEQ ID NO: 340 - DNA, 50 bp, synthetic construct
  misc_feature 1..50: note = DNA Probe
  source 1..50: mol_type = other DNA
aggctccacgcgttgtggtgctcccccgccaattcctttaagtttcagtc

SEQ ID NO: 341 - DNA, 50 bp, synthetic construct
  misc_feature 1..50: note = DNA Probe
  source 1..50: mol_type = other DNA
ccaggcggcggacttaacagcttcccttcggcactgggacagctcaaagc

SEQ ID NO: 342 - DNA, 50 bp, synthetic construct
  misc_feature 1..50: note = DNA Probe
  source 1..50: mol_type = other DNA
tccgcatcgtttacagctaggactacccgggtatctaatccggttcgcgc

SEQ ID NO: 343 - DNA, 50 bp, synthetic construct
  misc_feature 1..50: note = DNA Probe
  source 1..50: mol_type = other DNA
ttcccacagttaagctgcaggatttcaccagagacttattaaaccggcta

SEQ ID NO: 344 - DNA, 50 bp, synthetic construct
  misc_feature 1..50: note = DNA Probe
  source 1..50: mol_type = other DNA
ctcttattccaaaagctctttacactaatgaaaagccatcccgttaagaa

SEQ ID NO: 345 - DNA, 50 bp, synthetic construct
  misc_feature 1..50: note = DNA Probe
  source 1..50: mol_type = other DNA
cccccgtcgcgatttctcacattgcggaggtttcgcgcctgctgcacccc

SEQ ID NO: 346 - DNA, 50 bp, synthetic construct
  misc_feature 1..50: note = DNA Probe
  source 1..50: mol_type = other DNA
ttgtctcaggttccatctccgggctcttgctctcacaacccgtaccgatc

SEQ ID NO: 347 - DNA, 50 bp, synthetic construct
  misc_feature 1..50: note = DNA Probe
  source 1..50: mol_type = other DNA
cattacctaaccaactacctaatcggccgcagacccatccttaggcgaaa

SEQ ID NO: 348 - DNA, 50 bp, synthetic construct
  misc_feature 1..50: note = DNA Probe
  source 1..50: mol_type = other DNA
aaaccattacaggaataattgcctatccagtattatccccagtttcccag

SEQ ID NO: 349 - DNA, 50 bp, synthetic construct
  misc_feature 1..50: note = DNA Probe
  source 1..50: mol_type = other DNA
aagggtaggttatccacgtgttactgagccgtacgccacgagcctaaact

SEQ ID NO: 350 - DNA, 50 bp, synthetic construct
  misc_feature 1..50: note = DNA Probe
  source 1..50: mol_type = other DNA
acctagcgcgtagctgcccggcactgccttatcagacaaccggtcgacca

SEQ ID NO: 351 - DNA, 50 bp, synthetic construct
  misc_feature 1..50: note = DNA Probe
  source 1..50: mol_type = other DNA
cgttcctctcgtactggagccaccttcccctcagactactaacacatcca

SEQ ID NO: 352 - DNA, 50 bp, synthetic construct
  misc_feature 1..50: note = DNA Probe
  source 1..50: mol_type = other DNA
cctgtctcacgacggtctaaacccagctcacgttcccctttaatgggcga

SEQ ID NO: 353 - DNA, 50 bp, synthetic construct
  misc_feature 1..50: note = DNA Probe
  source 1..50: mol_type = other DNA
ggtgctgctgcacacccaggatggaaagaaccgacatcgaagtagcaagc

SEQ ID NO: 354 - DNA, 50 bp, synthetic construct
  misc_feature 1..50: note = DNA Probe
  source 1..50: mol_type = other DNA
ggctcttgcctgcgaccacccagttatccccgaggtagtttttctgtcat

SEQ ID NO: 355 - DNA, 50 bp, synthetic construct
  misc_feature 1..50: note = DNA Probe
  source 1..50: mol_type = other DNA
aggaggactctgaggttcgctaggcccggctttcgcctctggatttcttg

SEQ ID NO: 356 - DNA, 50 bp, synthetic construct
  misc_feature 1..50: note = DNA Probe
  source 1..50: mol_type = other DNA
caaagtaagttagaaacacagtcataagaaagtggtgtctcaagaacgaa

SEQ ID NO: 357 - DNA, 50 bp, synthetic construct
  misc_feature 1..50: note = DNA Probe
  source 1..50: mol_type = other DNA
gacttataatcgaattctcccacttacactgcatacctataaccaagctt

SEQ ID NO: 358 - DNA, 50 bp, synthetic construct
  misc_feature 1..50: note = DNA Probe
  source 1..50: mol_type = other DNA
gtaaaactctacggggtcttcgcttcccaatggaagactctggcttgtgc

SEQ ID NO: 359 - DNA, 50 bp, synthetic construct
  misc_feature 1..50: note = DNA Probe
  source 1..50: mol_type = other DNA
tcactaagttctagctagggacagtggggacctcgttctaccattcatgc

SEQ ID NO: 360 - DNA, 50 bp, synthetic construct
  misc_feature 1..50: note = DNA Probe
  source 1..50: mol_type = other DNA
cgacaaggcatttcgctaccttaagagggttatagttacccccgccgttt

SEQ ID NO: 361 - DNA, 50 bp, synthetic construct
  misc_feature 1..50: note = DNA Probe
  source 1..50: mol_type = other DNA
aactgaactccagcttcacgtgccagcactgggcaggtgtcgccctctgt

SEQ ID NO: 362 - DNA, 50 bp, synthetic construct
  misc_feature 1..50: note = DNA Probe
  source 1..50: mol_type = other DNA
ctagcagagagctatgtttttattaaacagtcgggcccccctagtcactg

SEQ ID NO: 363 - DNA, 50 bp, synthetic construct
  misc_feature 1..50: note = DNA Probe
  source 1..50: mol_type = other DNA
ttaaaacgccttagcctactcagctaggggcacctgtgacggatctcggt

SEQ ID NO: 364 - DNA, 50 bp, synthetic construct
  misc_feature 1..50: note = DNA Probe
  source 1..50: mol_type = other DNA
acaaaactaactcccttttcaaggactccatgaatcagttaaaccagtac

SEQ ID NO: 365 - DNA, 50 bp, synthetic construct
  misc_feature 1..50: note = DNA Probe
  source 1..50: mol_type = other DNA
ataatgcctacacctggttctcgctattacacctctccccaggcttaaac

SEQ ID NO: 366 - DNA, 50 bp, synthetic construct
  misc_feature 1..50: note = DNA Probe
  source 1..50: mol_type = other DNA
caatcctacaaaacatatctcgaagtgtcagaaattagccctcaacgtca

SEQ ID NO: 367 - DNA, 50 bp, synthetic construct
  misc_feature 1..50: note = DNA Probe
  source 1..50: mol_type = other DNA
ctttgctgctactactaccaggatccacatacctgcaaggtccaaaggaa

SEQ ID NO: 368 - DNA, 50 bp, synthetic construct
  misc_feature 1..50: note = DNA Probe
  source 1..50: mol_type = other DNA
caacccacacaggtcgccactctacacaatcaccaaaaaaaaggtgttcc

SEQ ID NO: 369 - DNA, 50 bp, synthetic construct
  misc_feature 1..50: note = DNA Probe
  source 1..50: mol_type = other DNA
ggattaattcccgtccattttaggtgcctctgacctcgatgggtgatctg

SEQ ID NO: 370 - DNA, 50 bp, synthetic construct
  misc_feature 1..50: note = DNA Probe
  source 1..50: mol_type = other DNA
agggtggctgcttctaagcccaccttcccattgtcttgggccaaagactc

SEQ ID NO: 371 - DNA, 50 bp, synthetic construct
  misc_feature 1..50: note = DNA Probe
  source 1..50: mol_type = other DNA
gtatttaggggccttaaccatagtctgagttgtttctctttcgggacaca

SEQ ID NO: 372 - DNA, 50 bp, synthetic construct
  misc_feature 1..50: note = DNA Probe
  source 1..50: mol_type = other DNA
cctcactccaaccttctacgacggtgacgagttcggagttttacagtacg

SEQ ID NO: 373 - DNA, 50 bp, synthetic construct
  misc_feature 1..50: note = DNA Probe
  source 1..50: mol_type = other DNA
ccctaaacgtccaattagtgctctaccccgccaccaacctccagtcaggc

SEQ ID NO: 374 - DNA, 50 bp, synthetic construct
  misc_feature 1..50: note = DNA Probe
  source 1..50: mol_type = other DNA
aatagatcgaccggcttcgggtttcaatgctgtgattccaggccctatta

SEQ ID NO: 375 - DNA, 50 bp, synthetic construct
  misc_feature 1..50: note = DNA Probe
  source 1..50: mol_type = other DNA
acaacgctgcgggcatatcggtttccctacgactacaaggataaaaacct

SEQ ID NO: 376 - DNA, 50 bp, synthetic construct
  misc_feature 1..50: note = DNA Probe
  source 1..50: mol_type = other DNA
acaaagaactccctggcccgtgtttcaagacggacgatgcaacactagtc

SEQ ID NO: 377 - DNA, 50 bp, synthetic construct
  misc_feature 1..50: note = DNA Probe
  source 1..50: mol_type = other DNA
acaatgttaccactgattctttcggaagaattcattccttacgcgccaca

SEQ ID NO: 378 - DNA, 50 bp, synthetic construct
  misc_feature 1..50: note = DNA Probe
  source 1..50: mol_type = other DNA
ctggtttcaggtacttttcacccccctataggggtacttttcagcattcc

SEQ ID NO: 379 - DNA, 50 bp, synthetic construct
  misc_feature 1..50: note = DNA Probe
  source 1..50: mol_type = other DNA
ctctatcggtcttgagacgtatttagaattggaagttgatgcctcccaca

SEQ ID NO: 380 - DNA, 50 bp, synthetic construct
  misc_feature 1..50: note = DNA Probe
  source 1..50: mol_type = other DNA
atcaccctctacggttctaaaattccaaataaaattcgatttatcccacg

SEQ ID NO: 381 - DNA, 50 bp, synthetic construct
  misc_feature 1..50: note = DNA Probe
  source 1..50: mol_type = other DNA
tctatacaccacatctccctaatattactaaaagggattcagtttgttct

SEQ ID NO: 382 - DNA, 50 bp, synthetic construct
  misc_feature 1..50: note = DNA Probe
  source 1..50: mol_type = other DNA
gccgttactaacgacatcgcatattgctttcttttcctccgcctactaag

SEQ ID NO: 383 - DNA, 50 bp, synthetic construct
  misc_feature 1..50: note = DNA Probe
  source 1..50: mol_type = other DNA
gggttcccaatcctacacggatcaacacaaaaaaaatgtgctaggaagtc

SEQ ID NO: 384 - DNA, 50 bp, synthetic construct
  misc_feature 1..50: note = DNA Probe
  source 1..50: mol_type = other DNA
actactgggatcgaaacgagaccaggtataacccccatgctatgaccgca

SEQ ID NO: 385 - protein, 50 aa, Mus musculus
GCGTATGCCTGGAGAATTGGAATTCTTGTTACTCATACTAACAGTGTTGC

SEQ ID NO: 386 - protein, 50 aa, Mus musculus
GATTAACCCAATTTTAAGTTTAGGAAGTTGGTGTAAATTATGGAATTAAT

SEQ ID NO: 387 - protein, 50 aa, Mus musculus
AGCTTGAACGCTTTCTTTATTGGTGGCTGCTTTTAGGCCTACAATGGTTA

SEQ ID NO: 388 - protein, 50 aa, Mus musculus
ATTATTCACTATTAAAGGTTTTTTCCGTTCCAGAAGAGCTGTCCCTCTTT

SEQ ID NO: 389 - protein, 50 aa, Mus musculus
CTTACTTTTTGATTTTGTTGTTTTTTTAGCAAGTTTAAAATTGAACTTAA

SEQ ID NO: 390 - protein, 50 aa, Mus musculus
AACCAGCTATCACCAAGCTCGTTAGGCTTTTCACCTCTACCTAAAAATCT

SEQ ID NO: 391 - protein, 50 aa, Mus musculus
AATACTTGTAATGCTAGAGGTGATGTTTTTGGTAAACAGGCGGGGTTCTT

SEQ ID NO: 392 - protein, 50 aa, Mus musculus
TTTATCTTTTTGGATCTTTCCTTTAGGCATTCCGGTGTTGGGTTAACAGA

SEQ ID NO: 393 - protein, 50 aa, Mus musculus
TTATTTATAGTGTGATTATTGCCTATAGTCTGATTAACTAACAATGGTTA

SEQ ID NO: 394 - protein, 50 aa, Rattus norvegicus
AGTGATTGTAGTTGTTTATTCACTATTTAAGGTTTTTTCCTTTTCCTAAA

SEQ ID NO: 395 - protein, 50 aa, Rattus norvegicus
TGGCTATATTTTAAGTTTACATTTTGATTTGTTGTTCTGATGGTAAGCTT

SEQ ID NO: 396 - protein, 50 aa, Rattus norvegicus
TTTTTTTAATCTTTCCTTAAAGCACGCCTGTGTTGGGCTAACGAGTTAGG

SEQ ID NO: 397 - protein, 50 aa, Rattus norvegicus
TGTTGGGTTAGTACCTATGATTCGATAATTGACAATGGTTATCCGGGTTG

SEQ ID NO: 398 - protein, 50 aa, Rattus norvegicus
AGGAGAATTGGTTCTTGTTACTCATATTAACAGTATTTCATCTATGGATC

SEQ ID NO: 399 - protein, 50 aa, Rattus norvegicus
TTTGTGATATAGGAATTTATTGAGGTTTGTGGAATTAGTGTGTGTAAGTA

SEQ ID NO: 400 - protein, 50 aa, Mus musculus
GCCGGGGAGTGGGTCTTCCGTACGCCACATTTCCCACGCCGCGACGCGCG

SEQ ID NO: 401 - protein, 50 aa, Mus musculus
ACCTCGGGCCCCCGGGCGGGGCCCTTCACCTTCATTGCGCCACGGCGGCT

SEQ ID NO: 402 - protein, 50 aa, Mus musculus
TCGCGTCCAGAGTCGCCGCCGCCGCCGGCCCCCCGAGTGTCCGGGCCCCC

SEQ ID NO: 403 - protein, 50 aa, Mus musculus
CGCTGGTTCCTCCCGCTCCGGAACCCCCGCGGGGTTGGACCCGCCGCCCC

SEQ ID NO: 404 - protein, 50 aa, Mus musculus
CGCCGACCCCCGACCCGCCCCCCGACGGGAAGAAGGAGGGGGGAAGAGAG

SEQ ID NO: 405 - protein, 50 aa, Mus musculus
GGGACGACGGGGCCCCGCGGGGAAGAGGGGAGGGCGGGCCCGGGCGGAAA

SEQ ID NO: 406 - protein, 50 aa, Mus musculus
GGCGCCGCGCGGAAAACCGCGGCCCGGGGGGCGGACCCGGCGGGGGAACA

SEQ ID NO: 407 - protein, 50 aa, Mus musculus
CCCCCACACGCGCGGGACACGCCCGCCCGCCCCCGCCACGCACCTCGGGA

SEQ ID NO: 408 - protein, 50 aa, Mus musculus
CACCCGCTTTGGGCTGCATTCCCAAGCAACCCGACTCCGGGAAGACCCGA

SEQ ID NO: 409 - protein, 50 aa, Mus musculus
TGGAGCGAGGCCCCGCGGGGAGGGGACCCGCGCCGGCACCCGCCGGGCTC

SEQ ID NO: 410 - protein, 50 aa, Mus musculus
CGAGGCCGGCGTGCCCCGACCCCGACGCGAGGACGGGGCCGGGCGCCGGG

SEQ ID NO: 411 - protein, 50 aa, Mus musculus
TCCCCGGAGCGGGTCGCGCCCGCCCGCACGCGCGGGACGGACGCTTGGCG

SEQ ID NO: 412 - protein, 50 aa, Mus musculus
TCCACACGAACGTGCGTTCAACGTGACGGGCGAGAGGGCGGCCCCCTTTC

SEQ ID NO: 413 - protein, 50 aa, Mus musculus
TCCCAAGACGAACGGCTCTCCGCACCGGACCCCGGTCCCGACGCCCGGCG

SEQ ID NO: 414 - protein, 50 aa, Mus musculus
CCGCCGCGGGGACGACGCGGGGACCCCGCCGAGCGGGGACGGACGGGGAC

SEQ ID NO: 415 - protein, 50 aa, Mus musculus
GCACCGCCACGGTGGAAGTGCGCCCGGCGGCGGCCGGTCGCCGGCCGGGG

SEQ ID NO: 416 - protein, 50 aa, Mus musculus
CCCACCGGGCCCCGAGAGAGGCGACGGAGGGGGGTGGGAGAGCGGTCGCG

SEQ ID NO: 417 - protein, 50 aa, Mus musculus
CCCGGCCCCCACCCCCACGCCCGCCCGGGAGGCGGACGGGGGGAGAGGGA

SEQ ID NO: 418 - protein, 50 aa, Mus musculus
TATCTGGCTTCCTCGGCCCCGGGATTCGGCGAAAGCGCGGCCGGAGGGCT

SEQ ID NO: 419 - protein, 50 aa, Mus musculus
CGCCGCCGACCCCGTGCGCTCGGCTTCGTCGGGAGACGCGTGACCGACGG

SEQ ID NO: 420 - protein, 50 aa, Mus musculus
GCGCCCCCCCGCACCCGCCCCGTCCCCCCCGCGGACGGGGAAGAAGGGAG

SEQ ID NO: 421 - protein, 50 aa, Mus musculus
CGAACCCCGGGAACCCCCGACCCCGCGGAGGGGGAAGGGGGAGGACGAGG

SEQ ID NO: 422 - protein, 50 aa, Mus musculus
CACCCGGGGGGGCGACGAGGCGGGGACCCGCCGGACGGGGACGGACGGGG

SEQ ID NO: 423 - protein, 50 aa, Mus musculus
GCCAACCGAGGCTCCTTCGGCGCTGCCGTATCGTTCCGCTTGGGCGGATT

SEQ ID NO: 424 - protein, 50 aa, Mus musculus
CCCGGGCCCCCGGACCCCCGAGAGGGACGACGGAGGCGACGGGGGGTGGG

SEQ ID NO: 425 - protein, 50 aa, Mus musculus
TGGGAGGGGCGGCCCGGCCCCCGCGACCGCCCCCCTTTCCGCCACCCCAC

SEQ ID NO: 426 - protein, 50 aa, Mus musculus
GGGAGAGGCCGGGGGGAGAGCGCGGCGACGGGTATCCGGCTCCCTCGGCC

SEQ ID NO: 427 - protein, 50 aa, Mus musculus
CGCTGCTGCCGGGGGGCTGTAACACTCGGGGCGGGGTGGTCCGGCGCCCA

SEQ ID NO: 428 - protein, 50 aa, Mus musculus
CGCCGCCGACCCCGTGCGCTCGGCTTCGCTCCCCCCCACCCCGAGAAGGG